SEQUENCE LISTING (80 sequences)

SEQ ID NO: 1 - RNA, 22 nt, Artificial Sequence
  synthetic
gucauacacggcucuccucucu

SEQ ID NO: 2 - RNA, 7 nt, Artificial Sequence
  synthetic
uguauga

SEQ ID NO: 3 - RNA, 8 nt, Artificial Sequence
  synthetic
guguauga

SEQ ID NO: 4 - RNA, 9 nt, Artificial Sequence
  synthetic
cguguauga

SEQ ID NO: 5 - RNA, 10 nt, Artificial Sequence
  synthetic
ccguguauga

SEQ ID NO: 6 - RNA, 11 nt, Artificial Sequence
  synthetic
gccguguauga

SEQ ID NO: 7 - RNA, 12 nt, Artificial Sequence
  synthetic
agccguguauga

SEQ ID NO: 8 - RNA, 13 nt, Artificial Sequence
  synthetic
gagccguguauga

SEQ ID NO: 9 - DNA, 14 bp, Artificial Sequence
  synthetic
agagccguguauga

SEQ ID NO: 10 - DNA, 15 bp, Artificial Sequence
  synthetic
gagagccguguauga

SEQ ID NO: 11 - DNA, 16 bp, Artificial Sequence
  synthetic
ggagagccguguauga

SEQ ID NO: 12 - DNA, 17 bp, Artificial Sequence
  synthetic
aggagagccguguauga

SEQ ID NO: 13 - DNA, 18 bp, Artificial Sequence
  synthetic
gaggagagccguguauga

SEQ ID NO: 14 - DNA, 19 bp, Artificial Sequence
  synthetic
agaggagagccguguauga

SEQ ID NO: 15 - DNA, 20 bp, Artificial Sequence
  synthetic
gagaggagagccguguauga

SEQ ID NO: 16 - DNA, 8 bp, Artificial Sequence
  synthetic
uguaugac

SEQ ID NO: 17 - DNA, 9 bp, Artificial Sequence
  synthetic
guguaugac

SEQ ID NO: 18 - DNA, 10 bp, Artificial Sequence
  synthetic
cguguaugac

SEQ ID NO: 19 - DNA, 11 bp, Artificial Sequence
  synthetic
ccguguaugac

SEQ ID NO: 20 - DNA, 12 bp, Artificial Sequence
  synthetic
gccguguaugac

SEQ ID NO: 21 - DNA, 13 bp, Artificial Sequence
  synthetic
agccguguaugac

SEQ ID NO: 22 - DNA, 14 bp, Artificial Sequence
  synthetic
gagccguguaugac

SEQ ID NO: 23 - DNA, 15 bp, Artificial Sequence
  synthetic
agagccguguaugac

SEQ ID NO: 24 - DNA, 16 bp, Artificial Sequence
  synthetic
gagagccguguaugac

SEQ ID NO: 25 - DNA, 17 bp, Artificial Sequence
  synthetic
ggagagccguguaugac

SEQ ID NO: 26 - DNA, 18 bp, Artificial Sequence
  synthetic
aggagagccguguaugac

SEQ ID NO: 27 - DNA, 19 bp, Artificial Sequence
  synthetic
gaggagagccguguaugac

SEQ ID NO: 28 - DNA, 20 bp, Artificial Sequence
  synthetic
agaggagagccguguaugac

SEQ ID NO: 29 - DNA, 21 bp, Artificial Sequence
  synthetic
gagaggagagccguguaugac

SEQ ID NO: 30 - DNA, 7 bp, Artificial Sequence
  synthetic
tgtatga

SEQ ID NO: 31 - protein, 440 aa, Artificial Sequence
  synthetic
MGSSHLLNKGLPLGVRPPIMNGPLHPRPLVALLDGRDCTVEMPILKDVATVAFCDAQSTQEIHEKVLNEAVGALMYHTITLTREDLEKFKALRIIVRIGSGFDNIDIKSAGDLGIAVCNVPAASVEETADSTLCHILNLYRRATWLHQALREGTRVQSVEQIREVASGAARIRGETLGIIGLGRVGQAVALRAKAFGFNVLFYDPYLSDGVERALGLQRVSTLQDLLFHSDCVTLHCGLNEHNHHLINDFTVKQMRQGAFLVNTARGGLVDEKALAQALKEGRIRGAALDVHESEPFSFSQGPLKDAPNLICTPHAAWYSEQASIEMREEAAREIRRAITGRIPDSLKNCVNKDHLTAATHWASMDPAVVHPELNGAAYRYPPGVVGVAPTGIPAAVEGIVPSAMSLSHGLPPVAHPPHAPSPGQTVKPEADRDHASDQL

SEQ ID NO: 32 - protein, 429 aa, Artificial Sequence
  synthetic
MSGVRPPIMNGPLHPRPLVALLDGRDCTVEMPILKDVATVAFCDAQSTQEIHEKVLNEAVGALMYHTITLTREDLEKFKALRIIVRIGSGFDNIDIKSAGDLGIAVCNVPAASVEETADSTLCHILNLYRRATWLHQALREGTRVQSVEQIREVASGAARIRGETLGIIGLGRVGQAVALRAKAFGFNVLFYDPYLSDGVERALGLQRVSTLQDLLFHSDCVTLHCGLNEHNHHLINDFTVKQMRQGAFLVNTARGGLVDEKALAQALKEGRIRGAALDVHESEPFSFSQGPLKDAPNLICTPHAAWYSEQASIEMREEAAREIRRAITGRIPDSLKNCVNKDHLTAATHWASMDPAVVHPELNGAAYRYPPGVVGVAPTGIPAAVEGIVPSAMSLSHGLPPVAHPPHAPSPGQTVKPEADRDHASDQL

SEQ ID NO: 33 - DNA, 22 bp, Artificial Sequence
  synthetic
agaggcuggccgugaugaauuc

SEQ ID NO: 34 - DNA, 22 bp, Artificial Sequence
  synthetic
agucauacacggcucuccucuc

SEQ ID NO: 35 - DNA, 13 bp, Artificial Sequence
  synthetic
gagccgtgtatga

SEQ ID NO: 36 - protein, 472 aa, Artificial Sequence
  synthetic
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRNLSYWESHCDMINGTDAASFPPFVEKSQVLQFFSSDICRSIYAVFESDVNLKGIPVYRFVLPSKAFASPVENPDNYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK

SEQ ID NO: 37 - protein, 288 aa, Artificial Sequence
  synthetic
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRNLSYWESHCDMINGTDAASFPPFVEKSQVLQFFSSDICRETCVHFTSSFSVCKS

SEQ ID NO: 38 - protein, 433 aa, Artificial Sequence
  synthetic
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRSIYAVFESDVNLKGIPVYRFVLPSKAFASPVENPDNYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK

SEQ ID NO: 39 - protein, 412 aa, Artificial Sequence
  synthetic
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAYNNTADGVYKVFNGKDNISKVAIIDTYKGKRNLSYWESHCDMINGTDAASFPPFVEKSQVLQFFSSDICRSIYAVFESDVNLKGIPVYRFVLPSKAFASPVENPDNYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK

SEQ ID NO: 40 - protein, 798 aa, Artificial Sequence
  synthetic
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 41 - protein, 271 aa, Artificial Sequence
  synthetic
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNLFL

SEQ ID NO: 42 - protein, 803 aa, Artificial Sequence
  synthetic
MDETSPRLEEDWKKVLQREAGWQCAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 43 - protein, 786 aa, Artificial Sequence
  synthetic
MDEGYFCAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 44 - protein, 289 aa, Artificial Sequence
  synthetic
MDEGYFCAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGVKTNLISK

SEQ ID NO: 45 - protein, 276 aa, Artificial Sequence
  synthetic
MDETSPRLEEDWKKVLQREAGWQCAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNLFL

SEQ ID NO: 46 - protein, 138 aa, Artificial Sequence
  synthetic
MDEGYFCAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLVRTLPTV

SEQ ID NO: 47 - protein, 301 aa, Artificial Sequence
  synthetic
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGVKTNLISK

SEQ ID NO: 48 - protein, 671 aa, Artificial Sequence
  synthetic
MPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 49 - DNA, 7 bp, Artificial Sequence
  synthetic
ucauaca

SEQ ID NO: 50 - DNA, 7 bp, Artificial Sequence
  synthetic
gaggcug

SEQ ID NO: 51 - DNA, 8 bp, Artificial Sequence
  synthetic
gtgtatga

SEQ ID NO: 52 - DNA, 9 bp, Artificial Sequence
  synthetic
cgtgtatga

SEQ ID NO: 53 - DNA, 10 bp, Artificial Sequence
  synthetic
ccgtgtatga

SEQ ID NO: 54 - DNA, 11 bp, Artificial Sequence
  synthetic
gccgtgtatga

SEQ ID NO: 55 - DNA, 12 bp, Artificial Sequence
  synthetic
agccgtgtatga

SEQ ID NO: 56 - DNA, 14 bp, Artificial Sequence
  synthetic
agagccgtgtatga

SEQ ID NO: 57 - DNA, 15 bp, Artificial Sequence
  synthetic
gagagccgtgtatga

SEQ ID NO: 58 - DNA, 16 bp, Artificial Sequence
  synthetic
ggagagccgtgtatga

SEQ ID NO: 59 - DNA, 17 bp, Artificial Sequence
  synthetic
aggagagccgtgtatga

SEQ ID NO: 60 - DNA, 18 bp, Artificial Sequence
  synthetic
gaggagagccgtgtatga

SEQ ID NO: 61 - DNA, 20 bp, Artificial Sequence
  synthetic
gagaggagagccgtgtatga

SEQ ID NO: 62 - DNA, 20 bp, Artificial Sequence
  synthetic
gagaggagagccgtgtatga

SEQ ID NO: 63 - DNA, 8 bp, Artificial Sequence
  synthetic
tgtatgac

SEQ ID NO: 64 - DNA, 9 bp, Artificial Sequence
  synthetic
gtgtatgac

SEQ ID NO: 65 - DNA, 10 bp, Artificial Sequence
  synthetic
cgtgtatgac

SEQ ID NO: 66 - DNA, 11 bp, Artificial Sequence
  synthetic
ccgtgtatgac

SEQ ID NO: 67 - DNA, 12 bp, Artificial Sequence
  synthetic
gccgtgtatgac

SEQ ID NO: 68 - DNA, 13 bp, Artificial Sequence
  synthetic
agccgtgtatgac

SEQ ID NO: 69 - DNA, 14 bp, Artificial Sequence
  synthetic
gagccgtgtatgac

SEQ ID NO: 70 - DNA, 15 bp, Artificial Sequence
  synthetic
agagccgtgtatgac

SEQ ID NO: 71 - DNA, 16 bp, Artificial Sequence
  synthetic
gagagccgtgtatgac

SEQ ID NO: 72 - DNA, 17 bp, Artificial Sequence
  synthetic
ggagagccgtgtatgac

SEQ ID NO: 73 - DNA, 18 bp, Artificial Sequence
  synthetic
aggagagccgtgtatgac

SEQ ID NO: 74 - DNA, 19 bp, Artificial Sequence
  synthetic
gaggagagccgtgtatgac

SEQ ID NO: 75 - DNA, 20 bp, Artificial Sequence
  synthetic
agaggagagccgtgtatgac

SEQ ID NO: 76 - DNA, 21 bp, Artificial Sequence
  synthetic
gagaggagagccgtgtatgac

SEQ ID NO: 77 - DNA, 20 bp, Artificial Sequence
  synthetic
agaggagagccgtgtatgac

SEQ ID NO: 78 - protein, 476 aa, Artificial Sequence
  synthetic
MSGPTWLPPKQPEPARAPQGRAIPRGTPGPPPAHGAALQPHPRVNFCPLPSEQCYQAPGGPEDRGPAWVGSHGVLQHTQGLPADRGGLRPGSLDAEIDLLSSTLAELNGGRGHASRRPDRQAYEPPPPPAYRTGSLKPNPASPLPASPYGGPTPASYTTASTPAGPAFPVQVKVAQPVRGCGPPRRGASQASGPLPGPHFPLPGRGEVWGPGYRSQREPGPGAKEEAAGVSGPAGRGRGGEHGPQVPLSQPPEDELDRLTKKLVHDMNHPPSGEYFGQCGGCGEDVVGDGAGVVALDRVFHVGCFVCSTCRAQLRGQHFYAVERRAYCEGCYVATLEKCATCSQPILDRILRAMGKAYHPGCFTCVVCHRGLDGIPFTVDATSQIHCIEDFHRKFAPRCSVCGGAIMPEPGQEETVRIVALDRSFHIGCYKCEECGLLLSSEGECQGCYPLDGHILCKACSAWRIQELSATVTTDC

SEQ ID NO: 79 - protein, 105 aa, Artificial Sequence
  synthetic
SGPTWLPPKQPEPARAPQGRAIPRGTPGPPPAHGAVLPGPRGTGGSGAGVGGVPWSTPAHAGAPCRQGGPSPWKPGRRDRLAEQHAGRAEWGSGSCVTATRPTGI

SEQ ID NO: 80 - protein, 80 aa, Artificial Sequence
  synthetic
MSGPTWLPPKQPEPARAPQGRAIPRGTPGPPPAHGAGAPCRQGGPSPWKPGRRDRLAEQHAGRAEWGSGSCVTATRPTGI

(19) United States
(12) Patent Application Publication
(10) Pub. No.: US 2024/0050461 A1
(43) Pub. Date: Feb. 15, 2024

(54) USE OF MIRNA-485 INHIBITORS FOR INDUCING HAIR GROWTH

(71) Applicant: BIORCHESTRA CO., LTD., Daejeon (KR)

(72) Inventors: Jin-Hyeob RYU, Daejeon (KR); Begum SHAHNAZ, Daejeon (KR); Jamil MD HOSSAIN, Daejeon (KR); Hyun Su MIN, Daejeon (KR); Yu Na LIM, Daejeon (KR)

(73) Assignee: BIORCHESTRA CO., LTD., Daejeon (KR)

(21) Appl. No.: 18/259,007

(22) PCT Filed: Dec. 23, 2021
(86) PCT No.: PCT/IB2021/062226

§ 371 Date: Jun. 22, 2023

Related U.S. Application Data
(60) Provisional application No. 63/130,121, filed on Dec. 23, 2020.

(51) Int. Cl. A61K 31/7125; A61K 47/18; A61K 47/22; A61K 47/34; A61K 47/69; A61P 17/14

(52) U.S. Cl. CPC A61K 31/7125; A61K 47/183; A61K 47/22; A61K 47/34; A61K 47/6907; A61P 17/14

(57) ABSTRACT

The present disclosure includes the use of a miRNA inhibitor for inducing hair growth, increasing the hair density, increasing the follicular density, increasing the hair shaft thickness, increasing hair length, preventing hair loss, reducing hair loss, or any combination thereof in a subject in need thereof. In some aspects, the subject has one or more disorders selected from the group consisting of alopecia greata, androgenic alopecia, alopecia areata, alopecia universalis, involutional alopecia, trichotillomania, telogen effluvium, anagen effluvium, cicatricial, alopecia, scarring alopecia, scalp thinning, hair shaft abnormalities, infectious hair disorders, genetic disorders, and hair loss due to chemotherapy, hormonal imbalance, fungal infection, medication intake, chemical hair treatment, or aging.

### CROSS REFERENCE TO RELATED APPLICATIONS

This application claims the benefit of the filing date of U.S. Provisional Patent Application No. 63/130,121, filed Dec. 23, 2020, the contents of which are incorporated herein by reference in their entirety.

### REFERENCE TO SEQUENCE LISTING SUBMITTED ELECTRONICALLY VIA EFS-WEB

The content of the electronically submitted sequence listing in ASCII text file (Name: Sequence-Listing-4366-037PC02_SL_ST25; Size: 76,935 bytes; and Date of Creation: Dec. 21, 2021) filed with the application is herein incorporated by reference in its entirety.

### FIELD OF THE DISCLOSURE

The present disclosure provides the use of a miR-485 inhibitor (e.g., polynucleotide encoding a nucleotide molecule comprising at least one miR-485 binding site) for inducing hair growth.

### BACKGROUND OF THE DISCLOSURE

As the aged population is increasing and stress is high in modern society, there are a growing number of people under the threat of hair fall and hair loss. Although medically benign, they can cause tremendous emotional and psychosocial pressure in affected patients and their families. The cause of hair loss includes a part of the natural aging process, compromised hair growth cycle, genetic predisposition, unwanted side effects from medication and/or medical treatment, environmental causes such as allergies, infections, stress, a lack of proper diet and sleep, and/or other systematic disorders.

It is estimated that 40% of males in the United States will have some noticeable hair loss by age 35. This condition gets worse as men get older. By age 60, it is estimated that 65% of adult males in the United States will suffer from noticeable hair loss. Women also suffer from hair loss. Female hair loss, unlike in men, typically involves noticeable thinning all over the head and the hairline. The American Academy of Dermatology reports that 40% of women have noticeable hair loss by the age 40. See e.g., U.S. Pat. No. 9,173,921.

Therefore, new and more effective approaches of inducing hair growth are highly desirable.

### BRIEF SUMMARY OF THE DISCLOSURE

Provided herein is a method of inducing hair growth in a subject in need thereof comprising administering to the subject a compound that inhibits miR-485 (“miRNA inhibitor”).

Also provided herein is a method of increasing the hair density in a subject in need thereof comprising administering to the subject a compound that inhibits miR-485 (“miRNA inhibitor”).

Also provided herein is a method of increasing the follicular density in a subject in need thereof comprising administering to the subject a compound that inhibits miR-485 (“miRNA inhibitor”).

Also provided herein is a method of increasing the hair shaft thickness in a subject in need thereof comprising administering to the subject a compound that inhibits miR-485 (“miRNA inhibitor”).

Also provided herein is a method of increasing the hair length in a subject in need thereof comprising administering to the subject a compound that inhibits miR-485 (“miRNA inhibitor”).

Also provided herein is a method for preventing hair loss in a subject at risk of hair loss comprising administering to the subject a compound that inhibits miR-485 (“miRNA inhibitor”).

Also provided herein is a method for reducing hair loss in a subject in need thereof comprising administering to the subject a compound that inhibits miR-485 (miRNA inhibitor).

In some aspects, the subject has one or more disorders selected from the group consisting of alopecia greata, androgenic alopecia, alopecia areata, alopecia universalis, involutional alopecia, trichotillomania, telogen effluvium, anagen effluvium, cicatricial, alopecia, scarring alopecia, scalp thinning, hair shaft abnormalities, infectious hair disorders, genetic disorders, and hair loss due to chemotherapy, hormonal imbalance, fungal infection, medication intake, chemical hair treatment, or aging. In some aspects, the subject is a human.

In some aspects, the miRNA inhibitors described herein induce autophagy and/or treats or prevents inflammation.

In some aspects, the miRNA inhibitors described herein inhibit miR485-3p. In some aspects, the miR485-3p comprises 5′-GUCAUACACGGCUCUCCUCUCU-3′ (SEQ ID NO: 1). In some aspects, the miRNA inhibitor described herein comprises a nucleotide sequence comprising 5′-UGUAUGA-3′ (SEQ ID NO: 2) and wherein the miRNA inhibitor comprises about 6 to about 30 nucleotides in length.

In some aspects, the miRNA inhibitors described herein comprise at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides at the 5′ of the nucleotide sequence. In some aspects, the miRNA inhibitor comprises at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides at the 3′ of the nucleotide sequence.

In some aspects, the miRNA inhibitor described herein has a sequence selected from the group consisting of: 5′-UGUAUGA-3′ (SEQ ID NO: 2), 5′-GUGUAUGA-3′ (SEQ ID NO: 3), 5′-CGUGUAUGA-3′ (SEQ ID NO: 4), 5′-CCGUGUAUGA-3′ (SEQ ID NO: 5), 5′-GCCGUGUAUGA-3′ (SEQ ID NO: 6), 5′-AGCCGUGUAUGA-3′ (SEQ ID NO: 7), 5′-GAGCCGUGUAUGA-3′ (SEQ ID NO: 8), 5′-AGAGCCGUGUAUGA-3′ (SEQ ID NO: 9), 5′-GAGAGCCGUGUAUGA-3′ (SEQ ID NO: 10), 5′-GGAGAGCCGUGUAUGA-3′ (SEQ ID NO: 11), 5′-AGGAGAGCCGUGUAUGA-3′ (SEQ ID NO: 12), 5′-GAGGAGAGCCGUGUAUGA-3′ (SEQ ID NO: 13), 5′-AGAGGAGAGCCGUGUAUGA-3′ (SEQ ID NO: 14), 5′-GAGAGGAGAGCCGUGUAUGA-3′ (SEQ ID NO: 15); 5′-UGUAUGAC-3′ (SEQ ID NO: 16), 5′-GUGUAUGAC-3′ (SEQ ID NO: 17), 5′-CGUGUAUGAC-3′ (SEQ ID NO: 18), 5′-CCGUGUAUGAC-3′ (SEQ ID NO: 19), 5′-GCCGUGUAUGAC-3′ (SEQ ID NO: 20), 5′-AGCCGUGUAUGAC-3′ (SEQ ID NO: 21), 5′-GAGCCGUGUAUGAC-3′ (SEQ ID NO: 22), 5′-AGAGCCGUGUAUGAC-3′ (SEQ ID NO: 23), 5′-GAGAGCCGUGUAUGAC-3′ (SEQ ID NO: 24), 5′-GGAGAGCCGUGUAUGAC-3′ (SEQ ID NO: 25), 5′-AGGAGAGCCGUGUAUGAC-3′ (SEQ ID NO: 26), 5′-GAGGAGAGCCGUGUAUGAC-3′ (SEQ ID NO: 27), 5′-AGAGGAGAGCCGUGUAUGAC-3′ (SEQ ID NO: 28), or 5′-GAGAGGAGAGCCGUGUAUGAC-3′ (SEQ ID NO: 29).

In some aspects, the miRNA inhibitor described herein has a sequence selected from the group consisting of: 5′-TGTATGA-3′ (SEQ ID NO: 30), 5′-GTGTATGA-3′ (SEQ ID NO: 51), 5′-CGTGTATGA-3′ (SEQ ID NO: 52), 5′-CCGTGTATGA-3′ (SEQ ID NO: 53), 5′-GCCGTGTATGA-3′ (SEQ ID NO: 54), 5′-AGCCGTGTATGA-3′ (SEQ ID NO: 55), 5′-GAGCCGTGTATGA-3′ (SEQ ID NO: 35), 5′-AGAGCCGTGTATGA-3′ (SEQ ID NO: 56), 5′-GAGAGCCGTGTATGA-3′ (SEQ ID NO: 57), 5′-GGAGAGCCGTGTATGA-3′ (SEQ ID NO: 58), 5′-AGGAGAGCCGTGTATGA-3′ (SEQ ID NO: 59), 5′-GAGGAGAGCCGTGTATGA-3′ (SEQ ID NO: 60), 5′-AGAGGAGAGCCGTGTATGA-3′ (SEQ ID NO: 61), 5′-GAGAGGAGAGCCGTGTATGA-3′ (SEQ ID NO: 62); 5′-TGTATGAC-3′ (SEQ ID NO: 63), 5′-GTGTATGAC-3′ (SEQ ID NO: 64), 5′-CGTGTATGAC-3′ (SEQ ID NO: 65), 5′-CCGTGTATGAC-3′ (SEQ ID NO: 66), 5′-GCCGTGTATGAC-3′ (SEQ ID NO: 67), 5′-AGCCGTGTATGAC-3′ (SEQ ID NO: 68), 5′-GAGCCGTGTATGAC-3′ (SEQ ID NO: 69), 5′-AGAGCCGTGTATGAC-3′ (SEQ ID NO: 70), 5′-GAGAGCCGTGTATGAC-3′ (SEQ ID NO: 71), 5′-GGAGAGCCGTGTATGAC-3′ (SEQ ID NO: 72), 5′-AGGAGAGCCGTGTATGAC-3′ (SEQ ID NO: 73), 5′-GAGGAGAGCCGTGTATGAC-3′ (SEQ ID NO: 74), 5′-AGAGGAGAGCCGTGTATGAC-3′ (SEQ ID NO: 75), and 5′-GAGAGGAGAGCCGTGTATGAC-3′ (SEQ ID NO: 76).

In some aspects, the sequence of the miRNA inhibitor described herein is at least about 50%, at least about 55%, at least about 60%, at least about 65%, at least about 70%, at least about 75%, at least about 80%, at least about 85%, at least about 90%, or at least about 95% sequence identity to 5′- AGAGGAGAGCCGUGUAUGAC-3′ (SEQ ID NO: 28) or 5′-AGAGGAGAGCCGTGTATGAC-3′ (SEQ ID NO: 77). In certain aspects, the miRNA inhibitor described herein has a sequence that has at least 90% similarity to 5′-AGAGGAGAGCCGUGUAUGAC-3′ (SEQ ID NO: 28) or 5′-AGAGGAGAGCCGTGTATGAC-3′ (SEQ ID NO: 77). In some aspects, the miRNA inhibitor described herein comprises the nucleotide sequence 5′-AGAGGAGAGCCGUGUAUGAC-3′ (SEQ ID NO: 28) or 5′-AGAGGAGAGCCGTGTATGAC-3′ (SEQ ID NO: 77) with one substitution or two substitutions. In some aspects, the miRNA inhibitor described herein comprises the nucleotide sequence 5′-AGAGGAGAGCCGUGUAUGAC-3′ (SEQ ID NO: 28) or 5′-AGAGGAGAGCCGTGTATGAC-3′ (SEQ ID NO: 77). In some aspects, the miRNA inhibitor described herein comprises the nucleotide sequence 5′-AGAGGAGAGCCGUGUAUGAC-3′ (SEQ ID NO: 28).

In some aspects, the miRNA inhibitors described herein comprise at least one modified nucleotide. In certain aspects, the at least one modified nucleotide is a locked nucleic acid (LNA), an unlocked nucleic acid (UNA), an arabino nucleic acid (ABA), a bridged nucleic acid (BNA), and/or a peptide nucleic acid (PNA).

In some aspects, the miRNA inhibitors described herein comprise a backbone modification. In certain aspects, the backbone modification is a phosphorodiamidate morpholino oligomer (PMO) and/or phosphorothioate (PS) modification.

In some aspects, the miRNA inhibitors described herein are delivered in a delivery agent. In certain aspects, the delivery agent is a micelle, an exosome, a lipid nanoparticle, an extracellular vesicle, or a synthetic vesicle.

In some aspects, the miRNA inhibitors described herein are delivered by a viral vector. In certain aspects, the viral vector is an AAV, an adenovirus, a retrovirus, or a lentivirus. In some aspects, the viral vector is an AAV that has a serotype of AAV2, AAV3, AAV4, AAV5, AAV6, AAV7, AAV8, AAV9, AAV10, or any combination thereof.

In some aspects, the miRNA inhibitors described herein are delivered with a delivery agent. In certain aspects, the delivery agent comprises a micelle, an exosome, a lipidoid, a liposome, a lipoplex, a lipid nanoparticle, an extracellular vesicle, a synthetic vesicle, a polymeric compound, a peptide, a protein, a cell, a nanoparticle mimic, a nanotube, a conjugate, a viral vector, or combinations thereof.

In some aspects, the delivery agent comprises a cationic carrier unit comprising

[WP]-L1-[CC]-L2-[AM] (formula I)

or

[WP]-L1-[AM]-L2-[CC] (formula II)
wherein
WP is a water-soluble biopolymer moiety;
CC is a positively charged carrier moiety;
AM is an adjuvant moiety; and,
L1 and L2 are independently optional linkers, and
wherein when mixed with a nucleic acid at an ionic ratio of about 1:1, the cationic carrier unit forms a micelle.

In some aspects, the miRNA inhibitors described herein interact with the cationic carrier unit via an ionic bond. In some aspects, the water-soluble polymer comprises poly(alkylene glycols), poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyglycerol, polyphosphazene, polyoxazolines (“POZ”) poly(N-acryloylmorpholine), or any combinations thereof. In other aspects, the water-soluble polymer comprises polyethylene glycol (“PEG”), polyglycerol, or poly(propylene glycol) (“PPG”).

In some aspects, the water-soluble polymer comprises:

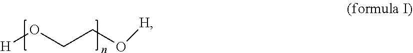

In some aspects, n is 1-1000. In certain aspects, the n is at least about 110, at least about 111, at least about 112, at least about 113, at least about 114, at least about 115, at least about 116, at least about 117, at least about 118, at least about 119, at least about 120, at least about 121, at least about 122, at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, or at least about 141. In further aspects, the n is about 80 to about 90, about 90 to about 100, about 100 to about 110, about 110 to about 120, about 120 to about 130, about 140 to about 150, about 150 to about 160.

In some aspects, the water-soluble polymer is linear, branched, or dendritic.

In some aspects, the cationic carrier moiety comprises one or more basic amino acids. In certain aspects, the cationic carrier moiety comprises at least three, at least four, at least five, at least six, at least seven, at least eight, at least nine, at least ten, at least 11, at least 12, at least 13, at least 14, at last 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 basic amino acids. In certain aspects, the cationic carrier moiety comprises about 30 to about 50 basic amino acids.

In some aspects, the basic amino acid comprises arginine, lysine, histidine, or any combination thereof. In some aspects, the cationic carrier moiety comprises about 40 lysine monomers.

In some aspects, the adjuvant moiety is capable of modulating an immune response, an inflammatory response, and/or a tissue microenvironment. In certain aspects, the adjuvant moiety comprises an imidazole derivative, an amino acid, a vitamin, or any combination thereof.

In some aspects, the adjuvant moiety comprises:

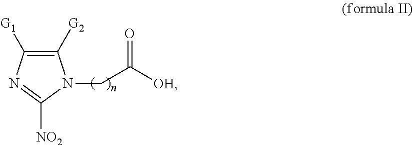

wherein each of G1 and G2 is H, an aromatic ring, or 1-10 alkyl, or G1 and G2 together form an aromatic ring, and wherein n is 1-10.

In some aspects, the adjuvant moiety comprises nitroimidazole. In certain aspects, the adjuvant moiety comprises metronidazole, tinidazole, nimorazole, dimetridazole, pretomanid, ornidazole, megazol, azanidazole, benznidazole, or any combination thereof.

In some aspects, the adjuvant moiety comprises an amino acid.

In some aspects, the adjuvant moiety comprises

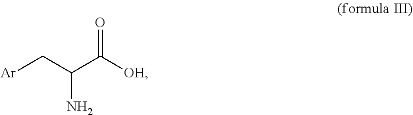

wherein Ar is

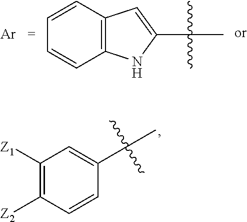

and
wherein each of Z1 and Z2 is H or OH.

In some aspects, the adjuvant moiety comprises a vitamin. In certain aspects, the vitamin comprises a cyclic ring or cyclic hetero atom ring and a carboxyl group or hydroxyl group.

In some aspects, the vitamin comprises:

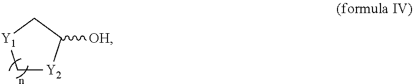

wherein each of Y1 and Y2 is C, N, O, or S, and wherein n is 1 or 2.

In some aspects, the vitamin is selected from the group consisting of vitamin A, vitamin B1, vitamin B2, vitamin B3, vitamin B6, vitamin B7, vitamin B9, vitamin B12, vitamin C, vitamin D2, vitamin D3, vitamin E, vitamin M, vitamin H, and any combination thereof. For example, the vitamin can be vitamin B3.

In some aspects, the adjuvant moiety comprises at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, or at least about 20 vitamin B3. In certain aspects, the adjuvant moiety comprises about 10 vitamin B3.

In some aspects, the delivery agent comprises about a water-soluble biopolymer moiety with about 120 to about 130 PEG units, a cationic carrier moiety comprising a poly-lysine with about 30 to about 40 lysines, and an adjuvant moiety with about 5 to about 10 vitamin B3.

In some aspects, the delivery agent is associated with the miRNA inhibitor, thereby forming a micelle. For example, the association can be a covalent bond, a non-covalent bond, or an ionic bond.

In some aspects, the cationic carrier unit and the miRNA inhibitor in the micelle is mixed in a solution so that the ionic ratio of the positive charges of the cationic carrier unit and the negative charges of the miRNA inhibitor is about 1:1. In some aspects, the cationic carrier unit is capable of protecting the miRNA inhibitor from enzymatic degradation.

In some aspects, the miRNA inhibitors described herein are administered parenthetically, intramuscularly, subcutaneously, ophthalmic, intravenously, intraperitoneally, intradermally, intraorbitally, intracerebrally, intracranially, intracerebroventricularly, intraspinally, intraventricular, intrathecally, intracistemally, intracapsularly, intratumorally, or any combination thereof.

In some aspects, the miRNA inhibitors described herein are administered to a skin area where promoting hair growth is needed by spread, spray, steam, or injection.

In some aspects, the miRNA inhibitors described herein are administered topically to a skin area where promoting hair growth is needed.

In some aspects, the miRNA inhibitors described herein are formulated in a form selected from the group consisting of an ointment, a shampoo, a conditioner, a lotion, a tonic, a gel, and a mousse. In some aspects, the administering step is performed by soaking or bathing the subject in the miRNA inhibitor described herein formulated in a form selected from the group consisting of an ointment, a shampoo, a conditioner, a lotion, a tonic, a gel, and a mousse.

### BRIEF DESCRIPTION OF THE DRAWINGS/FIGURES

FIG. 1 shows an exemplary architecture of a carrier unit of the present disclosure. The example presented includes a cationic carrier moiety, which can interact electrostatically with a miRNA inhibitor (antimirs). In some aspects, tissue specific adjuvant moiety (AM) can be located between water-soluble biopolymer (WP) and cationic carrier (CC). The CC and AM components are portrayed in a linear arrangement for simplicity. However, CC and AM can be arranged in a scaffold fashion.

FIGS. 2A-2F provide evaluation of the hair growth effect of a miR-485 inhibitor (485 ASO-001) on depilated C57BL/6J mice 5 days (FIG. 2A), 7 days (FIG. 2B), 9 days (FIG. 2C), 12 days (FIG. 2D), 14 days (FIG. 2E), and 16 days (FIG. 2F) after administration of 0.1 mg/kg (mpk), 0.3 mg/kg (mpk) or 0.6 mg/kg (mpk) of the miR-485 inhibitor (485 ASO-001), or phosphate-buffered saline (PBS) (negative control).

FIG. 2G shows the hair regrowth quantification in depilated C57BL/6J mice 5 days, 7 days, 9 days, 12 days, and 14 days after administration of 0.1 mg/kg (mpk), 0.3 mg/kg (mpk) or 0.6 mg/kg (mpk) of miR-485 inhibitor (485 ASO-001), or phosphate-buffered saline (PBS) (negative control). The y-axis shows hair regrowth quantification scale (0-6), where grade 0=skin pink, no pigmentation; grade 1=<30% skin area showing the darkening, but no visible hair; grade 2=30%-70% skin area showing the darkening, no visible hair; grade 3=>70% skin area showing the darkening, or 30% hair visible; grade 4=>70% skin area showing the darkening, and 30% -70% hair visible; grade 5=>70% skin area showing the darkening, and >70% hair visible; and grade 6=>90% skin area showing darkening, >90% and hair visible. Data are expressed as the mean values (n=6)±standard error of the mean (SEM). *p<0.05 and **p<0.01 of miR485-3p inhibitor (485 ASO-001)-treated groups compared with PBS-treated controls.

FIGS. 3A-3B show the effect of miR-485 inhibitor (485 ASO-001) on the hair shaft density in depilated C57BL/6J mice 12 days (FIG. 3A) and 16 days (FIG. 3B) after administration of 0.1 mg/kg (mpk), 0.3 mg/kg (mpk) or 0.6 mg/kg (mpk) of miR-485 inhibitor (485 ASO-001), or phosphate-buffered saline (PBS) (negative control).

FIG. 3C shows the hair density quantification in depilated C57BL/6J mice 12 (white bars) and 16 days (shaded bars) after administration of 0.1 mg/kg (mpk), 0.3 mg/kg (mpk) or 0.6 mg/kg (mpk) of miR-485 inhibitor (485 ASO-001), or phosphate-buffered saline (PBS) (negative control). The y-axis shows the evaluation of hair density by analyzing the images (×200 magnification; actual area, 3.6 mm²). Data are expressed as the mean values (n=6)±standard error of the mean (SEM). *p<0.05 and **p<0.01 of 485 ASO-001-treated groups compared with PBS-treated controls.

FIGS. 4A-4B show the effect of miR-485 inhibitor (485 ASO-001) on the hair length in depilated C57BL/6J mice 16 days (FIG. 4A) and 21 days (FIG. 4B) after administration of 0.1 mg/kg (mpk), 0.3 mg/kg (mpk) or 0.6 mg/kg (mpk) of miR-485 inhibitor (485 ASO-001), or phosphate-buffered saline (PBS) (negative control).

FIG. 4C shows the hair length quantification in depilated C57BL/6J mice 16 (white bars) and 21 days (shaded bars) after administration of 0.1 mg/kg (mpk), 0.3 mg/kg (mpk) or 0.6 mg/kg (mpk) of miR-485 inhibitor (485 ASO-001), or phosphate-buffered saline (PBS) (negative control). The y-axis shows the hair length in mm. Data are expressed as the mean values (n=6)±standard error of the mean (SEM). *p<0.05 and **p<0.01 of 485 ASO-001-treated groups compared with PBS-treated controls.

FIGS. 4D-4I provide evaluation of the hair regrowth effect of the miR-485 inhibitor (485 ASO-001) on C57BL/6J mice 5 days (FIG. 4D), 7 days (FIG. 4E), 10 days (FIG. 4F), 12 days (FIG. 4G), 14 days (FIG. 4H), and 16 days (FIG. 4I) after administration of twice intramuscular injection 0.3 mg/kg (mpk) or 0.6 mg/kg (mpk) of miR-485 inhibitor (485 ASO-001), or phosphate-buffered saline (PBS) (negative control) and positive control (2% Minoxidil).

FIG. 4J shows the hair regrowth quantification in depilated C57BL/6J mice 7 days, 10 days, 12 days, 14 days, 16 days after twice intramuscular injection of 0.3 mg/kg (mpk) or 0.6 mg/kg (mpk) of miR-485 inhibitor (485 ASO-001), or phosphate-buffered saline (PBS) (negative control) and positive control Minoxidil. The y-axis shows hair regrowth quantification scale (0-6), where grade 0=skin pink, no pigmentation; grade 1=<30% skin area showing the darkening, but no visible hair; grade 2=30%-70% skin area showing the darkening, no visible hair; grade 3=>70% skin area showing the darkening, or 30% hair visible; grade 4=>70% skin area showing the darkening, and 30% -70% hair visible; grade 5=>70% skin area showing the darkening, and >70% hair visible; and grade 6=>90% skin area showing darkening, >90% and hair visible. Data are based on 5 mice per group and are reported as a mean±SEM.

FIGS. 5A-5B show the effect of miR-485 inhibitor (485 ASO-001) on the 485-3p (FW7_mimic) expression level on day 10 post-depilation (PD) (FIG. 5A) and 16 days (FIG. 5B) after administration of twice intramuscular injection of 0.3 mg/kg (mpk) (middle bar) or 0.6 mg/kg (mpk) (right bar) of miR-485 inhibitor (485 ASO-001 or “ASO”) or phosphate-buffered saline (PBS) (negative control) (left bar).

FIGS. 6A-6B show H&E staining results of mice dorsal skin during the hair follicle cycle 10 post depilation (pd). FIG. 6A shows a longitudinal and transverse section of mouse dorsal skin treated with phosphate-buffered saline (PBS) (negative control) and positive control (Minoxidil) after 10 days of treatment. FIG. 6B shows a longitudinal and transverse section of mice dorsal skin treated with twice intramuscular injection of 0.3 mg/kg (mpk) and 0.6 mg/kg (mpk) of miR-485 inhibitor (485 ASO-001) after 10 days of treatment.

FIGS. 6C-6H show the effect of miR-485 inhibitor (485 ASO-001) on the thickness of dermis (μm) (FIG. 6C) and subcutis (μm) (FIG. 6D), hair follicle length (μm) (FIG. 6E) and hair bulb diameter (μm) (FIG. 6F), hair follicle density in dermis (FIG. 6G) and subcutis (FIG. 6H), in depilated C57BL/6J mice on 10 pd. The different treatment groups are the same as that described in FIGS. 6A and 6B.

FIGS. 7A-7B show H&E staining results of mice dorsal skin on 16 pd. FIG. 7A shows a longitudinal and transverse section of mouse dorsal skin treated with phosphate-buffered saline (PBS) (negative control) and positive control (Minoxidil) after 16 days of treatment. FIG. 7B shows a longitudinal and transverse section of mouse dorsal skin treated with twice intramuscular injection of 0.3 mg/kg (mpk) and 0.6 mg/kg (mpk) of miR-485 inhibitor (485 ASO-001) after 16 days of treatment.

FIGS. 7C-7H show the effect of miR-485 inhibitor (485 ASO-001) on the thickness of subcutis (μm) (FIG. 7C) and dermis (μm) (FIG. 7D), hair follicle length (μm) (FIG. 7E) and hair bulb diameter (μm) (FIG. 7F), hair follicle density in dermis (FIG. 7G) and subcutis (FIG. 7H), in depilated C57BL/6J mice on 16 Pd after twice Intramuscular injection of 0.3 mg/kg (mpk) or 0.6 mg/kg (mpk) of miR-485 inhibitor (485 ASO-001), or phosphate-buffered saline (PBS) (negative control) and (Minoxidil positive control) on 16 pd. n=3-10 mice per time point.

FIG. 8A shows Western blotting analysis of the expression of CD36 and vascular endothelial growth factor (VEGF-A).

FIGS. 8B-8C show CD36 protein (FIG. 8B) or VEGF-A protein (FIG. 8C) expression quantification on 10 pd after treatment with twice intramuscular injection of 0.6 mg/kg (mpk) of miR-485 inhibitor (485 ASO-001) or phosphate-buffered saline (PBS) (negative control). Relative expression of proteins was normalized using β-actin. Values are mean±standard error of the mean. *p<0.05; **p<0.01; *** p<0.001.

FIG. 9A shows Western blotting analysis of the expression of Wnt3a and β-catenin.

FIGS. 9B-9C show Wnt3a protein (FIG. 9B) or β-catenin protein (FIG. 9C) expression quantification on 10 pd after treatment with twice intramuscular injection of 0.6 mg/kg (mpk) of miR-485 inhibitor (485 ASO-001) or phosphate-buffered saline (PBS) (negative control). Relative expression of proteins was normalized using β-actin. Values are mean±standard error of the mean. *p<0.05; **p<0.01; *** p<0.001.

FIGS. 10A-10B show immunofluorescent staining for CD36. PBS treated control (FIG. 10A) and CD36-enriched dermal sheath (DS) expressing are observed in perivascular regions of hair follicle in miR-485 inhibitor (485 ASO-001) treated hair follicles (FIG. 10B) Scale bar=50 μm.

### DETAILED DESCRIPTION OF THE DISCLOSURE

The present disclosure is directed to the use of a miR-485 inhibitor for inducing hair growth, increasing hair density, increasing the follicular density, increasing the hair shaft thickness, increasing hair length, preventing hair loss, reducing hair loss, or any combination thereof in a subject in need thereof, comprising a nucleotide sequence encoding a nucleotide molecule that comprises at least one miR-485 binding site, wherein the nucleotide molecule does not encode a protein.

Before the present disclosure is described in greater detail, it is to be understood that this disclosure is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present disclosure. Any recited method can be carried out in the order of events recited or in any other order which is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “hair” is used herein to mean scalp, head, facial and/or body hair, including but not limited to the scalp, eye lashes, brows, mustache, beard, ear, nasal, chest, pubic, auxiliary, and the like.

The term “hair growth” is used herein to mean earlier inducing growth of a new hair cycle, prolonging the active growth phase (anagen) of the hair cycle, increasing the growth rate of the hair, and/or increasing the width of hair shaft, including, but not limited to, the induction of the growth of hair and making it more visible to the eye.

The terms “hair loss” and “hair thinning” are used herein to mean a decrease in normal hair density and/or shortening of the normal growth phase (anagen) of the hair cycle and/or reduction of the width of hair shaft, and reduction of the number of hairs, which can be caused by age increase, genetically predisposed and/or other causes, and can be suffered by male or female, young or old. The terms “hair loss,” “alopecia,” “balding,” and “pattern hair loss” are used interchangeably herein.

The term “induce hair growth,” “promote hair growth,” “stimulate hair growth,” “facilitate hair growth,” or “increase hair growth,” is used herein to mean at least one of the results of an increase in number and/or length and/or thickness of hair on at least part of the affected skin (or scalp) surface.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, ‘a’ represents adenine, ‘c’ represents cytosine, ‘g’ represents guanine, ‘t’ represents thymine, and ‘u’ represents uracil.

Amino acid sequences are written left to right in amino to carboxy orientation. Amino acids are referred to herein by either their commonly known three letter symbols or by the one-letter symbols recommended by the IUPAC-IUB Biochemical Nomenclature Commission.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAVrh.74, snake AAV, avian AAV, bovine AAV, canine AAV, equine AAV, ovine AAV, goat AAV, shrimp AAV, those AAV serotypes and clades disclosed by Gao et al. (J. Virol. 78:6381 (2004)) and Moris et al. (Virol. 33:375 (2004)), and any other AAV now known or later discovered. See, e.g., FIELDS et al. VIROLOGY, volume 2, chapter 69 (4th ed., Lippincott-Raven Publishers). In some aspects, an “AAV” includes a derivative of a known AAV. In some aspects, an “AAV” includes a modified or an artificial AAV.

The terms “administration,” “administering,” and grammatical variants thereof refer to introducing a composition, such as a miRNA inhibitor of the present disclosure, into a subject via a pharmaceutically acceptable route. The introduction of a composition, such as a micelle comprising a miRNA inhibitor of the present disclosure, into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. Administration includes self-administration and the administration by another. A suitable route of administration allows the composition or the agent to perform its intended function. For example, if a suitable route is intravenous, the composition is administered by introducing the composition or agent into a vein of the subject.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are at least 70% identical, at least 80% identical, at least 90% identical, or at least 95% identical to one another. In some aspects, two or more sequences are said to be “highly conserved” if they are about 70% identical, about 80% identical, about 90% identical, about 95%, about 98%, or about 99% identical to one another. In some aspects, two or more sequences are said to be “conserved” if they are at least 30% identical, at least 40% identical, at least 50% identical, at least 60% identical, at least 70% identical, at least 80% identical, at least 90% identical, or at least 95% identical to one another. In some aspects, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence can apply to the entire length of a polynucleotide or polypeptide or can apply to a portion, region or feature thereof.

The term “derived from,” as used herein, refers to a component that is isolated from or made using a specified molecule or organism, or information (e.g., amino acid or nucleic acid sequence) from the specified molecule or organism. For example, a nucleic acid sequence that is derived from a second nucleic acid sequence can include a nucleotide sequence that is identical or substantially similar to the nucleotide sequence of the second nucleic acid sequence. In the case of nucleotides or polypeptides, the derived species can be obtained by, for example, naturally occurring mutagenesis, artificial directed mutagenesis or artificial random mutagenesis. The mutagenesis used to derive nucleotides or polypeptides can be intentionally directed or intentionally random, or a mixture of each. The mutagenesis of a nucleotide or polypeptide to create a different nucleotide or polypeptide derived from the first can be a random event (e.g., caused by polymerase infidelity) and the identification of the derived nucleotide or polypeptide can be made by appropriate screening methods, e.g., as discussed herein. In some aspects, a nucleotide or amino acid sequence that is derived from a second nucleotide or amino acid sequence has a sequence identity of at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% to the second nucleotide or amino acid sequence, respectively, wherein the first nucleotide or amino acid sequence retains the biological activity of the second nucleotide or amino acid sequence.

As used herein, a “coding region” or “coding sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide.

The terms “complementary” and “complementarity” refer to two or more oligomers (i.e., each comprising a nucleobase sequence), or between an oligomer and a target gene, that are related with one another by Watson-Crick base-pairing rules. For example, the nucleobase sequence “T-G-A (5′→3′),” is complementary to the nucleobase sequence “A-C-T (3′→5′).” Complementarity can be “partial,” in which less than all of the nucleobases of a given nucleobase sequence are matched to the other nucleobase sequence according to base pairing rules. For example, in some aspects, complementarity between a given nucleobase sequence and the other nucleobase sequence can be about 70%, about 75%, about 80%, about 85%, about 90%, or about 95%. Accordingly, in certain aspects, the term “complementary” refers to at least about 80%, at least about 85%, at least about 90%, at least about 91%, at least about 92%, at least about 93%, at least about 94%, at least about 95%, at least about 96%, at least about 97%, at least about 98%, or at least about 99% match or complementarity to a target nucleic acid sequence (e.g., miR-485 nucleic acid sequence). Or, there can be “complete” or “perfect” (100%) complementarity between a given nucleobase sequence and the other nucleobase sequence to continue the example. In some aspects, the degree of complementarity between nucleobase sequences has significant effects on the efficiency and strength of hybridization between the sequences.

The term “downstream” refers to a nucleotide sequence that is located 3′ to a reference nucleotide sequence. In certain aspects, downstream nucleotide sequences relate to sequences that follow the starting point of transcription. For example, the translation initiation codon of a gene is located downstream of the start site of transcription.

The terms “excipient” and “carrier” are used interchangeably and refer to an inert substance added to a pharmaceutical composition to further facilitate administration of a compound, e.g., a miRNA inhibitor of the present disclosure.

The term “expression,” as used herein, refers to a process by which a polynucleotide produces a gene product, e.g., RNA or a polypeptide. It includes without limitation transcription of the polynucleotide into micro RNA binding site, small hairpin RNA (shRNA), small interfering RNA (siRNA), or any other RNA product. It includes, without limitation, transcription of the polynucleotide into messenger RNA (mRNA), and the translation of mRNA into a polypeptide. Expression produces a “gene product.” As used herein, a gene product can be, e.g., a nucleic acid, such as an RNA produced by transcription of a gene. As used herein, a gene product can be either a nucleic acid, RNA or miRNA produced by the transcription of a gene, or a polypeptide which is translated from a transcript. Gene products described herein further include nucleic acids with post transcriptional modifications, e.g., polyadenylation or splicing, or polypeptides with post translational modifications, e.g., phosphorylation, methylation, glycosylation, the addition of lipids, association with other protein subunits, or proteolytic cleavage.

As used herein, the term “homology” refers to the overall relatedness between polymeric molecules, e.g. between nucleic acid molecules. Generally, the term “homology” implies an evolutionary relationship between two molecules. Thus, two molecules that are homologous will have a common evolutionary ancestor. In the context of the present disclosure, the term homology encompasses both to identity and similarity.

In some aspects, polymeric molecules are considered to be “homologous” to one another if at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of the monomers in the molecule are identical (exactly the same monomer) or are similar (conservative substitutions). The term “homologous” necessarily refers to a comparison between at least two sequences (e.g., polynucleotide sequences).

In the context of the present disclosure, substitutions (even when they are referred to as amino acid substitution) are conducted at the nucleic acid level, i.e., substituting an amino acid residue with an alternative amino acid residue is conducted by substituting the codon encoding the first amino acid with a codon encoding the second amino acid.

As used herein, the term “identity” refers to the overall monomer conservation between polymeric molecules, e.g., between polynucleotide molecules. The term “identical” without any additional qualifiers, e.g., polynucleotide A is identical to polynucleotide B, implies the polynucleotide sequences are 100% identical (100% sequence identity). Describing two sequences as, e.g., “70% identical,” is equivalent to describing them as having, e.g., “70% sequence identity.”

Calculation of the percent identity of two polypeptide or polynucleotide sequences, for example, can be performed by aligning the two sequences for optimal comparison purposes (e.g., gaps can be introduced in one or both of a first and a second polypeptide or polynucleotide sequences for optimal alignment and non-identical sequences can be disregarded for comparison purposes). In certain aspects, the length of a sequence aligned for comparison purposes is at least 30%, at least 40%, at least 50%, at least 60%, at least 70%, at least 80%, at least 90%, at least 95%, or 100% of the length of the reference sequence. The amino acids at corresponding amino acid positions, or bases in the case of polynucleotides, are then compared.

When a position in the first sequence is occupied by the same amino acid or nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs that can be used to align different sequences (e.g., polynucleotide sequences) are available from various sources. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

As used herein, the terms “isolated,” “purified,” “extracted,” and grammatical variants thereof are used interchangeably and refer to the state of a preparation of desired composition of the present disclosure, e.g., a miRNA inhibitor of the present disclosure, that has undergone one or more processes of purification. In some aspects, isolating or purifying as used herein is the process of removing, partially removing (e.g., a fraction) of a composition of the present disclosure, e.g., a miRNA inhibitor of the present disclosure from a sample containing contaminants.

In some aspects, an isolated composition has no detectable undesired activity or, alternatively, the level or amount of the undesired activity is at or below an acceptable level or amount. In other aspects, an isolated composition has an amount and/or concentration of desired composition of the present disclosure, at or above an acceptable amount and/or concentration and/or activity. In other aspects, the isolated composition is enriched as compared to the starting material from which the composition is obtained. This enrichment can be by at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.9%, at least about 99.99%, at least about 99.999%, at least about 99.9999%, or greater than 99.9999% as compared to the starting material.

In some aspects, isolated preparations are substantially free of residual biological products. In some aspects, the isolated preparations are 100% free, at least about 99% free, at least about 98% free, at least about 97% free, at least about 96% free, at least about 95% free, at least about 94% free, at least about 93% free, at least about 92% free, at least about 91% free, or at least about 90% free of any contaminating biological matter. Residual biological products can include abiotic materials (including chemicals) or unwanted nucleic acids, proteins, lipids, or metabolites.

The term “linked” as used herein refers to a first amino acid sequence or polynucleotide sequence covalently or non-covalently joined to a second amino acid sequence or polynucleotide sequence, respectively. The first amino acid or polynucleotide sequence can be directly joined or juxtaposed to the second amino acid or polynucleotide sequence or alternatively an intervening sequence can covalently join the first sequence to the second sequence. The term “linked” means not only a fusion of a first polynucleotide sequence to a second polynucleotide sequence at the 5′-end or the 3′-end, but also includes insertion of the whole first polynucleotide sequence (or the second polynucleotide sequence) into any two nucleotides in the second polynucleotide sequence (or the first polynucleotide sequence, respectively). The first polynucleotide sequence can be linked to a second polynucleotide sequence by a phosphodiester bond or a linker. The linker can be, e.g., a polynucleotide.

A “miRNA inhibitor,” as used herein, refers to a compound that can decrease, alter, and/or modulate miRNA expression, function, and/or activity. The miRNA inhibitor can be a polynucleotide sequence that is at least partially complementary to the target miRNA nucleic acid sequence, such that the miRNA inhibitor hybridizes to the target miRNA sequence. For instance, in some aspects, a miR-485 inhibitor of the present disclosure comprises a nucleotide sequence encoding a nucleotide molecule that is at least partially complementary to the target miR-485 (e.g., miR-485-3p) nucleic acid sequence, such that the miR-485 inhibitor hybridizes to the miR-485 sequence. In further aspects, the hybridization of the miR-485 to the miR-485 inhibitor sequence decreases, alters, and/or modulates the expression, function, and/or activity of miR-485 (e.g., hybridization results in an increase in the expression of SIRT1 protein and/or SIRT1 gene).

The terms “miRNA,” “miR,” and “microRNA” are used interchangeably and refer to a microRNA molecule found in eukaryotes that is involved in RNA-based gene regulation. The term will be used to refer to the single-stranded RNA molecule processed from a precursor. In some aspects, the term “antisense oligomers” can also be used to describe the microRNA molecules of the present disclosure. Names of miRNAs and their sequences related to the present disclosure are provided herein. MicroRNAs recognize and bind to target mRNAs through imperfect base pairing leading to destabilization or translational inhibition of the target mRNA and thereby downregulate target gene expression. Conversely, targeting miRNAs via molecules comprising a miRNA binding site (generally a molecule comprising a sequence complementary to the seed region of the miRNA) can reduce or inhibit the miRNA-induced translational inhibition leading to an upregulation of the target gene.

The terms “mismatch” or “mismatches” refer to one or more nucleobases (whether contiguous or separate) in an oligomer nucleobase sequence (e.g., miR-485 inhibitor) that are not matched to a target nucleic acid sequence (e.g., miR-485) according to base pairing rules. While perfect complementarity is often desired, in some aspects, one or more (e.g., 6, 5, 4, 3, 2, or 1 mismatches) can occur with respect to the target nucleic acid sequence. Variations at any location within the oligomer are included. In certain aspects, antisense oligomers of the disclosure (e.g., miR-485 inhibitor) include variations in nucleobase sequence near the termini, variations in the interior, and if present are typically within about 6, 5, 4, 3, 2, or 1 subunit of the 5′ and/or 3′ terminus. In some aspects, one, two, or three nucleobases can be removed and still provide on-target binding.

As used herein, the terms “modulate,” “modify,” and grammatical variants thereof, generally refer when applied to a specific concentration, level, expression, function or behavior, to the ability to alter, by increasing or decreasing, e.g., directly or indirectly promoting/stimulating/up-regulating or interfering with/inhibiting/down-regulating the specific concentration, level, expression, function or behavior, such as, e.g., to act as an antagonist or agonist. In some instances, a modulator can increase and/or decrease a certain concentration, level, activity or function relative to a control, or relative to the average level of activity that would generally be expected or relative to a control level of activity. In some aspects, a miRNA inhibitor disclosed herein, e.g., a miR-485 inhibitor, can modulate (e.g., decrease, alter, or abolish) miR-485 expression, function, and/or activity, and thereby, modulate SIRT1 protein or gene expression and/or activity.

“Nucleic acid,” “nucleic acid molecule,” “nucleotide sequence,” “polynucleotide,” and grammatical variants thereof are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein.

The terms “pharmaceutically acceptable carrier,” “pharmaceutically acceptable excipient,” and grammatical variations thereof, encompass any of the agents approved by a regulatory agency of the U.S. Federal government or listed in the U.S. Pharmacopeia for use in animals, including humans, as well as any carrier or diluent that does not cause the production of undesirable physiological effects to a degree that prohibits administration of the composition to a subject and does not abrogate the biological activity and properties of the administered compound. Included are excipients and carriers that are useful in preparing a pharmaceutical composition and are generally safe, non-toxic, and desirable.

As used herein, the term “pharmaceutical composition” refers to one or more of the compounds described herein, such as, e.g., a miRNA inhibitor of the present disclosure, mixed or intermingled with, or suspended in one or more other chemical components, such as pharmaceutically acceptable carriers and excipients. One purpose of a pharmaceutical composition is to facilitate administration of preparations comprising a miRNA inhibitor of the present disclosure to a subject.

The term “polynucleotide,” as used herein, refers to polymers of nucleotides of any length, including ribonucleotides, deoxyribonucleotides, analogs thereof, or mixtures thereof.

In some aspects, the term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide.

In some aspects, the term “polynucleotide” includes polydeoxyribonucleotides (containing 2-deoxy-D-ribose), polyribonucleotides (containing D-ribose), including tRNA, rRNA, shRNA, siRNA, miRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA.

In some aspects of the present disclosure, a polynucleotide can be, e.g., an oligonucleotide, such as an antisense oligonucleotide. In some aspects, the oligonucleotide is an RNA. In some aspects, the RNA is a synthetic RNA. In some aspects, the synthetic RNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine).

The terms “polypeptide,” “peptide,” and “protein” are used interchangeably herein to refer to polymers of amino acids of any length, e.g., that are encoded by the SIRT1 gene. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art. The term “polypeptide,” as used herein, refers to proteins, polypeptides, and peptides of any size, structure, or function.

Polypeptides include gene products, naturally occurring polypeptides, synthetic polypeptides, homologs, orthologs, paralogs, fragments and other equivalents, variants, and analogs of the foregoing.

A polypeptide can be a single polypeptide or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to about 50 amino acids long, e.g., about 5, about 10, about 15, about 20, about 25, about 30, about 35, about 40, about 45, or about 50 amino acids long.

The terms “prevent,” “preventing,” and variants thereof as used herein, refer partially or completely delaying onset of an disease, disorder and/or condition; partially or completely delaying onset of one or more symptoms, features, or clinical manifestations of a particular disease, disorder, and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition. In some aspects, preventing an outcome is achieved through prophylactic treatment.

The terms “prevent hair loss,” “preventing hair loss,” “prevent hair thinning,” and “preventing hair thinning” as used herein mean an effect on decreasing any hair loss or hair thinning as described herein in advance.

The terms “reduce hair loss,” “reducing hair loss,” “reduce hair thinning,” and “reducing hair thinning” as used herein mean an effect on decreasing any hair loss or hair thinning as described herein in a subject in need thereof (e.g., in a subject affected by hair loss).

As used herein, the terms “promoter” and “promoter sequence” are interchangeable and refer to a DNA sequence capable of controlling the expression of a coding sequence or functional RNA. In general, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity.

The promoter sequence is typically bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase. In some aspects, a promoter that can be used with the present disclosure includes a tissue specific promoter.

As used herein, “prophylactic” refers to a therapeutic or course of action used to prevent the onset of a disease or condition, or to prevent or delay a symptom associated with a disease or condition.

As used herein, a “prophylaxis” refers to a measure taken to maintain health and prevent the onset of a disease or condition, or to prevent or delay a symptom associated with a disease or condition.

As used herein, the term “gene regulatory region” or “regulatory region” refers to nucleotide sequences located upstream (5′ non-coding sequences), within, or downstream (3′ non-coding sequences) of a coding region, and which influence the transcription, RNA processing, stability, or translation of the associated coding region. Regulatory regions can include promoters, translation leader sequences, introns, polyadenylation recognition sequences, RNA processing sites, effector binding sites, or stem-loop structures. If a coding region is intended for expression in a eukaryotic cell, a polyadenylation signal and transcription termination sequence will usually be located 3′ to the coding sequence.

In some aspects, a miR-485 inhibitor disclosed herein (e.g., a polynucleotide comprising a RNA comprising one or more miR-485 binding site) can include a promoter and/or other expression (e.g., transcription) control elements operably associated with one or more coding regions. In an operable association a coding region for a gene product is associated with one or more regulatory regions in such a way as to place expression of the gene product under the influence or control of the regulatory region(s). For example, a coding region and a promoter are “operably associated” if induction of promoter function results in the transcription of mRNA encoding the gene product encoded by the coding region, and if the nature of the linkage between the promoter and the coding region does not interfere with the ability of the promoter to direct the expression of the gene product or interfere with the ability of the DNA template to be transcribed. Other expression control elements, besides a promoter, for example enhancers, operators, repressors, and transcription termination signals, can also be operably associated with a coding region to direct gene product expression.

As used herein, the term “similarity” refers to the overall relatedness between polymeric molecules, e.g. between polynucleotide molecules (e.g. miRNA molecules). Calculation of percent similarity of polymeric molecules to one another can be performed in the same manner as a calculation of percent identity, except that calculation of percent similarity takes into account conservative substitutions as is understood in the art. It is understood that percentage of similarity is contingent on the comparison scale used, i.e., whether the nucleic acids are compared, e.g., according to their evolutionary proximity, charge, volume, flexibility, polarity, hydrophobicity, aromaticity, isoelectric point, antigenicity, or combinations thereof.

The terms “subject,” “patient,” “individual,” and “host,” and variants thereof are used interchangeably herein and refer to any mammalian subject, including without limitation, humans, domestic animals (e.g., dogs, cats and the like), farm animals (e.g., cows, sheep, pigs, horses and the like), and laboratory animals (e.g., monkey, rats, mice, rabbits, guinea pigs and the like) for whom diagnosis, treatment, or therapy is desired, particularly humans. The methods described herein are applicable to both human therapy and veterinary applications.

As used herein, the phrases “subject in need thereof” and “subject at risk of hair loss” include subjects, such as mammalian subjects, that would benefit from administration of a miRNA inhibitor of the disclosure (e.g., miR-485 inhibitor), e.g., to induce hair growth. In some aspects, the subject has one or more disorders selected from the group consisting of alopecia greata, androgenic alopecia, alopecia areata, alopecia universalis, involutional alopecia, trichotillomania, telogen effluvium, anagen effluvium, cicatricial, alopecia, scarring alopecia, scalp thinning, hair shaft abnormalities, infectious hair disorders, genetic disorders, and hair loss due to chemotherapy, hormonal imbalance, fungal infection, medication intake, chemical hair treatment, or aging. In some aspects, the subject is a human.

As used herein, the term “therapeutically effective amount” is the amount of reagent or pharmaceutical compound comprising a miRNA inhibitor of the present disclosure that is sufficient to a produce a desired therapeutic effect, pharmacologic and/or physiologic effect on a subject in need thereof. A therapeutically effective amount can be a “prophylactically effective amount” as prophylaxis can be considered therapy.

The terms “treat,” “treatment,” or “treating,” as used herein refers to, e.g., the reduction in severity of a disease or condition; the reduction in the duration of a disease course; the amelioration or elimination of one or more symptoms associated with a disease or condition (e.g., diabetes); the provision of beneficial effects to a subject with a disease or condition, without necessarily curing the disease or condition. The term also includes prophylaxis or prevention of a disease or condition or its symptoms thereof.

The term “upstream” refers to a nucleotide sequence that is located 5′ to a reference nucleotide sequence.

A “vector” refers to any vehicle for the cloning of and/or transfer of a nucleic acid into a host cell. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “vector” includes both viral and nonviral vehicles for introducing the nucleic acid into a cell in vitro, ex vivo or in vivo. A large number of vectors are known and used in the art including, for example, plasmids, modified eukaryotic viruses, or modified bacterial viruses. Insertion of a polynucleotide into a suitable vector can be accomplished by ligating the appropriate polynucleotide fragments into a chosen vector that has complementary cohesive termini.

Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), β-galactosidase (LacZ), β-glucuronidase (Gus), and the like. Selectable markers can also be considered to be reporters.

### II. Methods of Use

Provided herein are the methods of inducing hair growth in a subject in need thereof (e.g., a subject at risk of hair loss) comprising administering to the subject a compound that inhibits miR-485 (“miRNA inhibitor”). Also provided herein are the methods for reducing hair loss in a subject in need thereof (e.g., a subject at risk of hair loss) comprising administering to the subject a compound that inhibits miR-485. Also provided herein are the methods of increasing hair density in a subject in need thereof (e.g., a subject at risk of hair loss) comprising administering to the subject a compound that inhibits miR-485. Also provided herein are the methods of increasing the hair length in a subject in need thereof (e.g., a subject at risk of hair loss) comprising administering to the subject a compound that inhibits miR-485. Also provided herein are the methods of increasing the follicular density in a subject in need thereof (e.g., a subject at risk of hair loss) comprising administering to the subject a compound that inhibits miR-485. Also provided herein are the methods of increasing the hair shaft thickness in a subject in need thereof (e.g., a subject at risk of hair loss) comprising administering to the subject a compound that inhibits miR-485. Also provided herein are the methods for preventing hair loss in a subject at risk of hair loss comprising administering to the subject a compound that inhibits miR-485 (miRNA inhibitor).

The underlying cause of the hair loss is not always the same from individual to individual. Also, the process by which hair grows encompasses several phases and there are many contributory factors that can alter the normal vigorous growth of hair. The hair growth cycle is divided into three phases: an anagen phase, in which the hair is growing actively, with a very substantial level of cell proliferation occurring in the hair follicle; a catagen phase, when the follicle slows down its proliferative activity temporarily to permit hair development; and a telogen phase, in which the follicle simply stops growing and regresses, until the hair is shed, and a new anagen phase begins. It is of course completely normal for the average person to lose many hairs on a daily basis, and therefore, this cycle is normally repeated continually throughout life, to replenish the hair that is lost. The cycle does slow down with age in all individuals, however with the normal hairs gradually being replaced by progressively finer hair (vellus hair), and the cycles becoming shorter. Early-to-mid stage hair loss is almost entirely manifested as thinning of the hair shafts of a large percentage of follicles and retarded shaft growth so that the hairs that are present, are thin, light and close to the scalp, while follicle density and shafts per follicle are basically unchanged. For individuals who suffer from abnormal hair loss, it is apparent that the normal cyclical process becomes disrupted in some fashion, whether it be through an abnormal acceleration or other alteration of the process. This eventually results in a more rapid shift to the telogen phase, which in turn gradually results in the production of more vellus hair and ultimately can result in baldness. Only at end-stage baldness do the follicles become irreversibly inactive. See e.g., Int'l Publ. No. WO2004/078117A2.

Hair thickness or density D can be quantified as the product of three factors: (1) follicular density, F, measured in follicles per square centimeter, (2) average number of hair shafts per follicle, N, and (3) average thickness, T, of the hair shafts. The product of these three quantities that indicates hair density D.

D=F*N*T

Another possible objective measure of hair thinning can be the fraction of hair widths outside two standard deviations for a selected subset of hair from the head.

In another alternative measure of hair density, hair thickness (diameter) can be replaced with hair cross sectional area A=πT²/4. The overall sense or indicator of “fullness” of a head of hair can also include the hair length L, resulting in a hair volume parameter V=F*N*A*L. Since one can increase the sense of hair volume by letting remaining hair grow, V serves as an appearance metric but masks hair thinning. Thus, D=FNA will be the objective standard used.

Typical follicular unit densities are in the range of 60-120 cm⁻² and each follicle generally contains one or two shafts, but rarely, more than two hair shafts of varying ages. The hair shaft thickness can be classified as coarse, medium, or fine, and the mean value of the shaft thickness will vary from about 40 microns in width for fine hair, while coarse hair might average 90 microns in width. N will generally be a number between 1 and 2, and more commonly 1-1.25 it can be eliminated from the density determination but may need to be considered in some rare cases.

A normalized hair density measurement for every individual is the ratio of top and/or front hair density to left and/or right side hair density. Thus, an individual's hair thinning ratio can be expressed as: R=100×(Dₜₒₚ)/D_{side}, with Dₜₒₚ and D_{side}=F*N*T for each respective region. See e.g., U.S. Publ. No. 2009/0036800, which is incorporated herein by reference in its entirety.

In some aspects, the subject has one or more disorders selected from the group consisting of alopecia greata, androgenic alopecia, alopecia areata, alopecia universalis, involutional alopecia, trichotillomania, telogen effluvium, anagen effluvium, cicatricial, alopecia, scarring alopecia, scalp thinning, hair shaft abnormalities, infectious hair disorders, genetic disorders, and hair loss due to chemotherapy, hormonal imbalance, fungal infection, medication intake, chemical hair treatment, or aging. In some aspects, the subject is a human.

In some aspects, a miR-485 inhibitor disclosed herein can be administered by any suitable route known in the art. In some aspects, a miR-485 inhibitor is administered parenthetically, intramuscularly, subcutaneously, ophthalmic, intravenously, intraperitoneally, intradermally, intraorbitally, intracerebrally, intracranially, intracerebroventricularly, intraspinally, intraventricular, intrathecally, intraci stemally, intracapsularly, intratumorally, topically, or any combination thereof. In certain aspects, a miR-485 inhibitor is administered intramuscularly.

In some aspects, a miR-485 inhibitor is administered to a skin area where promoting hair growth is needed by spread, spray, steam, or injection.

In some aspects, a miR-485 inhibitor is administered topically to a skin area where promoting hair growth is needed.

In some aspects, a miR-485 inhibitor is formulated in a form selected from the group consisting of an ointment, a shampoo, a conditioner, a lotion, a tonic, a gel, and a mousse. In some aspects, the administering step is performed by soaking or bathing the subject in the miRNA inhibitor formulated in a form selected from the group consisting of an ointment, a shampoo, a conditioner, a lotion, a tonic, a gel, and a mousse.

In some aspects, a miR-485 inhibitor of the present disclosure can be used in combination with one or more additional therapeutic agents. In some aspects, the additional therapeutic agent and the miR-485 inhibitor are administered concurrently. In certain aspects, the additional therapeutic agent and the miR-485 inhibitor are administered sequentially.

In some aspects, the administration of a miR-485 inhibitor disclosed herein does not result in any adverse effects. In certain aspects, the miR-485 inhibitors of the present disclosure do not adversely affect body weight when administered to a subject. In some aspects, the miR-485 inhibitors disclosed herein do not result in increased mortality or cause pathological abnormalities when administered to a subject.

In some aspects, the miR-485 inhibitors of the present disclosure can exert therapeutic effects (e.g., inducing hair growth or preventing hair loss) by regulating the expression and/or activity of one or more genes. In some aspects, miR-485 inhibitors disclosed herein are capable of regulating the expression and/or activity of a gene selected from CTBP1, TRIP6, SIRT1, CD36, PGC1-a, or combinations thereof.

### CTBP1 Regulation

In some aspects, contacting a miR-485 inhibitor described herein with a cell can increase the expression of a CTBP1 protein and/or a CTBP1 gene in the cell.

C-terminal-binding protein 1 (CTBP1) is a protein that in humans is encoded by the CTBP1 gene. CTBP1 is a regulatory protein that binds to sequence-specific DNA-binding proteins and help turn genes off, e.g., by recruiting histone modifying enzymes that add repressive histone marks and remove activating marks. CTBP1 protein can also self-associate and bring together gene regulatory complexes.

In humans, the CTBP1 gene is located on chromosome 4 (nucleotides 1,211,444 to 1,250,355 of GenBank Accession Number NC_000004.12, minus strand orientation). Synonyms of the CTBP1 gene, and the encoded protein thereof, are known and include “Brefeldin A-Ribosylated Substrate,” “BARS,” “HADDTS,” CtBP1,” or “CTBP.”

There are at least two known isoforms of human CTBP1 protein, resulting from alternative splicing. CTBP1 isoform 1 (UniProt identifier: Q13363-1; SEQ ID NO: 31) consists of 440 amino acids and has been chosen as the canonical sequence. CTBP1 isoform 2 (UniProt identifier: Q13363-2; SEQ ID NO: 32) consists of 429 amino acids and differs from the canonical sequence as follows: 1-13: MGSSHLLNKGLPL→MS. Table 1 below provides the sequences for the two CTBP1 isoforms.

TABLE 1
CTBP1 Protein Isoforms

| | |
|---|---|
| Isoform 1 | MGSSHLLNKGLPLGVRPPIMNGPLHPRPLVALLDGRDC |
| (UniProt: | TVEMPILKDVATVAFCDAQSTQEIHEKVLNEAVGALMY |
| Q13363-1) | HTITLTREDLEKFKALRIIVRIGSGFDNIDIKSAGDLG |
| (SEQ ID NO: | IAVCNVPAASVEETADSTLCHILNLYRRATWLHQALRE |
| 31) | GTRVQSVEQIREVASGAARIRGETLGIIGLGRVGQAVA |
| | LRAKAFGFNVLFYDPYLSDGVERALGLQRVSTLQDLLF |
| | HSDCVTLHCGLNEHNHHLINDFTVKQMRQGAFLVNTAR |
| | GGLVDEKALAQALKEGRIRGAALDVHESEPFSFSQGPL |
| | KDAPNLICTPHAAWYSEQASIEMREEAAREIRRAITGR |
| | IPDSLKNCVNKDHLTAATHWASMDPAVVHPELNGAAYR |
| | YPPGVVGVAPTGIPAAVEGIVPSAMSLSHGLPPVAHPP |
| | HAPSPGQTVKPEADRDHASDQL |
| Isoform 2 | MSGVRPPIMNGPLHPRPLVALLDGRDCTVEMPILKDVA |
| (UniProt: | TVAFCDAQSTQEIHEKVLNEAVGALMYHTITLTREDLE |
| Q13363-2) | KFKALRIIVRIGSGFDNIDIKSAGDLGIAVCNVPAASV |
| (SEQ ID NO: | EETADSTLCHILNLYRRATWLHQALREGTRVQSVEQIR |
| 32) | EVASGAARIRGETLGIIGLGRVGQAVALRAKAFGFNVL |
| | FYDPYLSDGVERALGLQRVSTLQDLLFHSDCVTLHCGL |
| | NEHNHHLINDFTVKQMRQGAFLVNTARGGLVDEKALAQ |
| | ALKEGRIRGAALDVHESEPFSFSQGPLKDAPNLICTPH |
| | AAWYSEQASIEMREEAAREIRRAITGRIPDSLKNCVNK |
| | DHLTAATHWASMDPAVVHPELNGAAYRYPPGVVGVAPT |
| | GIPAAVEGIVPSAMSLSHGLPPVAHPPHAPSPGQTVKP |
| | EADRDHASDQL |

As used herein, the term “CTBP1” includes any variants or isoforms of CTBP1 which are naturally expressed by cells. Accordingly, in some aspects, a miR-485 inhibitor disclosed herein can increase the expression of CTBP1 isoform 1. In some aspects, a miR-485 inhibitor disclosed herein can increase the expression of CTBP1 isoform 2. In further aspects, a miR-485 inhibitor disclosed herein can increase the expression of both CTBP1 isoform 1 and isoform 2. Unless indicated otherwise, both isoform 1 and isoform 2 are collectively referred to herein as “CTBP1.”

In some aspects, contacting a cell with a miR-485 inhibitor increases the expression and/or activity of CTBP1 protein and/or CTBP1 gene in the cell by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 60-fold, at least about 70-fold, at least about 80-fold, at least about 90-fold, or at least about 100-fold or more, compared to the expression and/or activity in a reference cell (e.g., corresponding cell that has not been contacted with the miR-485 inhibitor).

Not to be bound by any one theory, in some aspects, a miR-485 inhibitor disclosed herein increases the expression of CTBP1 protein and/or CTBP1 gene by reducing the expression and/or activity of miR-485, e.g., miR-485-3p.

### TRIP6 Regulation

In some aspects, contacting a miR-485 inhibitor described herein with a cell can increase the expression of a TRIP6 protein and/or a TRIP6 gene in the cell.

Thyroid receptor-interacting protein 6 (TRIP6) is a protein that in humans is encoded by the TRIP6 gene. TRIP6 protein is a member of the zyxin family and comprises three LIM zinc-binding domains. TRIP6 protein has been shown to localize to focal adhesion sites and along actin stress fibers.

In humans, the TRIP6 gene is located on chromosome 7 (nucleotides 100,867,387 to 100,873,454 of GenBank Accession Number NC_000007.14, plus strand orientation). Synonyms of the TRIP6 gene, and the encoded protein thereof, are known and include: “Thyroid Hormone Receptor Interactor 6,” “ZRP-1,” “ OIP1, ” “Thyroid Hormone Receptor Interacting Protein 6,” “OPA-Interacting Protein 1,” and “Zyxin Related Protein 1.”

There are at least three known isoforms of human TRIP6 protein, resulting from alternative splicing. TRIP6 isoform 1 (UniProt identifier: Q15654-1; SEQ ID NO: 78) consists of 4766 amino acids and has been chosen as the canonical sequence. TRIP6 isoform 2 (UniProt identifier: Q15654-2; SEQ ID NO: 79) consists of 106 amino acids and differs from the canonical sequence as follows: (i) 37-106: ALQPHPRVINF . . . IDLLSSTLAE→VLPGPRGTGG . . . CVTATRPTGI; (ii) 107-476: Missing. TRIP6 isoform 3 (UniProt identifier: Q15654-2; SEQ ID NO: 80) consists of 80 amino acids and differs from the canonical sequence as follows: (i) 37-80: ALQPHPRVNF . . . SHGVLQHTQG→GAPCRQGGPS . . . CVTATRPTGI; (ii) 81-476: Missing. Table 2 below provides the sequences for the different TRIP6 protein isoforms.

TABLE 2
TRIP6 Protein Isoforms

| Isoform 1 | MSGPTWLPPKQPEPARAPQGRAIPRGTPGPPPAHG |
|---|---|
| (UniProt: | AALQPHPRVNFCPLPSEQCYQAPGGPEDRGPAWVG |
| Q15654-1) | SHGVLQHTQGLPADRGGLRPGSLDAEIDLLSSTLA |
| (SEQ ID NO: | ELNGGRGHASRRPDRQAYEPPPPPAYRTGSLKPNP |
| 78) | ASPLPASPYGGPTPASYTTASTPAGPAFPVQVKVA |
| | QPVRGCGPPRRGASQASGPLPGPHFPLPGRGEVWG |
| | PGYRSQREPGPGAKEEAAGVSGPAGRGRGGEHGPQ |
| | VPLSQPPEDELDRLTKKLVHDMNHPPSGEYFGQCG |
| | GCGEDVVGDGAGVVALDRVFHVGCFVCSTCRAQLR |
| | GQHFYAVERRAYCEGCYVATLEKCATCSQPILDRI |
| | LRAMGKAYHPGCFTCVVCHRGLDGIPFTVDATSQI |
| | HCIEDFHRKFAPRCSVCGGAIMPEPGQEETVRIVA |
| | LDRSFHIGCYKCEECGLLLSSEGECQGCYPLDGHI |
| | LCKACSAWRIQELSATVTTDC |
| Isoform 2 | SGPTWLPPKQPEPARAPQGRAIPRGTPGPPPAHGA |
| (UniProt: | VLPGPRGTGGSGAGVGGVPWSTPAHAGAPCRQGGP |
| Q15654-2) | SPWKPGRRDRLAEQHAGRAEWGSGSCVTATRPTGI |
| (SEQ ID NO: | |
| 79) | |
| Isoform 3 | MSGPTWLPPKQPEPARAPQGRAIPRGTPGPPPAHG |
| (UniProt: | AGAPCRQGGPSPWKPGRRDRLAEQHAGRAEWGSGS |
| Q15654-2) | CVTATRPTGI |
| (SEQ ID NO: | |
| 80) | |

As used herein, the term “TRIP6” includes any variants or isoforms of TRIP6 which are naturally expressed by cells. Accordingly, in some aspects, a miR-485 inhibitor disclosed herein can increase the expression of TRIP6 isoform 1. In some aspects, a miR-485 inhibitor disclosed herein can increase the expression of TRIP6 isoform 2. In some aspects, a miR-485 inhibitor disclosed herein can increase the expression of TRIP6 isoform 3. In further aspects, a miR-485 inhibitor disclosed herein can increase the expression of both TRIP6 isoform 1, isoform 2, and isoform 3. Unless indicated otherwise, isoform 1, isoform, 2, and isoform 3 are collectively referred to herein as “TRIP6.”

In some aspects, contacting a cell with a miR-485 inhibitor increases the expression and/or activity of TRIP6 protein and/or TRIP6 gene in the cell by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 60-fold, at least about 70-fold, at least about 80-fold, at least about 90-fold, or at least about 100-fold or more, compared to the expression and/or activity in a reference cell (e.g., corresponding cell that has not been contacted with the miR-485 inhibitor).

In some aspects, a miR-485 inhibitor can also increase the expression and/or activity of other LIM-domain containing proteins.

Not to be bound by any one theory, in some aspects, a miR-485 inhibitor disclosed herein increases the expression of TRIP6 protein and/or TRIP6 gene by reducing the expression and/or activity of miR-485, e.g., miR-485-3p.

### SIRT1 Regulation

In some aspects, contacting a miR-485 inhibitor described herein with a cell (e.g., neural stem cells) can increase the expression of a SIRT1 protein and/or a SIRT1 gene in the cell.

Sirtuin 1 (SIRT1), also known as NAD-dependent deacetylase sirtuin-1, is a protein that in humans is encoded by the SIRT1 gene. The SIRT1 gene is located on chromosome 10 in humans (nucleotides 67,884,656 to 67,918,390 of GenBank Accession Number NC_000010.11, plus strand orientation). Synonyms of the SIRT1 gene, and the encoded protein thereof, are known and include “regulatory protein SIR2 homolog 1,” “silent mating-type information regulation 2 homolog 1,” “SIR2,” “SIR2-Like Protein 1,” “SIR2L1,” “SIR2alpha,” “Sirtuin Type 1,” “hSIRT1,” or “hSIR2.”

There are at least two known isoforms of human SIRT1 protein, resulting from alternative splicing. SIRT1 isoform 1 (UniProt identifier: Q96EB6-1) consists of 747 amino acids and has been chosen as the canonical sequence (SEQ ID NO: 31). SIRT1 isoform 2 (also known as “delta-exon8) (UniProt identifier: Q96EB6-2) consists of 561 amino acids and differs from the canonical sequence as follows: 454-639: missing (SEQ ID NO: 32). Table 3 below provides the sequences for the two SIRT1 isoforms.

TABLE 3
SIRT1 Protein Isoforms

| | |
|---|---|
| Isoform 1 | MADEAALALQPGGSPSAAGADREAASSPAGEPL |
| (UniProt: | RKRPRRDGPGLERSPGEPGGAAPEREVPAAARG |
| Q96EB6-1) | CPGAAAAALWREAEAEAAAAGGEQEAQATAAAG |
| (SEQ ID NO: | EGDNGPGLQGPSREPPLADNLYDEDDDDEGEEE |
| 31) | EEAAAAAIGYRDNLLFGDEIITNGFHSCESDEE |
| | DRASHASSSDWTPRPRIGPYTFVQQHLMIGTDP |
| | RTILKDLLPETIPPPELDDMTLWQIVINILSEP |
| | PKRKKRKDINTIEDAVKLLQECKKIIVLTGAGV |
| | SVSCGIPDFRSRDGIYARLAVDFPDLPDPQAMF |
| | DIEYFRKDPRPFFKFAKEIYPGQFQPSLCHKFI |
| | ALSDKEGKLLRNYTQNIDTLEQVAGIQRIIQCH |
| | GSFATASCLICKYKVDCEAVRGDIFNQVVPRCP |
| | RCPADEPLAIMKPEIVFFGENLPEQFHRAMKYD |
| | KDEVDLLIVIGSSLKVRPVALIPSSIPHEVPQI |
| | LINREPLPHLHFDVELLGDCDVIINELCHRLGG |
| | EYAKLCCNPVKLSEITEKPPRTQKELAYLSELP |
| | PTPLHVSEDSSSPERTSPPDSSVIVTLLDQAAK |
| | SNDDLDVSESKGCMEEKPQEVQTSRNVESIAEQ |
| | MENPDLKNVGSSTGEKNERTSVAGTVRKCWPNR |
| | VAKEQISRRLDGNQYLFLPPNRYIFHGAEVYSD |
| | SEDDVLSSSSCGSNSDSGTCQSPSLEEPMEDES |
| | EIEEFYNGLEDEPDVPERAGGAGFGTDGDDQEA |
| | INEAISVKQEVTDMNYPSNKS |
| Isoform 2 | MADEAALALQPGGSPSAAGADREAASSPAGEPL |
| (UniProt: | RKRPRRDGPGLERSPGEPGGAAPEREVPAAARG |
| Q96EB6-2) | CPGAAAAALWREAEAEAAAAGGEQEAQATAAAG |
| (SEQ ID NO: | EGDNGPGLQGPSREPPLADNLYDEDDDDEGEEE |
| 32) | EEAAAAAIGYRDNLLFGDEIITNGFHSCESDEE |
| | DRASHASSSDWTPRPRIGPYTFVQQHLMIGTDP |
| | RTILKDLLPETIPPPELDDMTLWQIVINILSEP |
| | PKRKKRKDINTIEDAVKLLQECKKIIVLTGAGV |
| | SVSCGIPDFRSRDGIYARLAVDFPDLPDPQAMF |
| | DIEYFRKDPRPFFKFAKEIYPGQFQPSLCHKFI |
| | ALSDKEGKLLRNYTQNIDTLEQVAGIQRIIQCH |
| | GSFATASCLICKYKVDCEAVRGDIFNQVVPRCP |
| | RCPADEPLAIMKPEIVFFGENLPEQFHRAMKYD |
| | KDEVDLLIVIGSSLKVRPVALIPSNQYLFLPPN |
| | RYIFHGAEVYSDSEDDVLSSSSCGSNSDSGTCQ |
| | SPSLEEPMEDESEIEEFYNGLEDEPDVPERAGG |
| | AGFGTDGDDQEAINEAISVKQEVTDMNYPSNKS |

As used herein, the term “SIRT1” includes any variants or isoforms of SIRT1 which are naturally expressed by cells. Accordingly, in some aspects, a miR-485 inhibitor disclosed herein can increase the expression of SIRT1 isoform 1. In some aspects, a miR-485 inhibitor disclosed herein can increase the expression of SIRT1 isoform 2. In further aspects, a miR-485 inhibitor disclosed herein can increase the expression of both SIRT1 isoform 1 and isoform 2. Unless indicated otherwise, both isoform 1 and isoform 2 are collectively referred to herein as “SIRT1.”

In some aspects, contacting a cell with a miR-485 inhibitor increases the expression and/or activity of SIRT1 protein and/or SIRT1 gene in the cell by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 60-fold, at least about 70-fold, at least about 80-fold, at least about 90-fold, or at least about 100-fold or more, compared to the expression and/or activity in a reference cell (e.g., corresponding cell that has not been contacted with the miR-485 inhibitor).

Not to be bound by any one theory, in some aspects, a miR-485 inhibitor disclosed herein increases the expression of SIRT1 protein and/or SIRT1 gene by reducing the expression and/or activity of miR-485, e.g., miR-485-3p.

### CD36 Regulation

In some aspects, contacting a miR-485 inhibitor described herein with a cell can increase the expression of a CD36 protein and/or a CD36 gene in the cell.

Cluster determinant 36 (CD36) is also known as platelet glycoprotein 4, is a protein that in humans is encoded by the CD36 gene. The CD36 gene is located on chromosome 7 (nucleotides 80,602,656 to 80,679,277 of GenBank Accession Number NC_000007.14, plus strand orientation). Synonyms of the CD36 gene, and the encoded protein thereof, are known and include “platelet glycoprotein IV,” “fatty acid translocase,” “scavenger receptor class B member 3,” “glycoprotein 88,” “glycoprotein IIIb,” “glycoprotein IV,” “thrombospondin receptor,” “GPIIIB,” “PAS IV,” “GP3B,” “GPIV,” “FAT,” “GP4,” “BDPLT10,” “SCARB3,” “CHDS7,” “PASIV,” or “PAS-4.”

There are at least four known isoform of human CD36 protein, resulting from alternative splicing. CD36 isoform 1 (UniProt identifier: P16671-1) consists of 472 amino acids and has been chosen as the canonical sequence (SEQ ID NO: 36). CD36 isoform 2 (also known as “ex8-del”) (UniProt identifier: P16671-2) (SEQ ID NO: 37) consists of 288 amino acids and differs from the canonical sequence as follows: 274-288: SIYAVFESDVNLKGI→ETCVHFTSSFSVCKS; and 289-472: missing. CD36 Isoform 3 (also known as “ex6-7-del”) (UniProt identifier: P16671-3) (SEQ ID NO: 38) consists of 433 amino acids and differs from the canonical sequence as follows: 234-272: missing. CD36 isoform 4 (also known as “ex4-del” (UniProt identifier: P16671-4) (SEQ ID NO: 39) consists of 412 amino acids and differs from the canonical sequence as follows: 144-203: missing. Table 4 below provides the sequences for the four CD36 isoforms.

TABLE 4
CD36 Protein Isoforms

| | |
|---|---|
| Isoform 1 | MGCDRNCGLIAGAVIGAVLAVFGGILMPVG |
| (UniProt: | DLLIQKTIKKQVVLEEGTIAFKNWVKTGTE |
| P16671-1) | VYRQFWIFDVQNPQEVMMNSSNIQVKQRGP |
| (SEQ ID NO: | YTYRVRFLAKENVTQDAEDNTVSFLQPNGA |
| 36) | IFEPSLSVGTEADNFTVLNLAVAAASHIYQ |
| | NQFVQMILNSLINKSKSSMFQVRTLRELLW |
| | GYRDPFLSLVPYPVTTTVGLFYPYNNTADG |
| | VYKVFNGKDNISKVAIIDTYKGKRNLSYWE |
| | SHCDMINGTDAASFPPFVEKSQVLQFFSSD |
| | ICRSIYAVFESDVNLKGIPVYRFVLPSKAF |
| | ASPVENPDNYCFCTEKIISKNCTSYGVLDI |
| | SKCKEGRPVYISLPHFLYASPDVSEPIDGL |
| | NPNEEEHRTYLDIEPITGFTLQFAKRLQVN |
| | LLVKPSEKIQVLKNLKRNYIVPILWLNETG |
| | TIGDEKANMFRSQVTGKINLLGLIEMILLS |
| | VGVVMFVAFMISYCACRSKTIK |
| Isoform 2 | MGCDRNCGLIAGAVIGAVLAVFGGILMPVG |
| (UniProt: | DLLIQKTIKKQVVLEEGTIAFKNWVKTGTE |
| P16671-2) | VYRQFWIFDVQNPQEVMMNSSNIQVKQRGP |
| (SEQ ID NO: | YTYRVRFLAKENVTQDAEDNTVSFLQPNGA |
| 37) | IFEPSLSVGTEADNFTVLNLAVAAASHIYQ |
| | NQFVQMILNSLINKSKSSMFQVRTLRELLW |
| | GYRDPFLSLVPYPVTTTVGLFYPYNNTADG |
| | VYKVFNGKDNISKVAIIDTYKGKRNLSYWE |
| | SHCDMINGTDAASFPPFVEKSQVLQFFSSD |
| | ICRETCVHFTSSFSVCKS |
| Isoform 3 | MGCDRNCGLIAGAVIGAVLAVFGGILMPVG |
| (UniProt: | DLLIQKTIKKQVVLEEGTIAFKNWVKTGTE |
| P16671-3) | VYRQFWIFDVQNPQEVMMNSSNIQVKQRGP |
| (SEQ ID NO: | YTYRVRFLAKENVTQDAEDNTVSFLQPNGA |
| 38) | IFEPSLSVGTEADNFTVLNLAVAAASHIYQ |
| | NQFVQMILNSLINKSKSSMFQVRTLRELLW |
| | GYRDPFLSLVPYPVTTTVGLFYPYNNTADG |
| | VYKVFNGKDNISKVAIIDTYKGKRSIYAVF |
| | ESDVNLKGIPVYRFVLPSKAFASPVENPDN |
| | YCFCTEKIISKNCTSYGVLDISKCKEGRPV |
| | YISLPHFLYASPDVSEPIDGLNPNEEEHRT |
| | YLDIEPITGFTLQFAKRLQVNLLVKPSEKI |
| | QVLKNLKRNYIVPILWLNETGTIGDEKANM |
| | FRSQVTGKINLLGLIEMILLSVGVVMFVAF |
| | MISYCACRSKTIK |
| Isoform 4 | MGCDRNCGLIAGAVIGAVLAVFGGILMPVG |
| (UniProt: | DLLIQKTIKKQVVLEEGTIAFKNWVKTGTE |
| P16671-4) | VYRQFWIFDVQNPQEVMMNSSNIQVKQRGP |
| (SEQ ID NO: | YTYRVRFLAKENVTQDAEDNTVSFLQPNGA |
| 39) | IFEPSLSVGTEADNFTVLNLAVAYNNTADG |
| | VYKVFNGKDNISKVAIIDTYKGKRNLSYWE |
| | SHCDMINGTDAASFPPFVEKSQVLQFFSSD |
| | ICRSIYAVFESDVNLKGIPVYRFVLPSKAF |
| | ASPVENPDNYCFCTEKIISKNCTSYGVLDI |
| | SKCKEGRPVYISLPHFLYASPDVSEPIDGL |
| | NPNEEEHRTYLDIEPITGFTLQFAKRLQVN |
| | LLVKPSEKIQVLKNLKRNYIVPILWLNETG |
| | TIGDEKANMFRSQVTGKINLLGLIEMILLS |
| | VGVVMFVAFMISYCACRSKTIK |

As used herein, the term “CD36” includes any variants or isoforms of CD36 which are naturally expressed by cells. Accordingly, in some aspects, a miR-485 inhibitor disclosed herein can increase the expression of CD36 isoform 1. In some aspects, a miR-485 inhibitor disclosed herein can increase the expression of CD36 isoform 2. In some aspect, a miR-485 inhibitor disclosed herein can increase the expression of CD36 isoform 3. In some aspects, a miR-485 inhibitor disclosed herein can increase the expression of CD36 isoform 4. In further aspects, a miR-485 inhibitor disclosed herein can increase the expression of both CD36 isoform 1 and isoform 2, and/or isoform 3 and isoform 4, and/or isoform 1 and isoform 4, and/or isoform 2 and isoform 3. In some aspects, a miR-485 inhibitor disclosed herein can increase the expression of all CD36 isoforms. Unless indicated otherwise, isoform 1, isoform 2, isoform 3, and isoform 4 are collectively referred to herein as “CD36.”

In some aspects, contacting a cell with a miR-485 inhibitor increases the expression and/or activity of CD36 protein and/or CD36 gene in the cell by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 60-fold, at least about 70-fold, at least about 80-fold, at least about 90-fold, or at least about 100-fold or more, compared to the expression and/or activity in a reference cell (e.g., corresponding cell that has not been contacted with the miR-485 inhibitor).

Not to be bound by any one theory, in some aspects, a miR-485 inhibitor disclosed herein increases the expression of CD36 protein and/or CD36 gene by reducing the expression and/or activity of miR-485, e.g., miR-485-3p.

### PGC1 Regulation

In some aspects, contacting a miR-485 inhibitor described herein with a cell can increase the expression of a PGC-1α protein and/or a PGC-1α gene in the cell.

Peroxisome proliferator-activated receptor gamma coactivator 1-alpha (PGC1-α), also known as PPARG Coactivator 1 Alpha or Ligand Effect Modulator-6, is a protein that in humans is encoded by the PPARGC1A gene. The PGC1-α gene is located on chromosome 4 in humans (nucleotides 23,792,021 to 24,472,905 of GenBank Accession Number NC_000004.12, plus strand orientation). Synonyms of the PGC1-α gene, and the encoded protein thereof, are known and include “PPARGC1A,” “LEM6,” “PGC1,” “PGC1A,” “PGC-1v,” “PPARGC1, “PGC1alpha,” or “PGC-1(alpha).”

There are at least nine known isoforms of human PGC1-α protein, resulting from alternative splicing. PGC1-α isoform 1 (UniProt identifier: Q9UBK2-1) consists of 798 amino acids and has been chosen as the canonical sequence (SEQ ID NO: 40). PGC1-α isoform 2 (also known as “Isoform NT-7a”) (UniProt identifier: Q9UBK2-2) (SEQ ID NO: 41) consists of 271 amino acids and differs from the canonical sequence as follows: 269-271: DPK→LFL; 272-798: Missing. PGC1-α isoform 3 (also known as “Isoform B5”) (UniProt identifier: Q9UBK2-3) (SEQ ID NO: 42) consists of 803 amino acids and differs from the canonical sequence as follows: 1-18: MAWDMCNQDSESVWSDIE→MDETSPRLEEDWKKVLQREAGWQ. PGC1-α isoform 4 (also known as “Isoform B4”) (UniProt identifier: Q9UBK2-4) (SEQ ID NO: 43) consists of 786 amino acids and differs from the canonical sequence as follows: 1-18: MAWDMCNQDSESVWSDIE→MDEGYF. PGC1-α isoform 5 (also known as “Isoform B4-8a”) (UniProt identifier: Q9UBK2-5) (SEQ ID NO: 44) consists of 289 amino acids and differs from the canonical sequence as follows: 1-18: MAWDMCNQDSESVWSDIE→MDEGYF; 294-301: LTPPTTPP→VKTNLISK; 302-798: Missing. PGC1-α isoform 6 (also known as “Isoform B5-NT”) (UniProt identifier: Q9UBK2-6) (SEQ ID NO: 45) consists of 276 amino acids and differs from the canonical sequence as follows: 1-18: MAWDMCNQDSESVWSDIE→MDETSPRLEEDWKKVLQREAGWQ; 269-271: DPK LFL; 272-798: Missing. PGC1-α isoform 7 (also known as “B4-3ext”) (UniProt identifier: Q9UBK2-7) (SEQ ID NO: 46) consists of 138 amino acids and differs from the canonical sequence as follows: 1-18: MAWDMCNQDSESVWSDIE→MDEGYF; 144-150: LKKLLLA→VRTLPTV; 151-798: Missing. PGC1-α isoform 8 (also known as “Isoform 8a”) (UniProt identifier: Q9UBK2-8) (SEQ ID NO: 47) consists of 301 amino acids and differs from the canonical sequence as follows: 294-301: LTPPTTPP→VKTNLISK; 302-798: Missing. PGC1-α isoform 9 (also known as “Isoform 9” or “L-PGG-lalpha”) (UniProt identifier: Q9UBK2-9) (SEQ ID NO: 48) consists of 671 amino acids and differs from the canonical sequence as follows: 1-127: Missing. Table 5 below provides the sequences for the nine PGC1-α isoforms.

TABLE 5
PGC1-α Protein Isoforms

| | |
|---|---|
| Isoform 1 | MAWDMCNQDSESVWSDIECAALVGEDQPLC |
| (UniProt: | PDLPELDLSELDVNDLDTDSFLGGLKWCSD |
| Q9UBK2-1) | QSEIISNQYNNEPSNIFEKIDEENEANLLA |
| (SEQ ID NO: | VLTETLDSLPVDEDGLPSFDALTDGDVTTD |
| 40) | NEASPSSMPDGTPPPQEAEEPSLLKKLLLA |
| | PANTQLSYNECSGLSTQNHANHNHRIRTNP |
| | AIVKTENSWSNKAKSICQQQKPQRRPCSEL |
| | LKYLTTNDDPPHTKPTENRNSSRDKCTSKK |
| | KSHTQSQSQHLQAKPTTLSLPLTPESPNDP |
| | KGSPFENKTIERTLSVELSGTAGLTPPTTP |
| | PHKANQDNPFRASPKLKSSCKTVVPPPSKK |
| | PRYSESSGTQGNNSTKKGPEQSELYAQLSK |
| | SSVLTGGHEERKTKRPSLRLFGDHDYCQSI |
| | NSKTEILINISQELQDSRQLENKDVSSDWQ |
| | GQICSSTDSDQCYLRETLEASKQVSPCSTR |
| | KQLQDQEIRAELNKHFGHPSQAVFDDEADK |
| | TGELRDSDFSNEQFSKLPMFINSGLAMDGL |
| | FDDSEDESDKLSYPWDGTQSYSLFNVSPSC |
| | SSFNSPCRDSVSPPKSLFSQRPQRMRSRSR |
| | SFSRHRSCSRSPYSRSRSRSPGSRSSSRSC |
| | YYYESSHYRHRTHRNSPLYVRSRSRSPYSR |
| | RPRYDSYEEYQHERLKREEYRREYEKRESE |
| | RAKQRERQRQKAIEERRVIYVGKIRPDTTR |
| | TELRDRFEVFGEIEECTVNLRDDGDSYGFI |
| | TYRYTCDAFAALENGYTLRRSNETDFELYF |
| | CGRKQFFKSNYADLDSNSDDFDPASTKSKY |
| | DSLDFDSLLKEAQRSLRR |
| Isoform 2 | MAWDMCNQDSESVWSDIECAALVGEDQPLC |
| (UniProt: | PDLPELDLSELDVNDLDTDSFLGGLKWCSD |
| Q9UBK2-2) | QSEIISNQYNNEPSNIFEKIDEENEANLLA |
| (SEQ ID NO: | VLTETLDSLPVDEDGLPSFDALTDGDVTTD |
| 41) | NEASPSSMPDGTPPPQEAEEPSLLKKLLLA |
| | PANTQLSYNECSGLSTQNHANHNHRIRTNP |
| | AIVKTENSWSNKAKSICQQQKPQRRPCSEL |
| | LKYLTTNDDPPHTKPTENRNSSRDKCTSKK |
| | KSHTQSQSQHLQAKPTTLSLPLTPESPNLF |
| | L |
| Isoform 3 | MDETSPRLEEDWKKVLQREAGWQCAALVGE |
| (UniProt: | DQPLCPDLPELDLSELDVNDLDTDSFLGGL |
| Q9UBK2-3) | KWCSDQSEIISNQYNNEPSNIFEKIDEENE |
| (SEQ ID NO: | ANLLAVLTETLDSLPVDEDGLPSFDALTDG |
| 42) | DVTTDNEASPSSMPDGTPPPQEAEEPSLLK |
| | KLLLAPANTQLSYNECSGLSTQNHANHNHR |
| | IRTNPAIVKTENSWSNKAKSICQQQKPQRR |
| | PCSELLKYLTTNDDPPHTKPTENRNSSRDK |
| | CTSKKKSHTQSQSQHLQAKPTTLSLPLTPE |
| | SPNDPKGSPFENKTIERTLSVELSGTAGLT |
| | PPTTPPHKANQDNPFRASPKLKSSCKTVVP |
| | PPSKKPRYSESSGTQGNNSTKKGPEQSELY |
| | AQLSKSSVLTGGHEERKTKRPSLRLFGDHD |
| | YCQSINSKTEILINISQELQDSRQLENKDV |
| | SSDWQGQICSSTDSDQCYLRETLEASKQVS |
| | PCSTRKQLQDQEIRAELNKHFGHPSQAVFD |
| | DEADKTGELRDSDFSNEQFSKLPMFINSGL |
| | AMDGLFDDSEDESDKLSYPWDGTQSYSLFN |
| | VSPSCSSFNSPCRDSVSPPKSLFSQRPQRM |
| | RSRSRSFSRHRSCSRSPYSRSRSRSPGSRS |
| | SSRSCYYYESSHYRHRTHRNSPLYVRSRSR |
| | SPYSRRPRYDSYEEYQHERLKREEYRREYE |
| | KRESERAKQRERQRQKAIEERRVIYVGKIR |
| | PDTTRTELRDRFEVFGEIEECTVNLRDDGD |
| | SYGFITYRYTCDAFAALENGYTLRRSNETD |
| | FELYFCGRKQFFKSNYADLDSNSDDFDPAS |
| | TKSKYDSLDFDSLLKEAQRSLRR |
| Isoform 4 | MDEGYFCAALVGEDQPLCPDLPELDLSELD |
| (UniProt: | VNDLDTDSFLGGLKWCSDQSEIISNQYNNE |
| Q9UBK2-4) | PSNIFEKIDEENEANLLAVLTETLDSLPVD |
| (SEQ ID NO: | EDGLPSFDALTDGDVTTDNEASPSSMPDGT |
| 43) | PPPQEAEEPSLLKKLLLAPANTQLSYNECS |
| | GLSTQNHANHNHRIRTNPAIVKTENSWSNK |
| | AKSICQQQKPQRRPCSELLKYLTTNDDPPH |
| | TKPTENRNSSRDKCTSKKKSHTQSQSQHLQ |
| | AKPTTLSLPLTPESPNDPKGSPFENKTIER |
| | TLSVELSGTAGLTPPTTPPHKANQDNPFRA |
| | SPKLKSSCKTVVPPPSKKPRYSESSGTQGN |
| | NSTKKGPEQSELYAQLSKSSVLTGGHEERK |
| | TKRPSLRLFGDHDYCQSINSKTEILINISQ |
| | ELQDSRQLENKDVSSDWQGQICSSTDSDQC |
| | YLRETLEASKQVSPCSTRKQLQDQEIRAEL |
| | NKHFGHPSQAVFDDEADKTGELRDSDFSNE |
| | QFSKLPMFINSGLAMDGLFDDSEDESDKLS |
| | YPWDGTQSYSLFNVSPSCSSFNSPCRDSVS |
| | PPKSLFSQRPQRMRSRSRSFSRHRSCSRSP |
| | YSRSRSRSPGSRSSSRSCYYYESSHYRHRT |
| | HRNSPLYVRSRSRSPYSRRPRYDSYEEYQH |
| | ERLKREEYRREYEKRESERAKQRERQRQKA |
| | IEERRVIYVGKIRPDTTRTELRDRFEVFGE |
| | IEECTVNLRDDGDSYGFITYRYTCDAFAAL |
| | ENGYTLRRSNETDFELYFCGRKQFFKSNYA |
| | DLDSNSDDFDPASTKSKYDSLDFDSLLKEA |
| | QRSLRR |
| Isoform 5 | MDEGYFCAALVGEDQPLCPDLPELDLSELD |
| (UniProt: | VNDLDTDSFLGGLKWCSDQSEIISNQYNNE |
| Q9UBK2-5) | PSNIFEKIDEENEANLLAVLTETLDSLPVD |
| (SEQ ID NO: | EDGLPSFDALTDGDVTTDNEASPSSMPDGT |
| 44) | PPPQEAEEPSLLKKLLLAPANTQLSYNECS |
| | GLSTQNHANHNHRIRTNPAIVKTENSWSNK |
| | AKSICQQQKPQRRPCSELLKYLTTNDDPPH |
| | TKPTENRNSSRDKCTSKKKSHTQSQSQHLQ |
| | AKPTTLSLPLTPESPNDPKGSPFENKTIER |
| | TLSVELSGTAGVKTNLISK |
| Isoform 6 | MDETSPRLEEDWKKVLQREAGWQCAALVGE |
| (UniProt: | DQPLCPDLPELDLSELDVNDLDTDSFLGGL |
| Q9UBK2-6) | KWCSDQSEIISNQYNNEPSNIFEKIDEENE |
| (SEQ ID NO: | ANLLAVLTETLDSLPVDEDGLPSFDALTDG |
| 45) | DVTTDNEASPSSMPDGTPPPQEAEEPSLLK |
| | KLLLAPANTQLSYNECSGLSTQNHANHNHR |
| | IRTNPAIVKTENSWSNKAKSICQQQKPQRR |
| | PCSELLKYLTTNDDPPHTKPTENRNSSRDK |
| | CTSKKKSHTQSQSQHLQAKPTTLSLPLTPE |
| | SPNLFL |
| Isoform 7 | MDEGYFCAALVGEDQPLCPDLPELDLSELD |
| (UniProt: | VNDLDTDSFLGGLKWCSDQSEIISNQYNNE |
| Q9UBK2-7) | PSNIFEKIDEENEANLLAVLTETLDSLPVD |
| (SEQ ID NO: | EDGLPSFDALTDGDVTTDNEASPSSMPDGT |
| 46) | PPPQEAEEPSLVRTLPTV |
| Isoform 8 | MAWDMCNQDSESVWSDIECAALVGEDQPLC |
| (UniProt: | PDLPELDLSELDVNDLDTDSFLGGLKWCSD |
| Q9UBK2-8) | QSEIISNQYNNEPSNIFEKIDEENEANLLA |
| (SEQ ID NO: | VLTETLDSLPVDEDGLPSFDALTDGDVTTD |
| 47) | NEASPSSMPDGTPPPQEAEEPSLLKKLLLA |
| | PANTQLSYNECSGLSTQNHANHNHRIRTNP |
| | AIVKTENSWSNKAKSICQQQKPQRRPCSEL |
| | LKYLTTNDDPPHTKPTENRNSSRDKCTSKK |
| | KSHTQSQSQHLQAKPTTLSLPLTPESPNDP |
| | KGSPFENKTIERTLSVELSGTAGVKTNLIS |
| | K |
| Isoform 9 | MPDGTPPPQEAEEPSLLKKLLLAPANTQLS |
| (UniProt: | YNECSGLSTQNHANHNHRIRTNPAIVKTEN |
| Q9UBK2-9) | SWSNKAKSICQQQKPQRRPCSELLKYLTTN |
| (SEQ ID NO: | DDPPHTKPTENRNSSRDKCTSKKKSHTQSQ |
| 48) | SQHLQAKPTTLSLPLTPESPNDPKGSPFEN |
| | KTIERTLSVELSGTAGLTPPTTPPHKANQD |
| | NPFRASPKLKSSCKTVVPPPSKKPRYSESS |
| | GTQGNNSTKKGPEQSELYAQLSKSSVLTGG |
| | HEERKTKRPSLRLFGDHDYCQSINSKTEIL |
| | INISQELQDSRQLENKDVSSDWQGQICSST |
| | DSDQCYLRETLEASKQVSPCSTRKQLQDQE |
| | IRAELNKHFGHPSQAVFDDEADKTGELRDS |
| | DFSNEQFSKLPMFINSGLAMDGLFDDSEDE |
| | SDKLSYPWDGTQSYSLFNVSPSCSSFNSPC |
| | RDSVSPPKSLFSQRPQRMRSRSRSFSRHRS |
| | CSRSPYSRSRSRSPGSRSSSRSCYYYESSH |
| | YRHRTHRNSPLYVRSRSRSPYSRRPRYDSY |
| | EEYQHERLKREEYRREYEKRESERAKQRER |
| | QRQKAIEERRVIYVGKIRPDTTRTELRDRF |
| | EVFGEIEECTVNLRDDGDSYGFITYRYTCD |
| | AFAALENGYTLRRSNETDFELYFCGRKQFF |
| | KSNYADLDSNSDDFDPASTKSKYDSLDFDS |
| | LLKEAQRSLRR |

As used herein, the term “PGC1-α” includes any variants or isoforms of PGC1-α which are naturally expressed by cells. Accordingly, in some aspects, a miR-485 inhibitor disclosed herein can increase the expression of PGC1-α isoform 1. In some aspects, a miR-485 inhibitor disclosed herein can increase the expression of PGC1-α isoform 2. Accordingly, in some aspects, a miR-485 inhibitor disclosed herein can increase the expression of PGC1-α isoform 1. In some aspects, a miR-485 inhibitor disclosed herein can increase the expression of PGC1-α isoform 2. Accordingly, in some aspects, a miR-485 inhibitor disclosed herein can increase the expression of PGC1-α isoform 3. In some aspects, a miR-485 inhibitor disclosed herein can increase the expression of PGC1-α isoform 4. Accordingly, in some aspects, a miR-485 inhibitor disclosed herein can increase the expression of PGC1-α isoform 5. In some aspects, a miR-485 inhibitor disclosed herein can increase the expression of PGC1-α isoform 6. Accordingly, in some aspects, a miR-485 inhibitor disclosed herein can increase the expression of PGC1-α isoform 7. In some aspects, a miR-485 inhibitor disclosed herein can increase the expression of PGC1-α isoform 8. Accordingly, in some aspects, a miR-485 inhibitor disclosed herein can increase the expression of PGC1-α isoform 9. In further aspects, a miR-485 inhibitor disclosed herein can increase the expression of PGC1-α isoform 1, isoform 2, isoform 3, isoform 4, isoform 5, isoform 6, isoform 7, isoform 8, and isoform 9. Unless indicated otherwise, both isoform 1 and isoform 2 are collectively referred to herein as “PGC1-α.”

In some aspects, contacting a cell with a miR-485 inhibitor increases the expression and/or activity of PGC1-α protein and/or PGC1-α gene in the cell by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 60-fold, at least about 70-fold, at least about 80-fold, at least about 90-fold, or at least about 100-fold or more, compared to the expression and/or activity in a reference cell (e.g., corresponding cell that has not been contacted with the miR-485 inhibitor).

Not to be bound by any one theory, in some aspects, a miR-485 inhibitor disclosed herein increases the expression of PGC1-α protein and/or PGC1-α gene by reducing the expression and/or activity of miR-485, e.g., miR-485-3p.

### III. miRNA-485 Inhibitors Useful for the Present Disclosure

Disclosed herein are compounds that can inhibit miR-485 activity (miR-485 inhibitor). In some aspects, a miR-485 inhibitor of the present disclosure comprises a nucleotide sequence encoding or comprising a nucleotide molecule that comprises at least one miR-485 binding site, wherein the nucleotide molecule does not encode a protein. As described herein, in some aspects, the miR-485 binding site is at least partially complementary to the target miRNA nucleic acid sequence (i.e., miR-485), such that the miR-485 inhibitor hybridizes to the miR-485 nucleic acid sequence.

In some aspects, the miR-485 binding site of a miR inhibitor disclosed herein has at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence complementarity to the nucleic acid sequence of a miR-485, e.g., miR-485-3p. In certain aspects, the miR-485 binding site is fully complementary to the nucleic acid sequence of a miR-485, e.g., miR-485-3p.

The miR-485 hairpin precursor can generate both miR-485-5p and miR-485-3p. In the context of the present disclosure “miR-485” encompasses both miR-485-5p and miR-485-3p unless specified otherwise. The human mature miR-485-3p has the sequence 5′-GUCAUACACGGCUCUCCUCUCU-3′ (SEQ ID NO: 1; miRBase Acc. No. MIMAT0002176). A 5′ terminal subsequence of miR-485-3p 5′-UCAUACA-3′ (SEQ ID NO: 49) is the seed sequence. The human mature miR-485-5p has the sequence 5′-AGAGGCUGGCCGUGAUGAAUUC-3′ (SEQ ID NO: 33; miRBase Acc. No. MIMAT0002175). A 5′ terminal subsequence of miR-485-5p 5′-GAGGCUG-3′ (SEQ ID NO: 50) is the seed sequence.

As will be apparent to those in the art, the human mature miR-485-3p has significant sequence similarity to that of other species. For instance, the mouse mature miR-485-3p differs from the human mature miR-485-3p by a single amino acid at each of the 5′- and 3′- ends (i.e., has an extra “A” at the 5′-end and missing “C” at the 3′-end). The mouse mature miR-485-3p has the following sequence:

5′-AGUCAUACACGGCUCUCCUCUC-3′ (SEQ ID NO: 34;

miRBase Acc. No. MIMAT0003129; underlined portion corresponds to overlap to human mature miR-485-3p). The sequence for the mouse mature miR-485-5p is identical to that of the human: 5′-agaggcuggccgugaugaauuc-3′ (SEQ ID NO: 33; miRBase Acc. No. MIMAT0003128). In certain aspects, a miR-485 inhibitor disclosed herein is capable of binding to miR-485-3p and/or miR-485-5p from both human and mouse.

In some aspects, the miR-485 binding site is a single-stranded polynucleotide sequence that is complementary (e.g., fully complementary) to a sequence of a miR-485-3p (or a subsequence thereof). In some aspects, the miR-485-3p subsequence comprises the seed sequence. Accordingly, in certain aspects, the miR-485 binding site has at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence complementarity to the nucleic acid sequence set forth in SEQ ID NO: 49. In certain aspects, the miR-485 binding site is complementary to miR-485-3p except for 1, 2, 3, 4, 5, 6, 7, 8, 9, or 10 mismatches. In further aspects, the miR-485 binding site is fully complementary to the nucleic acid sequence set forth in SEQ ID NO: 1.

In some aspects, the miR-485 binding site is a single-stranded polynucleotide sequence that is complementary (e.g., fully complementary) to a sequence of a miR-485-5p (or a subsequence thereof). In some aspects, the miR-485-5p subsequence comprises the seed sequence. In certain aspects, the miR-485 binding site has at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence complementarity to the nucleic acid sequence set forth in SEQ ID NO: 50. In certain aspects, the miR-485 binding site is complementary to miR-485-5p except for 1, 2, 3, 4, 5, 6, 7, 8, 9, or 10 mismatches.

The seed region of a miRNA forms a tight duplex with the target mRNA. Most miRNAs imperfectly base-pair with the 3′ untranslated region (UTR) of target mRNAs, and the 5′ proximal “seed” region of miRNAs provides most of the pairing specificity. Without being bound to any theory, it is believed that the first nine miRNA nucleotides (encompassing the seed sequence) provide greater specificity whereas the miRNA ribonucleotides 3′ of this region allow for lower sequence specificity and thus tolerate a higher degree of mismatched base pairing, with positions 2-7 being the most important. Accordingly, in specific aspects of the present disclosure, the miR-485 binding site comprises a subsequence that is fully complementary (i.e., 100% complementary) over the entire length of the seed sequence of miR-485.

miRNA sequences and miRNA binding sequences (inhibitors) that can be used in the context of the disclosure include, but are not limited to, all or a portion of those sequences in the sequence listing provided herein, as well as the miRNA precursor sequence, or complement of one or more of these miRNAs. Any aspects of the disclosure involving specific miRNAs or miRNA binding sites by name are contemplated also to cover miRNAs or complementary sequences thereof whose sequences are at least about at least about 50%, at least about 55%, at least about 60%, at least about 65%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to the mature sequence of the specified miRNA sequence or complementary sequence thereof.

In some aspects, miRNA binding sequences of the present disclosure can include additional nucleotides at the 5′, 3′, or both 5′ and 3′ ends of those sequences in the sequence listing provided herein, as long as the modified sequence is still capable of specifically binding to miR-485. In some aspects, miRNA binding sequences of the present disclosure can differ in at least 1, 2, 3, 4, 5, 6, 7, 8, 9, 10 or more nucleotides with respect to those sequences in the sequence listing provided, as long as the modified sequence is still capable of specifically binding to miR-485.

It is also specifically contemplated that any methods and compositions discussed herein with respect to miRNA binding molecules or miRNA can be implemented with respect to synthetic miRNAs binding molecules. It is also understood that the disclosures related to RNA sequences in the present disclosure are equally applicable to corresponding DNA sequences.

In some aspects, a miRNA-485 inhibitor of the present disclosure comprises at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides at the 5′ of the nucleotide sequence. In some aspects, a miRNA-485 inhibitor comprises at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides at the 3′ of the nucleotide sequence.

In some aspects, a miR-485 inhibitor disclosed herein is about 6 to about 30 nucleotides in length. In certain aspects, a miR-485 inhibitor disclosed herein is 7 nucleotides in length. In further aspects, a miR-485 inhibitor disclosed herein is 8 nucleotides in length. In some aspects, a miR-485 inhibitor is 9 nucleotides in length. In some aspects, a miR-485 inhibitor of the present disclosure is 10 nucleotides in length. In certain aspects, a miR-485 inhibitor is 11 nucleotides in length. In further aspects, a miR-485 inhibitor is 12 nucleotides in length. In some aspects, a miR-485 inhibitor disclosed herein is 13 nucleotides in length. In certain aspects, a miR-485 inhibitor disclosed herein is 14 nucleotides in length. In some aspects, a miR-485 inhibitor disclosed herein is 15 nucleotides in length. In further aspects, a miR-485 inhibitor is 16 nucleotides in length. In certain aspects, a miR-485 inhibitor of the present disclosure is 17 nucleotides in length. In some aspects, a miR-485 inhibitor is 18 nucleotides in length. In some aspects, a miR-485 inhibitor is 19 nucleotides in length. In certain aspects, a miR-485 inhibitor is 20 nucleotides in length. In further aspects, a miR-485 inhibitor of the present disclosure is 21 nucleotides in length. In some aspects, a miR-485 inhibitor is 22 nucleotides in length.

In some aspects, a miR-485 inhibitor disclosed herein comprises a nucleotide sequence that is at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to a sequence selected from SEQ ID NOs: 2 to 30. In certain aspects, a miR-485 inhibitor comprises a nucleotide sequence selected from the group consisting of SEQ ID NOs: 2 to 30, wherein the nucleotide sequence can optionally comprise 1, 2, 3, 4, 5, 6, 7, 8, 9, or 10 mismatches.

In some aspects, a miRNA inhibitor comprises 5′-UGUAUGA-3′ (SEQ ID NO: 2), 5′-GUGUAUGA-3′ (SEQ ID NO: 3), 5′-CGUGUAUGA-3′ (SEQ ID NO: 4), 5′-CCGUGUAUGA-3′ (SEQ ID NO: 5), 5′-GCCGUGUAUGA-3′ (SEQ ID NO: 6), 5′-AGCCGUGUAUGA-3′ (SEQ ID NO: 7), 5′-GAGCCGUGUAUGA-3′ (SEQ ID NO: 8), 5′-AGAGCCGUGUAUGA-3′ (SEQ ID NO: 9), 5′-GAGAGCCGUGUAUGA-3′ (SEQ ID NO: 10), 5′-GGAGAGCCGUGUAUGA-3′ (SEQ ID NO: 11), 5′-AGGAGAGCCGUGUAUGA-3′ (SEQ ID NO: 12), 5′-GAGGAGAGCCGUGUAUGA-3′ (SEQ ID NO: 13), 5′-AGAGGAGAGCCGUGUAUGA-3′ (SEQ ID NO: 14), or 5′-GAGAGGAGAGCCGUGUAUGA-3′ (SEQ ID NO: 15).

In some aspects, the miRNA inhibitor has 5′-UGUAUGAC-3′ (SEQ ID NO: 16), 5′-GUGUAUGAC-3′ (SEQ ID NO: 17), 5′-CGUGUAUGAC-3′ (SEQ ID NO: 18), 5′-CCGUGUAUGAC-3′ (SEQ ID NO: 19), 5′-GCCGUGUAUGAC-3′ (SEQ ID NO: 20), 5′-AGCCGUGUAUGAC-3′ (SEQ ID NO: 21), 5′-GAGCCGUGUAUGAC-3′ (SEQ ID NO: 22), 5′-AGAGCCGUGUAUGAC-3′ (SEQ ID NO: 23), 5′-GAGAGCCGUGUAUGAC-3′ (SEQ ID NO: 24), 5′-GGAGAGCCGUGUAUGAC-3′ (SEQ ID NO: 25), 5′-AGGAGAGCCGUGUAUGAC-3′ (SEQ ID NO: 26), 5′-GAGGAGAGCCGUGUAUGAC-3′ (SEQ ID NO: 27), 5′-AGAGGAGAGCCGUGUAUGAC-3′ (SEQ ID NO: 28), or 5′-GAGAGGAGAGCCGUGUAUGAC-3′ (SEQ ID NO: 29).

In some aspects, the miRNA inhibitor has a sequence selected from the group consisting of: 5′-TGTATGA-3′ (SEQ ID NO: 30), 5′-GTGTATGA-3′ (SEQ ID NO: 51), 5′-CGTGTATGA-3′ (SEQ ID NO: 52), 5′-CCGTGTATGA-3′ (SEQ ID NO: 53), 5′-GCCGTGTATGA-3′ (SEQ ID NO: 54), 5′-AGCCGTGTATGA-3′ (SEQ ID NO: 55), 5′-GAGCCGTGTATGA-3′ (SEQ ID NO: 35), 5′-AGAGCCGTGTATGA-3′ (SEQ ID NO: 56), 5′-GAGAGCCGTGTATGA-3′ (SEQ ID NO: 57), 5′-GGAGAGCCGTGTATGA-3′ (SEQ ID NO: 58), 5′-AGGAGAGCCGTGTATGA-3′ (SEQ ID NO: 59), 5′-GAGGAGAGCCGTGTATGA-3′ (SEQ ID NO: 60), 5′-AGAGGAGAGCCGTGTATGA-3′ (SEQ ID NO: 61), 5′-GAGAGGAGAGCCGTGTATGA-3′ (SEQ ID NO: 62); 5′-TGTATGAC-3′ (SEQ ID NO: 63), 5′-GTGTATGAC-3′ (SEQ ID NO: 64), 5′-CGTGTATGAC-3′ (SEQ ID NO: 65), 5′-CCGTGTATGAC-3′ (SEQ ID NO: 66), 5′-GCCGTGTATGAC-3′ (SEQ ID NO: 67), 5′-AGCCGTGTATGAC-3′ (SEQ ID NO: 68),- 5′-GAGCCGTGTATGAC-3′ (SEQ ID NO: 69), 5′-AGAGCCGTGTATGAC-3′ (SEQ ID NO: 70), 5′-GAGAGCCGTGTATGAC-3′ (SEQ ID NO: 71), 5′-GGAGAGCCGTGTATGAC-3′ (SEQ ID NO: 72), 5′-AGGAGAGCCGTGTATGAC-3′ (SEQ ID NO: 73), 5′-GAGGAGAGCCGTGTATGAC-3′ (SEQ ID NO: 74), 5′-AGAGGAGAGCCGTGTATGAC-3′ (SEQ ID NO: 75), and 5′-GAGAGGAGAGCCGTGTATGAC-3′ (SEQ ID NO: 76).

In some aspects, a miRNA inhibitor disclosed herein (i.e., miR-485 inhibitor) comprises a nucleotide sequence that is at least about 50%, at least about 55%, at least about 60%, at least about 65%, at least about 70%, at least about 75%, at least about 80%, at least about 85%, at least about 90%, or at least about 95% identical to 5′-AGAGGAGAGCCGUGUAUGAC-3′ (SEQ ID NO: 28) or 5′-AGAGGAGAGCCGTGTATGAC-3′ (SEQ ID NO: 77). In some aspects, the miRNA inhibitor comprises a nucleotide sequence that has at least 90% similarity to 5′-AGAGGAGAGCCGUGUAUGAC-3′ (SEQ ID NO: 28) or 5′-AGAGGAGAGCCGTGTATGAC-3′ (SEQ ID NO: 77). In some aspects, the miRNA inhibitor comprises the nucleotide sequence 5′- AGAGGAGAGCCGUGUAUGAC -3′ (SEQ ID NO: 28) or 5′-AGAGGAGAGCCGTGTATGAC-3′ (SEQ ID NO: 77) with one substitution or two substitutions. In certain aspects, the miRNA inhibitor comprises the nucleotide sequence 5′-AGAGGAGAGCCGUGUAUGAC-3′ (SEQ ID NO: 28) or 5′-AGAGGAGAGCCGTGTATGAC-3′ (SEQ ID NO: 77). In certain aspects, the miRNA inhibitor comprises the nucleotide sequence 5′-AGAGGAGAGCCGUGUAUGAC-3′ (SEQ ID NO: 28).

In some aspects, a miR-485 inhibitor of the present disclosure comprises the sequence disclosed herein, e.g., any one of SEQ ID NOs: 2 to 30, and at least one, at least two, at least three, at least four or at least five additional nucleic acids at the N terminus, at least one, at least two, at least three, at least four, or at least five additional nucleic acids at the C terminus, or both. In some aspects, a miR-485 inhibitor of the present disclosure comprises the sequence disclosed herein, e.g., any one of SEQ ID NOs: 2 to 30, and one additional nucleic acid at the N terminus and/or one additional nucleic acid at the C terminus. In some aspects, a miR-485 inhibitor of the present disclosure comprises the sequence disclosed herein, e.g., any one of SEQ ID NOs: 2 to 30, and one or two additional nucleic acids at the N terminus and/or one or two additional nucleic acids at the C terminus. In some aspects, a miR-485 inhibitor of the present disclosure comprises the sequence disclosed herein, e.g., any one of SEQ ID NOs: 2 to 30, and one to three additional nucleic acids at the N terminus and/or one to three additional nucleic acids at the C terminus. In some aspects, a miR-485 inhibitor comprises 5′-GAGAGGAGAGCCGUGUAUGAC-3′ (SEQ ID NO: 29).

In some aspects, a miR-485 inhibitor of the present disclosure comprises one miR-485 binding site. In further aspects, a miR-485 inhibitor disclosed herein comprises at least two miR-485 binding sites. In certain aspects, a miR-485 inhibitor comprises three miR-485 binding sites. In some aspects, a miR-485 inhibitor comprises four miR-485 binding sites. In some aspects, a miR-485 inhibitor comprises five miR-485 binding sites. In certain aspects, a miR-485 inhibitor comprises six or more miR-485 binding sites. In some aspects, all the miR-485 binding sites are identical. In some aspects, all the miR-485 binding sites are different. In some aspects, at least one of the miR-485 binding sites is different. In some aspects, all the miR-485 binding sites are miR-485-3p binding sites. In other aspects, all the miR-485 binding sites are miR-485-5p binding sites. In further aspects, a miR-485 inhibitor comprises at least one miR-485-3p binding site and at least one miR-485-5p binding site.

### III.a. Chemically Modified Polynucleotides

In some aspects, a miR-485 inhibitor disclosed herein comprises a polynucleotide which includes at least one chemically modified nucleoside and/or nucleotide. When the polynucleotides of the present disclosure are chemically modified the polynucleotides can be referred to as “modified polynucleotides.”

A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside including a phosphate group. Modified nucleotides can be synthesized by any useful method, such as, for example, chemically, enzymatically, or recombinantly, to include one or more modified or non-natural nucleosides.

Polynucleotides can comprise a region or regions of linked nucleosides. Such regions can have variable backbone linkages. The linkages can be standard phosphodiester linkages, in which case the polynucleotides would comprise regions of nucleotides.

The modified polynucleotides disclosed herein can comprise various distinct modifications. In some aspects, the modified polynucleotides contain one, two, or more (optionally different) nucleoside or nucleotide modifications. In some aspects, a modified polynucleotide can exhibit one or more desirable properties, e.g., improved thermal or chemical stability, reduced immunogenicity, reduced degradation, increased binding to the target microRNA, reduced non-specific binding to other microRNA or other molecules, as compared to an unmodified polynucleotide.

In some aspects, a polynucleotide of the present disclosure (e.g., a miR-485 inhibitor) is chemically modified. As used herein, in reference to a polynucleotide, the terms “chemical modification” or, as appropriate, “chemically modified” refer to modification with respect to adenosine (A), guanosine (G), uridine (U), thymidine (T) or cytidine (C) ribo- or deoxyribonucleosides in one or more of their position, pattern, percent or population, including, but not limited to, its nucleobase, sugar, backbone, or any combination thereof.

In some aspects, a polynucleotide of the present disclosure (e.g., a miR-485 inhibitor) can have a uniform chemical modification of all or any of the same nucleoside type or a population of modifications produced by downward titration of the same starting modification in all or any of the same nucleoside type, or a measured percent of a chemical modification of all any of the same nucleoside type but with random incorporation In further aspects, the polynucleotide of the present disclosure (e.g., a miR-485 inhibitor) can have a uniform chemical modification of two, three, or four of the same nucleoside type throughout the entire polynucleotide (such as all uridines and/or all cytidines, etc. are modified in the same way).

Modified nucleotide base pairing encompasses not only the standard adenine-thymine, adenine-uracil, or guanine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleobase inosine and adenine, cytosine or uracil. Any combination of base/sugar or linker can be incorporated into polynucleotides of the present disclosure.

The skilled artisan will appreciate that, except where otherwise noted, polynucleotide sequences set forth in the instant application will recite “T”s in a representative DNA sequence but where the sequence represents RNA, the “T”s would be substituted for “U”s. For example, TD's of the present disclosure can be administered as RNAs, as DNAs, or as hybrid molecules comprising both RNA and DNA units.

In some aspects, the polynucleotide (e.g., a miR-485 inhibitor) includes a combination of at least two (e.g., 2, 3, 4, 5, 6, 7, 8, 8, 10, 11, 12, 13, 14, 15, 16, 17, 18, 18, 20 or more) modified nucleobases.

In some aspects, the nucleobases, sugar, backbone linkages, or any combination thereof in a polynucleotide are modified by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100%.

### (i) Base Modification

In certain aspects, the chemical modification is at nucleobases in a polynucleotide of the present disclosure (e.g., a miR-485 inhibitor). In some aspects, the at least one chemically modified nucleoside is a modified uridine (e.g., pseudouridine (ψ), 2-thiouridine (s2U), 1-methyl-pseudouridine (m1ψ), 1-ethyl-pseudouridine (e1ψ), or 5-methoxy-uridine (mo5U)), a modified cytosine (e.g., 5-methyl-cytidine (m5C)) a modified adenosine (e.g, 1-methyl-adenosine (m1A), N6-methyl-adenosine (m6A), or 2-methyl-adenine (m2A)), a modified guanosine (e.g., 7-methyl-guanosine (m7G) or 1-methyl-guanosine (m1G)), or a combination thereof.

In some aspects, the polynucleotide of the present disclosure (e.g., a miR-485 inhibitor) is uniformly modified (e.g., fully modified, modified throughout the entire sequence) for a particular modification. For example, a polynucleotide can be uniformly modified with the same type of base modification, e.g., 5-methyl-cytidine (m5C), meaning that all cytosine residues in the polynucleotide sequence are replaced with 5-methyl-cytidine (m5C). Similarly, a polynucleotide can be uniformly modified for any type of nucleoside residue present in the sequence by replacement with a modified nucleoside such as any of those set forth above.

In some aspects, the polynucleotide of the present disclosure (e.g., a miR-485 inhibitor) includes a combination of at least two (e.g., 2, 3, 4 or more) of modified nucleobases. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% of a type of nucleobases in a polynucleotide of the present disclosure (e.g., a miR-485 inhibitor) are modified nucleobases.

### (ii) Backbone Modifications

In some aspects, the polynucleotide of the present disclosure (i.e., miR-485 inhibitor) can include any useful linkage between the nucleosides. Such linkages, including backbone modifications, that are useful in the composition of the present disclosure include, but are not limited to the following: 3′-alkylene phosphonates, 3′-amino phosphoramidate, alkene containing backbones, aminoalkylphosphoramidates, aminoalkylphosphotriesters, boranophosphates, —CH₂—O—N(CH₃)—CH₂—, —CH₂—N(CH₃)—N(CH₃)—CH₂—, —CH₂—NH—CH₂—, chiral phosphonates, chiral phosphorothioates, formacetyl and thioformacetyl backbones, methylene (methylimino), methylene formacetyl and thioformacetyl backbones, methyleneimino and methylenehydrazino backbones, morpholino linkages, —N(CH₃)—CH₂—CH₂—, oligonucleosides with heteroatom internucleoside linkage, phosphinates, phosphoramidates, phosphorodithioates, phosphorothioate internucleoside linkages, phosphorothioates, phosphotriesters, PNA, siloxane backbones, sulfamate backbones, sulfide sulfoxide and sulfone backbones, sulfonate and sulfonamide backbones, thionoalkylphosphonates, thionoalkylphosphotriesters, and thionophosphoramidates.

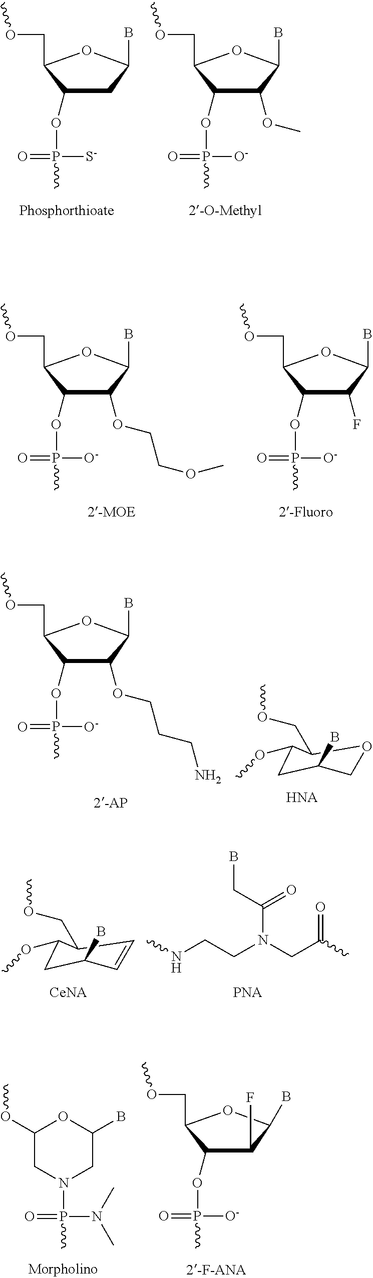

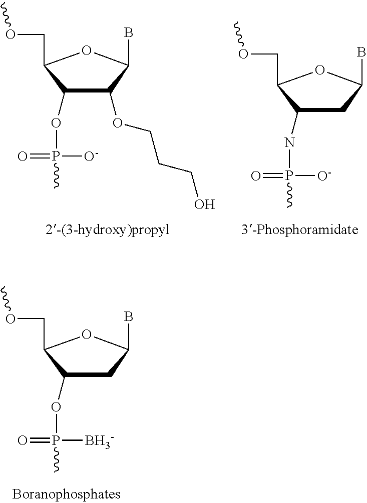

In some aspects, the presence of a backbone linkage disclosed above increase the stability and resistance to degradation of a polynucleotide of the present disclosure (i.e., miR-485 inhibitor).

In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% of the backbone linkages in a polynucleotide of the present disclosure (i.e., miR-485 inhibitor) are modified (e.g., all of them are phosphorothioate).

In some aspects, a backbone modification that can be included in a polynucleotide of the present disclosure (i.e., miR-485 inhibitor) comprises phosphorodiamidate morpholino oligomer (PMO) and/or phosphorothioate (PS) modification.

### (iii) Sugar Modifications

The modified nucleosides and nucleotides which can be incorporated into a polynucleotide of the present disclosure (i.e., miR-485 inhibitor) can be modified on the sugar of the nucleic acid. In some aspects, the sugar modification increases the affinity of the binding of a miR-485 inhibitor to a miR-485 nucleic acid sequence. Incorporating affinity-enhancing nucleotide analogues in the miR-485 inhibitor, such as LNA or 2′-substituted sugars, can allow the length and/or the size of the miR-485 inhibitor to be reduced.

In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or about 100% of the nucleotides in a polynucleotide of the present disclosure (i.e., miR-485 inhibitor) contain sugar modifications (e.g., LNA).

In some aspects, at least 1, 2, 3, 4, 5, 6, 7, 8, 9, 10, 11, 12, 13, 14, 15, 16, 17, 18, 19, 20, 21, or 22 nucleotide units in a polynucleotide of the present disclosure are sugar modified (e.g., LNA).

Generally, RNA includes the sugar group ribose, which is a 5-membered ring having an oxygen. Exemplary, non-limiting modified nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2′)) , and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar.

The 2′ hydroxyl group (OH) of ribose can be modified or replaced with a number of different substituents. Exemplary substitutions at the 2′-position include, but are not limited to, H, halo, optionally substituted C₁₋₆ alkyl; optionally substituted C₁₋₆ alkoxy; optionally substituted C₆₋₁₀ aryloxy; optionally substituted C₃₋₈ cycloalkyl; optionally substituted C₃₋₈ cycloalkoxy; optionally substituted C₆₋₁₀ aryloxy; optionally substituted C₆₋₁₀ aryl-C₁₋₆ alkoxy, optionally substituted C₁₋₁₂ (heterocyclyl)oxy; a sugar (e.g., ribose, pentose, or any described herein); a polyethyleneglycol (PEG), —O(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C₁₋₆ alkylene or C₁₋₆ heteroalkylene bridge to the 4′-carbon of the same ribose sugar, where exemplary bridges include methylene, propylene, ether, amino bridges, aminoalkyl, aminoalkoxy, amino, and amino acid.

In some aspects, nucleotide analogues present in a polynucleotide of the present disclosure (i.e., mir-485 inhibitor) comprise, e.g., 2′-O-alkyl-RNA units, 2′-OMe-RNA units, 2′-O-alkyl-SNA, 2′-amino-DNA units, 2′-fluoro-DNA units, LNA units, arabino nucleic acid (ANA) units, 2′-fluoro-ANA units, HNA units, INA (intercalating nucleic acid) units, 2′MOE units, or any combination thereof. In some aspects, the LNA is, e.g., oxy-LNA (such as beta-D-oxy-LNA, or alpha-L-oxy-LNA), amino-LNA (such as beta-D-amino-LNA or alpha-L-amino-LNA), thio-LNA (such as beta-D-thio0-LNA or alpha-L-thio-LNA), ENA (such a beta-D-ENA or alpha-L-ENA), or any combination thereof. In further aspects, nucleotide analogues that can be included in a polynucleotide of the present disclosure (i.e., miR-485 inhibitor) comprises a locked nucleic acid (LNA), an unlocked nucleic acid (UNA), an arabino nucleic acid (ABA), a bridged nucleic acid (BNA), and/or a peptide nucleic acid (PNA).

In some aspects, a polynucleotide of the present disclosure (i.e., miR-485 inhibitor) can comprise both modified RNA nucleotide analogues (e.g., LNA) and DNA units. In some aspects, a miR-485 inhibitor is a gapmer. See, e.g., U.S. Pat. Nos. 8,404,649; 8,580,756; 8,163,708; 9,034,837; all of which are herein incorporated by reference in their entireties. In some aspects, a miR-485 inhibitor is a micromir. See U.S. Pat. Appl. Publ. No. US20180201928, which is herein incorporated by reference in its entirety.

In some aspects, a polynucleotide of the present disclosure (i.e., miR-485 inhibitor) can include modifications to prevent rapid degradation by endo- and exo-nucleases. Modifications include, but are not limited to, for example, (a) end modifications, e.g., 5′ end modifications (phosphorylation, dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages.

### IV. Vectors and Delivery Systems

In some aspects, the miR-485 inhibitors of the present disclosure can be administered, e.g., to a subject at risk of hair loss, using any relevant delivery system known in the art. In certain aspects, the delivery system is a vector. Accordingly, in some aspects, the present disclosure provides a vector comprising a miR-485 inhibitor of the present disclosure.

In some aspects, the vector is a viral vector. In some aspects, the viral vector is an adenoviral vector or an adenoassociated viral vector. In certain aspects, the viral vector is an AAV that has a serotype of AAV2, AAV3, AAV4, AAV5, AAV6, AAV7, AAV8, AAV9, AAV10, or any combination thereof. In some aspects, the adenoviral vector is a third generation adenoviral vector. ADEASY™ is by far the most popular method for creating adenoviral vector constructs. The system consists of two types of plasmids: shuttle (or transfer) vectors and adenoviral vectors. The transgene of interest is cloned into the shuttle vector, verified, and linearized with the restriction enzyme PmeI. This construct is then transformed into ADEASIER-1 cells, which are BJ5183 E. coli cells containing PADEASY™. PADEASY™ is a ˜33 Kb adenoviral plasmid containing the adenoviral genes necessary for virus production. The shuttle vector and the adenoviral plasmid have matching left and right homology arms which facilitate homologous recombination of the transgene into the adenoviral plasmid. One can also co-transform standard BJ5183 with supercoiled PADEASY™ and the shuttle vector, but this method results in a higher background of non-recombinant adenoviral plasmids. Recombinant adenoviral plasmids are then verified for size and proper restriction digest patterns to determine that the transgene has been inserted into the adenoviral plasmid, and that other patterns of recombination have not occurred. Once verified, the recombinant plasmid is linearized with PacI to create a linear dsDNA construct flanked by ITRs. 293 or 911 cells are transfected with the linearized construct, and virus can be harvested about 7-10 days later. In addition to this method, other methods for creating adenoviral vector constructs known in the art at the time the present application was filed can be used to practice the methods disclosed herein.

In some aspects, the viral vector is a retroviral vector, e.g., a lentiviral vector (e.g., a third or fourth generation lentiviral vector). Lentiviral vectors are usually created in a transient transfection system in which a cell line is transfected with three separate plasmid expression systems. These include the transfer vector plasmid (portions of the HIV provirus), the packaging plasmid or construct, and a plasmid with the heterologous envelop gene (env) of a different virus. The three plasmid components of the vector are put into a packaging cell which is then inserted into the HIV shell. The virus portions of the vector contain insert sequences so that the virus cannot replicate inside the cell system. Current third generation lentiviral vectors encode only three of the nine HIV-1 proteins (Gag, Pol, Rev), which are expressed from separate plasmids to avoid recombination-mediated generation of a replication-competent virus. In fourth generation lentiviral vectors, the retroviral genome has been further reduced (see, e.g., TAKARA® LENTI-X™ fourth-generation packaging systems).

Any AAV vector known in the art can be used in the methods disclosed herein. The AAV vector can comprise a known vector or can comprise a variant, fragment, or fusion thereof. In some aspects, the AAV vector is selected from the group consisting of AAV type 1 (AAV1), AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, bovine AAV, shrimp AVV, snake AVV, and any combination thereof.

In some aspects, the AAV vector is derived from an AAV vector selected from the group consisting of AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, and any combination thereof.

In some aspects, the AAV vector is a chimeric vector derived from at least two AAV vectors selected from the group consisting of AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, and any combination thereof.

In certain aspects, the AAV vector comprises regions of at least two different AAV vectors known in the art.

In some aspects, the AAV vector comprises an inverted terminal repeat from a first AAV (e.g., AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, or any derivative thereof) and a second inverted terminal repeat from a second AAV (e.g., AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, or any derivative thereof).

In some aspects, the AVV vector comprises a portion of an AAV vector selected from the group consisting of AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, and any combination thereof. In some aspects, the AAV vector comprises AAV2.

In some aspects, the AVV vector comprises a splice acceptor site. In some aspects, the AVV vector comprises a promoter. Any promoter known in the art can be used in the AAV vector of the present disclosure. In some aspects, the promoter is an RNA Pol III promoter. In some aspects, the RNA Pol III promoter is selected from the group consisting of the U6 promoter, the H1 promoter, the 7SK promoter, the 5S promoter, the adenovirus 2 (Ad2) VAI promoter, and any combination thereof. In some aspects, the promoter is a cytomegalovirus immediate-early gene (CMV) promoter, an EF1a promoter, an SV40 promoter, a PGK1 promoter, a Ubc promoter, a human beta actin promoter, a CAG promoter, a TRE promoter, a UAS promoter, a Ac5 promoter, a polyhedrin promoter, a CaMKIIa promoter, a GAL1 promoter, a GAL10 promoter, a TEF promoter, a GDS promoter, a ADH1 promoter, a CaMV35S promoter, or a Ubi promoter. In a specific aspect, the promoter comprises the U6 promoter.

In some aspects, the AAV vector comprises a constitutively active promoter (constitutive promoter). In some aspects, the constitutive promoter is selected from the group consisting of hypoxanthine phosphoribosyl transferase (HPRT), adenosine deaminase, pyruvate kinase, beta-actin promoter, cytomegalovirus (CMV), simian virus (e.g., SV40), papilloma virus, adenovirus, human immunodeficiency virus (HIV), Rous sarcoma virus, a retrovirus long terminal repeat (LTR), Murine stem cell virus (MSCV) and the thymidine kinase promoter of herpes simplex virus.

In some aspects, the promoter is an inducible promoter. In some aspects, the inducible promoter is a tissue specific promoter. In certain aspects, the tissue specific promoter drives transcription of the coding region of the AVV vector in a neuron, a glial cell, or in both a neuron and a glial cell.

In some aspects, the AVV vector comprises one or more enhancers. In some aspects, the one or more enhancer is present in the AAV alone or together with a promoter disclosed herein. In some aspects, the AAV vector comprises a 3′UTR poly(A) tail sequence. In some aspects, the 3′UTR poly(A) tail sequence is selected from the group consisting of bGH poly(A), actin poly(A), hemoglobin poly(A), and any combination thereof. In some aspects, the 3′UTR poly(A) tail sequence comprises bGH poly(A).

In some aspects, a miR-485 inhibitor disclosed herein is administered with a delivery agent. Non-limiting examples of delivery agents that can be used include an exosome, a lipidoid, a liposome, a lipoplex, a lipid nanoparticle, an extracellular vesicle, a polymeric compound, a peptide, a protein, a cell, a nanoparticle mimic, a nanotube, a micelle, a viral vector, or a conjugate.

Thus, in some aspects, the present disclosure also provides a composition comprising a miRNA inhibitor of the present disclosure (i.e., miR-485 inhibitor) and a delivery agent. In some aspects, the delivery agent comprises a earlier unit, e.g., that can self-assemble into micelles or be incorporated into micelles. In some aspects, the delivery agent comprises a cationic carrier unit comprising

[WP]-L1-[CC]-L2-[AM] (formula I)

or

[WP]-L1-[AM]-L2-[CC] (formula II)
wherein
WP is a water-soluble biopolymer moiety;
CC is a positively charged (i.e., cationic) carrier moiety;
AM is an adjuvant moiety; and,
L1 and L2 are independently optional linkers, and
wherein when mixed with a nucleic acid at an ionic ratio of about 1:1, the cationic carrier unit forms a micelle. Accordingly, in some aspects, the miRNA inhibitor and the cationic carrier unit are capable of associating with each other (e.g., via a covalent bond or a non-valent bond) to form a micelle when mixed together.

In some aspects, composition comprising a miRNA inhibitor of the present disclosure (i.e., miR-485 inhibitor) interacts with the cationic carrier unit via an ionic bond.

In some aspects, the water-soluble polymer comprises poly(alkylene glycols), poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyglycerol, polyphosphazene, polyoxazolines (“POZ”) poly(N-acryloylmorpholine), or any combinations thereof. In some aspects, the water-soluble polymer comprises polyethylene glycol (“PEG”), polyglycerol, or poly(propylene glycol) (“PPG”). In some aspects, the water-soluble polymer comprises:

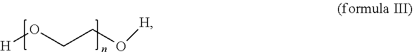

wherein n is 1-1000.

In some aspects, then is at least about 110, at least about 111, at least about 112, at least about 113, at least about 114, at least about 115, at least about 116, at least about 117, at least about 118, at least about 119, at least about 120, at least about 121, at least about 122, at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, or at least about 141. In some aspects, the n is about 80 to about 90, about 90 to about 100, about 100 to about 110, about 110 to about 120, about 120 to about 130, about 140 to about 150, about 150 to about 160.

In some aspects, the water-soluble polymer is linear, branched, or dendritic. In some aspects, the cationic carrier moiety comprises one or more basic amino acids. In some aspects, the cationic carrier moiety comprises at least three, at least four, at least five, at least six, at least seven, at least eight, at least nine, at least ten, at least 11, at least 12, at least 13, at least 14, at last 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 basic amino acids. In some aspects, the cationic carrier moiety comprises about 30 to about 50 basic amino acids. In some aspects, the basic amino acid comprises arginine, lysine, histidine, or any combination thereof. In some aspects, the cationic carrier moiety comprises about 40 lysine monomers.

In some aspects, the adjuvant moiety is capable of modulating an immune response, an inflammatory response, and/or a tissue microenvironment. In some aspects, the adjuvant moiety comprises an imidazole derivative, an amino acid, a vitamin, or any combination thereof. In some aspects, the adjuvant moiety comprises:

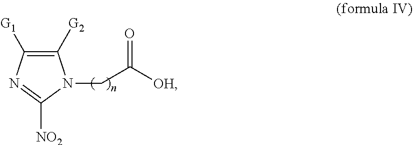

wherein each of G1 and G2 is H, an aromatic ring, or 1-10 alkyl, or G1 and G2 together form an aromatic ring, and wherein n is 1-10.

In some aspects, the adjuvant moiety comprises nitroimidazole. In some aspects, the adjuvant moiety comprises metronidazole, tinidazole, nimorazole, dimetridazole, pretomanid, ornidazole, megazol, azanidazole, benznidazole, or any combination thereof. In some aspects, the adjuvant moiety comprises an amino acid.

In some aspects, the adjuvant moiety comprises

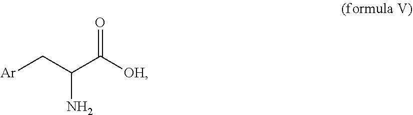

wherein Ar is

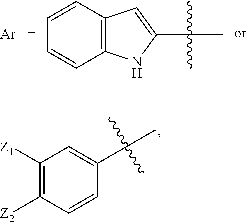

and wherein each of Z1 and Z2 is H or OH.

In some aspects, the adjuvant moiety comprises a vitamin. In some aspects, the vitamin comprises a cyclic ring or cyclic hetero atom ring and a carboxyl group or hydroxyl group. In some aspects, the vitamin comprises:

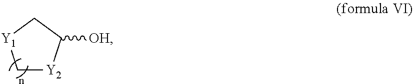

wherein each of Y1 and Y2 is C, N, O, or S, and wherein n is 1 or 2.

In some aspects, the vitamin is selected from the group consisting of vitamin A, vitamin B1, vitamin B2, vitamin B3, vitamin B6, vitamin B7, vitamin B9, vitamin B12, vitamin C, vitamin D2, vitamin D3, vitamin E, vitamin M, vitamin H, and any combination thereof. In some aspects, the vitamin is vitamin B3.

In some aspects, the adjuvant moiety comprises at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, or at least about 20 vitamin B3. In some aspects, the adjuvant moiety comprises about 10 vitamin B3.

In some aspects, the composition comprises a water-soluble biopolymer moiety with about 120 to about 130 PEG units, a cationic carrier moiety comprising a poly-lysine with about 30 to about 40 lysines, and an adjuvant moiety with about 5 to about 10 vitamin B3.

In some aspects, the composition comprises (i) a water-soluble biopolymer moiety with about 100 to about 200 PEG units, (ii) about 30 to about 40 lysines with an amine group (e.g., about 32 lysines), (iii) about 15 to 20 lysines, each having a thiol group (e.g., about 16 lysines, each with a thiol group), and (iv) about 30 to 40 lysines fused to vitamin B3 (e.g., about 32 lysines, each fused to vitamin B3). In some aspects, the composition further comprises a targeting moiety, e.g., a LAT1 targeting ligand, e.g., phenyl alanine, linked to the water soluble polymer. In some aspects, the thiol groups in the composition form disulfide bonds.

In some aspects, the composition comprises (1) a micelle comprising (i) about 100 to about 200 PEG units, (ii) about 30 to about 40 lysines with an amine group (e.g., about 32 lysines), (iii) about 15 to 20 lysines, each having a thiol group (e.g., about 16 lysines, each with a thiol group), and (iv) about 30 to 40 lysines fused to vitamin B3 (e.g., about 32 lysines, each fused to vitamin B3), and (2) a miR485 inhibitor (e.g., SEQ ID NO: 28), wherein the miR485 inhibitor is encapsulated within the micelle. In some aspects, the composition further comprises a targeting moiety, e.g., a LAT1 targeting ligand, e.g., phenyl alanine, linked to the PEG units. In some aspects, the thiol groups in the micelle form disulfide bonds.

The present disclosure also provides a micelle comprising a miRNA inhibitor of the present disclosure (i.e., miR-485 inhibitor) wherein the miRNA inhibitor and the delivery agent are associated with each other.

In some aspects, the association is a covalent bond, a non-covalent bond, or an ionic bond. In some aspects, the positive charge of the cationic carrier moiety of the cationic carrier unit is sufficient to form a micelle when mixed with the miR-485 inhibitor disclosed herein in a solution, wherein the overall ionic ratio of the positive charges of the cationic carrier moiety of the cationic carrier unit and the negative charges of the miR-485 inhibitor (or vector comprising the inhibitor) in the solution is about 1:1.

In some aspects, the cationic carrier unit is capable of protecting the miRNA inhibitor of the present disclosure (i.e., miR-485 inhibitor) from enzymatic degradation. See PCT Publication No. WO2020/261227, which is herein incorporated by reference in its entirety.

### V. Pharmaceutical Compositions

In some aspects, the present disclosure also provides pharmaceutical compositions comprising a miR-485 inhibitor disclosed herein (e.g., a polynucleotide or a vector comprising the miR-485 inhibitor) that are suitable for administration to a subject. The pharmaceutical compositions generally comprise a miR-485 inhibitor described herein (e.g., a polynucleotide or a vector) and a pharmaceutically-acceptable excipient or carrier in a form suitable for administration to a subject. Pharmaceutically acceptable excipients or carriers are determined in part by the particular composition being administered, as well as by the particular method used to administer the composition.

Accordingly, there is a wide variety of suitable formulations of pharmaceutical compositions comprising a miR-485 inhibitor of the present disclosure. (See, e.g., Remington's Pharmaceutical Sciences, Mack Publishing Co., Easton, Pa. 18th ed. (1990)). The pharmaceutical compositions are generally formulated sterile and in full compliance with all Good Manufacturing Practice (GMP) regulations of the U.S. Food and Drug Administration.

### VI. Kits

The present disclosure also provides kits or products of manufacture, comprising a miRNA inhibitor of the present disclosure (e.g., a polynucleotide, vector, or pharmaceutical composition disclosed herein) and optionally instructions for use, e.g., instructions for use according to the methods disclosed herein. In some aspects, the kit or product of manufacture comprises a miR-485 inhibitor (e.g., vector, e.g., an AAV vector, a polynucleotide, or a pharmaceutical composition of the present disclosure) in one or more containers. In some aspects, the kit or product of manufacture comprises miR-485 inhibitor (e.g., a vector, e.g., an AAV vector, a polynucleotide, or a pharmaceutical composition of the present disclosure) and a brochure. One skilled in the art will readily recognize that miR-485 inhibitors disclosed herein (e.g., vectors, polynucleotides, and pharmaceutical compositions of the present disclosure, or combinations thereof) can be readily incorporated into one of the established kit formats which are well known in the art.

The following examples are offered by way of illustration and not by way of limitation.

### EXAMPLES

### Example 1: Preparation of miR-485 Inhibitor

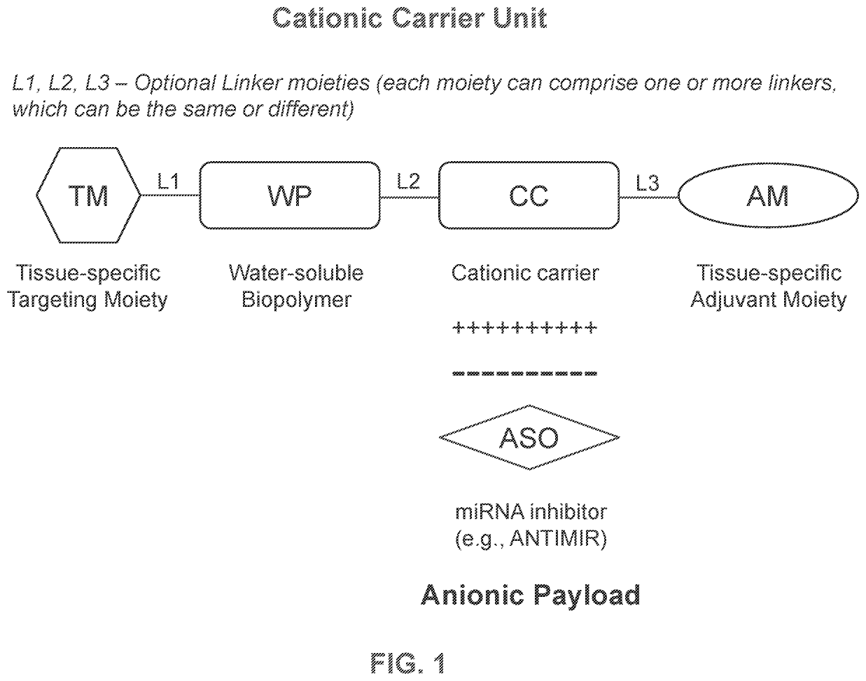

(a) Synthesis of alkyne modified tyrosine: An alkyne modified tyrosine was generated as an intermediate for the synthesis of a tissue specific targeting moiety (TM, see FIG. 1) of a cationic carrier unit to direct micelles of the present disclosure to the LAT1 transporter in the BBB.

A mixture of N-(tert-butoxycarbonyl)-L-tyrosine methyl ester (Boc-Tyr-OMe) (0.5 g, 1.69 mmol) and K₂CO₃ (1.5 equiv., 2.54 mmol) in acetonitrile (4.0 ml) was added drop by drop to propargyl bromide (1.2 equiv., 2.03 mmol). The reaction mixture was heated at 60° C. overnight. After the reaction, the reaction mixture was extracted using water:ethyl acetate (EA). Then, the organic layer was washed using a brine solution. The crude material was purified by flash column (EA in hexane 10%). Next, the resulting product was dissolved in 1,4-dioxane (1.0 ml) and 6.0 M HCl (1.0 ml). The reaction mixture was heated at 100° C. overnight. Next, the dioxane was removed and extracted by EA. Aqueous NaOH (0.5 M) solution was added to the mixture until the pH value become 7. The reactant was concentrated by evaporator and centrifuged at 12,000 rpm at 0° C. The precipitate was washed with deionized water and lyophilized.

(b) Synthesis of poly(ethylene glycol)-b-poly(L-lysine) (PEG-PLL): This synthesis step generated the water-soluble biopolymer (WP) and cationic carrier (CC) of a cationic carrier unit of the present disclosure (see FIG. 1).

Poly(ethylene glycol)-b-poly(L-lysine) was synthesized by ring opening polymerization of Lys(TFA)-NCA with monomethoxy PEG (MeO-PEG) as a macroinitiator. In brief, MeO-PEG (600 mg, 0.12 mmol) and Lys(TFA)-NCA (2574 mg, 9.6 mmol) were separately dissolved in DMF containing 1M thiourea and DMF(or NMP). Lys(TFA)-NCA solution was dropped into the MeO-PEG solution by micro syringe and the reaction mixture was stirred at 37° C. for 4 days. The reaction bottles were purged with argon and vacuum. All reactions were conducted in argon atmosphere. After the reaction, the mixture was precipitated into an excess amount of diethyl ether. The precipitate was re-dissolved in methanol and precipitated again into cold diethyl ether. Then it was filtered and white powder was obtained after drying in vacuo. For the deprotection of TFA group in PEG-PLL(TFA), the next step was followed.

MeO-PEG-PLL(TFA) (500 mg) was dissolved in methanol (60 mL) and 1N NaOH (6 mL) was dropped into the polymer solution with stirring. The mixture was maintained for 1 day with stirring at 37° C. The reaction mixture was dialyzed against 10 mM HEPES for 4 times and distilled water. White powder of PEG-PLL was obtained after lyophilization.

(b) Synthesis of azido-poly(ethylene glycol)-b-poly(L-lysine) (N₃-PEG-PLL): This synthesis step generated the water-soluble biopolymer (WP) and cationic carrier (CC) of a cationic carrier unit of the present disclosure (see FIG. 1).

Azido-poly(ethylene glycol)-b-poly(L-lysine) was synthesized by ring opening polymerization of Lys(TFA)-NCA with azido-PEG (N₃-PEG). In brief, N₃-PEG (300 mg, 0.06 mmol) and Lys(TFA)-NCA (1287 mg, 4.8 mmol) were separately dissolved in DMF containing 1M thiourea and DMF(or NMP). Lys(TFA)-NCA solution was dropped into the N₃-PEG solution by micro syringe and the reaction mixture was stirred at 37° C. for 4 days. The reaction bottles were purged with argon and vacuum. All reactions were conducted in argon atmosphere. After the reaction, the mixture was precipitated into an excess amount of diethyl ether. The precipitate was re-dissolved in methanol and precipitated again into cold diethyl ether. Then it was filtered and white powder was obtained after drying in vacuo. For the deprotection of TFA group in PEG-PLL(TFA), the next step was followed.

N₃-PEG-PLL (500 mg) was dissolved in methanol (60 mL) and 1N NaOH (6 mL) was dropped into the polymer solution with stirring. The mixture was maintained for 1 day with stirring at 37° C. The reaction mixture was dialyzed against 10 mM HEPES for 4 times and distilled water. White powder of N₃-PEG-PLL was obtained after lyophilization.

(c) Synthesis of (methoxy or) azido-poly(ethylene glycol)-b-poly(L-lysine/nicotinamide/mercaptopropanamide) (N₃-PEG-PLL(Nic/SH)): In this step, the tissue-specific adjuvant moieties (AM, see FIG. 1) were attached to the WP-CC component of a cationic carrier unit of the present disclosure. The tissue-specific adjuvant moiety (AM) used in the cationic carrier unit was nicotinamide (vitamin B3). This step would yield the WP-CC-AM components of the cationic carrier unit depicted in FIG. 1.

Azido-poly(ethylene glycol)-b-poly(L-lysine/nicotinamide/mercaptopropanamide) (N₃-PEG-PLL(Nic/SH)) was synthesized by chemical modification of N₃-PEG-PLL and nicotinic acid in the presence of EDC/NHS. N₃-PEG-PLL (372 mg, 25.8 μmol) and nicotinic acid (556.7 mg, 1.02 equiv. to NH2 of PEG-PLL) were separately dissolved in mixture of deionized water and methanol (1:1). EDC•HCl (556.7 mg, 1.5 equiv. to NH₂ of N₃-PEG-PLL) was added into nicotinic acid solution and NHS (334.2 mg, 1.5 equiv. to NH2 of PEG-PLL) stepwise added into the mixture.

The reaction mixture was added into the N₃-PEG-PLL solution. The reaction mixture was maintained at 37° C. for 16 hours with stirring. After 16 hours, 3,3′-dithiodiproponic acid (36.8 mg, 0.1 equiv.) was dissolved in methanol, EDC•HCl (40.3 mg, 0.15 equiv.), and NHS (24.2 mg, 0.15 equiv.) were dissolved each in deionized water. Then, NHS and EDC•HCl were added sequentially into 3,3′-dithiodiproponic acid solution. The mixture solution was stirred for 4 hours at 37° C. after adding crude N₃-PEG-PLL(Nic) solution.

For purification, the mixture was dialyzed against methanol for 2 hours, added DL-dithiothreitol (DTT, 40.6 mg, 0.15 equiv.), then activated for 30 min.

For removing the DTT, the mixture was dialyzed sequentially methanol, 50% methanol in deionized water, deionized water

(d) Synthesis of Phenyl alanine-poly(ethylene glycol)-b-poly(L-lysine/nicotinamide/mercaptopropanamide) (Phe-PEG-PLL(Nic/SH)): In this step, the tissue-specific targeting moiety (TM) was attached to the WP-CC-AM component synthesized in the previous step. The TM component (phenyl alanine) was generated by reaction of the intermediate generated in step (a) with the product of step (c).

To target brain endothelial tissue in blood vessels, as a LAT1 targeting amino acid, phenyl alanine was introduced by click reaction between N₃-PEG-PLL(Nic/SH) and alkyne modified tyrosine in the presence of copper catalyst. In brief, N₃-PEG-PLL(Nic/SH) (130 mg, 6.5 μmol) and alkyne modified phenyl alanine (5.7 mg, 4.0 equiv.) were dissolved in deionized water (or 50 mM sodium phosphate buffer). Then, CuSO4.H2O (0.4 mg, 25 mol %) and Tris(3-hydroxypropyltriazolylmethyl)amine (THPTA, 3.4 mg, 1.2 equiv.) were dissolved deionized water and added N₃-PEG-PLL(Nic/SH) solution. Then, sodium ascorbate (3.2 mg, 2.5 equiv.) were added into the mixture solution. The reaction mixture was maintained with stirring for 16 hours at room temperature. After the reaction, the mixture was transferred into dialysis membranes (MWCO=7,000) and dialyzed against deionized water for 1 day. The final product was obtained after lyophilization.

(e) Polyion Complex (PIC) micelle preparation—Once the cationic carrier units of the present disclosure were generated as described above, micelles were produced. The micelles described in the present example comprised cationic carrier units combined with an antisense oligonucleotide payload.

Nano sized PIC micelles were prepared by mixing MeO- or Phe-PEG-PLL(Nic) and miRNA. PEG-PLL(Nic) was dissolved in HEPES buffer (10 mM) at 0.5 mg/mL concentration. Then a miRNA solution (22.5 μM) in RNAase free water was mixed with the polymer solution at 2:1 (v/v) ratio of miRNA inhibitor (SEQ ID NO: 28) (485 ASO-001) to polymer.

The mixing ratio of polymer to anti-miRNA was determined by optimizing micelle forming conditions, i.e., ratio between amine in polymer (carrier of the present disclosure) to phosphate in anti-miRNA (payload). The mixture of polymer (carrier) and anti-miRNA (payload) was vigorously mixed for 90 seconds by multi-vortex at 3000 rpm, and kept at room temperature for 30 min to stabilize the micelles.

Micelles (10 μM of Anti-miRNA concentration) were stored at 4° C. prior to use. MeO- or Phe- micelles were prepared using the same method, and different amounts of Phe-containing micelles (25%˜75%) were also prepared by mixing both polymers during micelle preparation.

### Example 2: Materials and Methods

Unless provided otherwise, the Examples described below use one or more of the following materials and methods:

### Assay for in Vivo Hair Growth in C57BL/6J Mice

C57BL/6J mice were obtained from DBL Co. Ltd. animal laboratory at 6 weeks

of age and allowed to adapt to their new environment for one week. To observe the synchronized telogen phase, hair was removed from each mouse by using electric clipper and were shaved dorsally by depilatory cream in telogen. Intramuscular (IM) injection of vehicle control (PBS), unless otherwise indicated or twice on (PD PD0 and PD7) Intramuscular (IM) injection of miR-485 inhibitor-485 ASO-001 (0.3 mpk and 0.6 mpk). Appearance of skin pigmentation and hair growth were monitored and documented by digital photomicrograph, with the experimenter(s) being blind to the treatment conditions. Progression was also assigned a value from 0 to 6 based on pigmentation levels and hair shaft density, with 0 indicating no hair growth (and no pigmentation) and higher number corresponding to darker skin and larger areas of dense hair growth. Scoring was done blindly. Images representing different scores are presented in hair regrowth quantification scale (0-6), where grade 0=skin pink, no pigmentation; grade 1=<30% skin area showing the darkening, but no visible hair; grade 2=30%-70% skin area showing the darkening, no visible hair; grade 3=>70% skin area showing the darkening, or 30% hair visible; grade 4=>70% skin area showing the darkening, and 30%-70% hair visible; grade 5=>70% skin area showing the darkening, and >70% hair visible; and grade 6=>90% skin area showing darkening, >90% and hair visible.

### Tissue Staining, Measurement of Histological Structure of Skin and Hair Follicle

The dorsal skin of all the groups was fixed in 4% PFA on PD10 and PD16 and assessed with hematoxylin and eosin (H&E) staining. The number of HFs were counted (cross-section) in dermis and subcutis. Thickness of dermis and subcutis was taken from the visible microscopic field (3 fields) with at-least 7 measurements. The length of hair follicle and diameter of hair bulb were measured from longitudinal and transverse section of dorsal skin by analyzing the images by Motic images plus 2.0 ML.

### Real Time PCR

Total RNA was isolated using the Isolation of small and large RNA kit (Macherey Nagel, Dren). The concentration of the collected RNA was measured with a nucleic acid quantification system (Nanodrop. Thermo Scientific Inc.). cDNA was synthesized using miScript II RT Kit (Qiagen, Hilden, Germany). For analysis the expression of miR-485-3p was performed by TaqMan miRNA analysis using TOPREAL™ qPCR 2× PreMIX (Enzynomics, Korea) on CFX connect system (Bio-Rad). The real-time PCR measurement of individual cDNAs was performed using Taq man probe to measure duplex DNA formation with the Bio-Rad real-time PCR system. Primers were as follows:

Probe:FAM-CGAGGTCGACTTCCTAGA-NFQ.miR-485 3p forward:5′CATACACGGCTCTCCTCTCTAAA-3′. The relative gene expression was analyzed by the 2^{-ΔΔC}_{T}.

### Western Blotting

Skin samples were harvested and on day 10 and 16 post depilation. Mouse skin tissue lysate was prepared by homogenization in ice-cold RIPA buffer (iNtRON Biotechnology) containing protease/phosphatase inhibitor cocktail (Cell Signaling Technology, Cat#5872). Tissue debris was removed by centrifugation at 13,000 rpm for 15 min at 4° C., and supernatants were collected. Lysate was boiled for 5 min in 1× SDS (Fisher Scientific) loading buffer containing 5% β-mercaptoethanol (Fisher Scientific). Samples were then subjected to SDS-PAGE on PAGE 8%, 12% Bis-Tris gels (Invitrogen) in MOPS SDS Running Buffer (Invitrogen) and transferred to PVDF membranes and incubated with the following primary antibodies: rabbit anti-CD36 (Abcam, Cat# ab80080, 1:1000), rabbit anti-VEGF-A (Abcam, Cat# ab51745, 1:1000), rabbit anti-Wnt3a (IBL,Cat#11088, 1:1000), rabbit anti-β-actin (Abcam, Cat#ab1997, 1:100), anti-actin (Santa Cruz, Cat#sc-47778). The results were visualized using an enhanced chemiluminescence system, and quantified by densitometric analysis (Image J software, NIH). All experiments were performed independently at least three times.

### Immunofluorescent Staining

C57BL/6J mice skin tissue was embedded in a frozen tissue-embedding agent (OTC Compound, Sakura Finetek Japan Co., Ltd.), and frozen section slides were prepared with a frozen section production system (Cryostat, Leica Camera AG). After fixing for 15 minutes with 4% PFA, the tissue was washed with PBS and allowed to react for 1 hour using a blocking solution obtained by adding 5% skim milk, 1% donkey serum and 0.1% TritonX-100 to PBS. Next, the tissue was allowed to react for 1 hour at room temperature or overnight at 4° C. using primary antibody solution obtained by diluting CD36 antibody solution (Invitrogen Cat# PA1-16813) 50-fold, with the blocking solution. After washing 3 time with PBS, the tissue was allowed to react for 1 hour at room temperature using a secondary antibody solution obtained by diluting FITC-labeled anti-mouse IgG antibody (Invitrogen Corp.) 200-fold each with blocking solution. After reacting with DAPI solution, the tissue was washed 3 times with PBS and sealed with an anti-fade reagent (Prolong Gold Antifade Reagent) and a cover glass. The tissue was observed using a fluorescence microscope (Olympus Corp.).

### Example 3. Analysis of Dose Dependent Hair Growth Promotion Effect of miR-485 Inhibitor

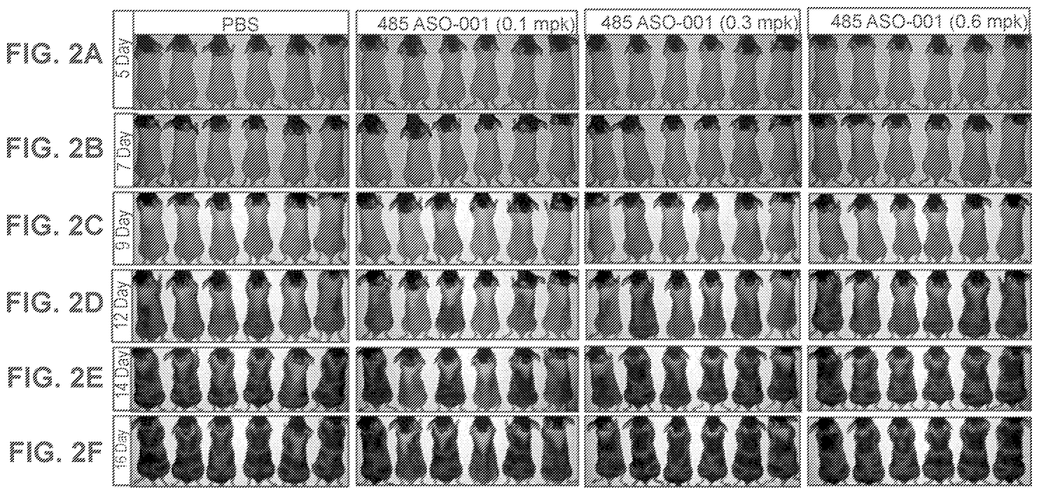

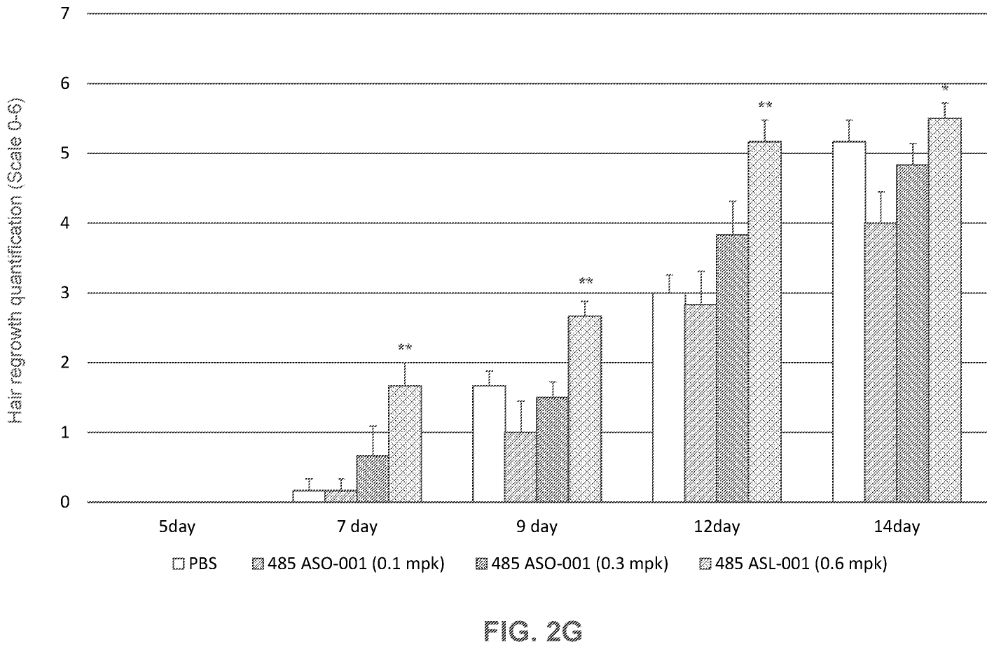

To begin evaluating the hair regrowth effect of the miR-485 inhibitor (485 ASO-001) (prepared as shown in Example 1) on depilated mice, six-week-old C57BL/6J mice were purchased and allowed to adapt to their new environment for one week. To observe the synchronized telogen phase, hair was removed from each mouse by using depilatory cream. Animals were then divided into five groups (n=6) to evaluate the hair regrowth effect of the miR-485 inhibitor (485 ASO-001). Negative control (PBS) and 485 ASO-001 (0.1 mpk, 0.3 mpk, and 0.6 mpk) were administered by a one-time intramuscular injection. The digital photographs of mice were taken post-depilation (pd.) on days 5 (FIG. 2A), 7 (FIG. 2B), 9 (FIG. 2C), 12 (FIG. 2D), 14 (FIG. 2E), and 16 (FIG. 2F) after treatment, and the hair growth area was analyzed via morphological observation by a score of 0-6 (FIG. 2G).

As shown in FIG. 2G, there was a significant increase observed in hair growth score on days 7 and 12 in 485 ASO-001 (0.3 mpk and 0.6 mpk) treated mice groups compared to the PBS-treated control group. Furthermore, 485 ASO-001 (0.6 mpk) treated mice group showed more hair regrowth compared to 485 ASO-001 (0.1 mpk and 0.3 mpk) treated mice groups (FIG. 2G).

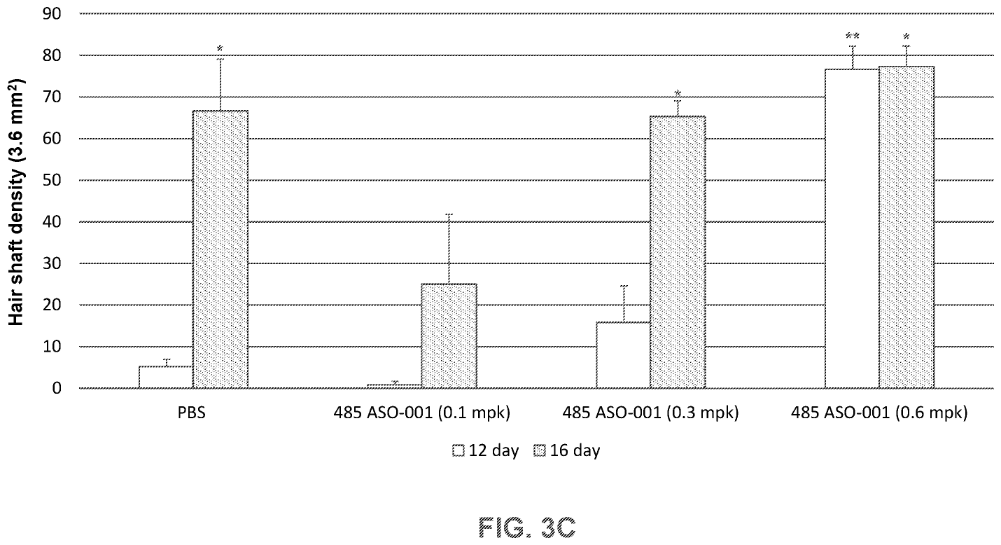

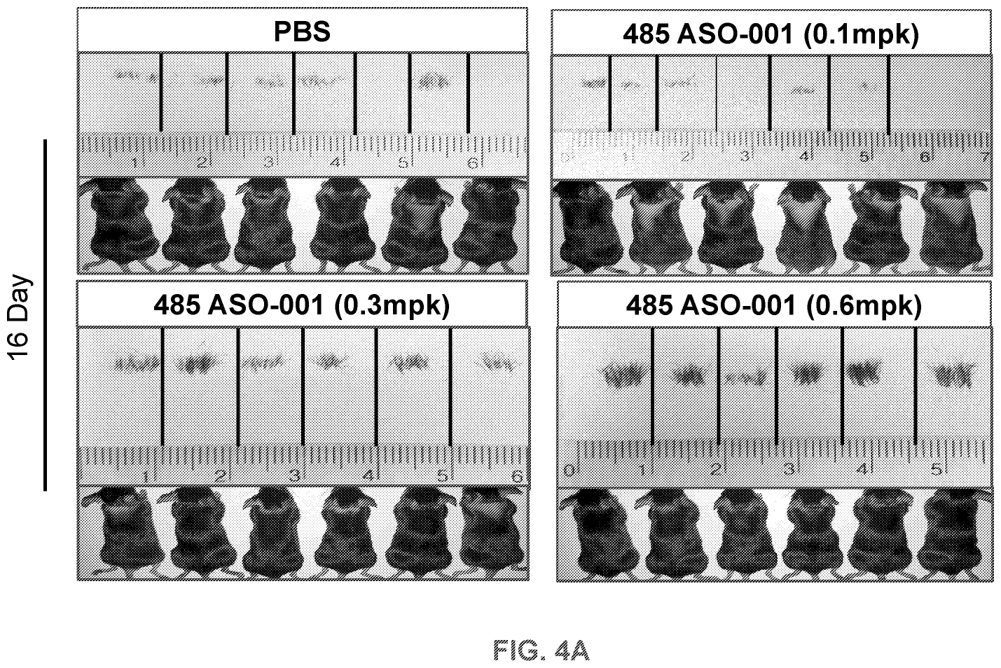

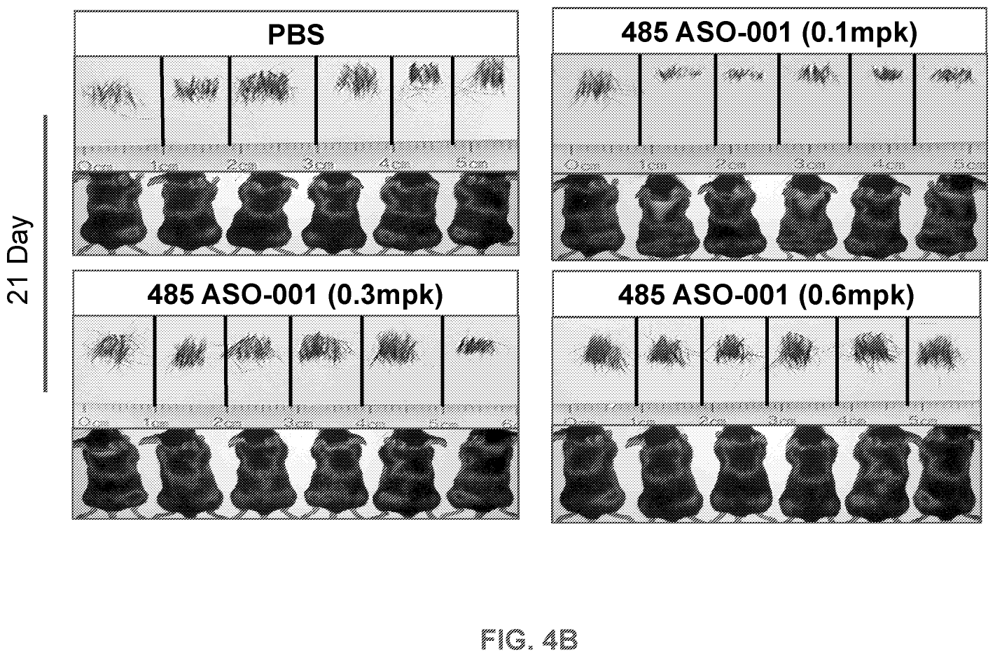

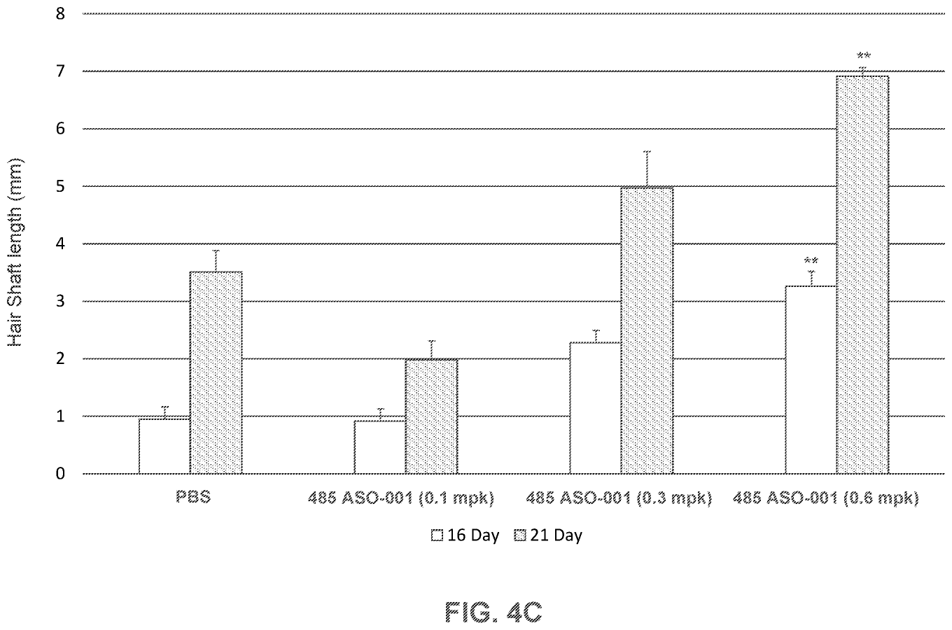

Furthermore, C57BL/6J Mice were divided into five groups (n=5) to evaluate the hair regrowth effect of the miR-485 inhibitor (485 ASO-001). Negative control (PBS), and positive control (2% Minoxidil), and 485 ASO-001 (0.3 mpk, and 0.6 mpk) were administered by twice (PD0 and PD7) intramuscular injection. The digital photographs of mice were taken post-depilation (pd.) on days 5 (FIG. 4D), 7 (FIG. 4E), 10 (FIG. 4F), 12 (FIG. 4G), 14 (FIG. 4H), and 16 (FIG. 4I) after treatment, and the hair growth area was analyzed via morphological observation by a score of 0-6 (FIG. 4J). There was a significant increase observed in hair growth score on days 7, 10, 12, 14, and 16 in 485 ASO-001 two times repeat intramuscular injection (0.3 mpk and 0.6 mpk) treated mice groups as compared to the PBS-treated control group and positive control Minoxidil treatment group (FIG. 4J). Furthermore, 485 ASO-001 (0.6 mpk-twice) treated mice group showed more hair regrowth compared to 485 ASO-001 (0.3 mpk-twice) treated mice group (FIG. 4J).

The above results demonstrate the dose-dependent efficacy of the miR-485 inhibitor (485 ASO-001) in inducing hair growth. For example, results showed that the miR-485 inhibitor (485 ASO-001) enhanced early indication of anagen hair growth in telegenic mice (see e.g., FIGS. 4D-4J). In particular, miR-485 inhibitor (485 ASO-001) (0.6 mpk-twice) treated mice group showed strongly activated and maintain anagen hair growth (FIGS. 4D-4J) as compared to PBS-treated control group and Minoxidil treatment group. These results suggest that miR-485 inhibitor (485 ASO-001), activated the transition from telogen to anagen phase, and therefore promoted hair growth.

### Example 4: Analysis of Dose Dependent Effect of miR-485 Inhibitor on Hair Density

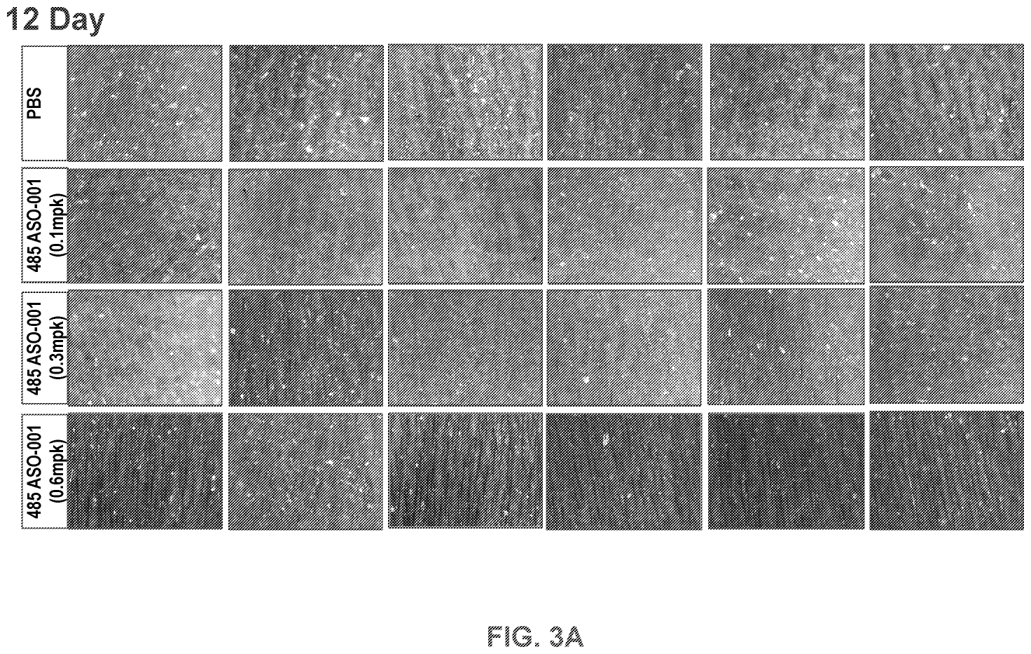

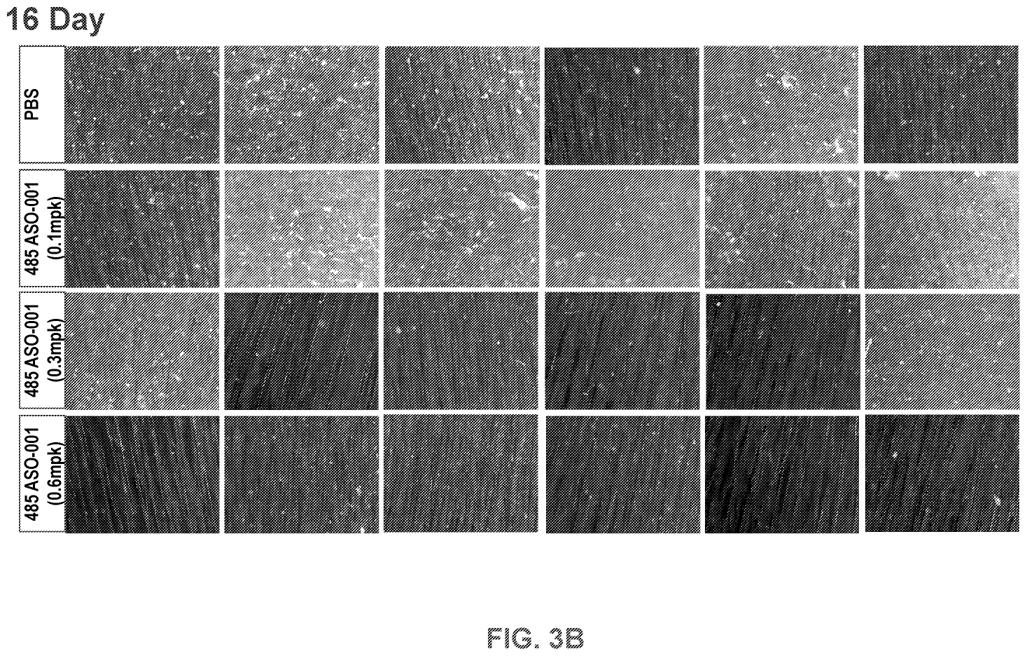

To further assess the hair regrowth effect of the miR-485 inhibitor (485 ASO-001) prepared as shown in Example 1 on depilated mice treated with PBS and 485 ASO-001, as described in Example 1, the hair density after treatment with 485 ASO-001 (0.1 mpk, 0.3 mpk, 0.6 mpk) was compared to the PBS-treated control group. Dermoscopic images of each mouse were acquired in the same region (3.6 mm²) of interscapular skin on days 12 (FIG. 3A) and 16 (FIG. 3B) using Kong, Bom-Viewer Plus software (Bomtech Electronics Co., Ltd., Seoul, Korea). Hair density was evaluated by analyzing the images (×200 magnification; actual area, 3.6 mm²).

As shown in FIG. 3C, there was a significant increase observed in the hair density on days 12 and 16 in 485 ASO-001 (0.6 mpk) treated mice groups compared to the PBS-treated control group. 485 ASO-001 (0.6 mpk) treated mice group showed increased hair density compared to 485 ASO-001 (0.1 mpk) and 485 ASO-001 (0.3 mpk) treated mice groups.

The above results demonstrate the dose-dependent efficacy of the miR-485 inhibitor (485 ASO-001) in increasing the hair density.

### Example 5: Analysis of Dose Dependent Effect of miR-485 Inhibitor on Hair Length

To further assess the hair regrowth effect of the miR-485 inhibitor (485 ASO-001) as prepared in Example 1 on depilated mice treated with PBS and 485 ASO-001, as described in Example 1, the hair length after treatment with 485 ASO-001 (0.1 mpk, 0.3 mpk, 0.6 mpk) was compared to the PBS-treated control group. After treatment hairs were plucked from representative areas in the depilated dorsal interscapular region of the back at days 16 (FIG. 4A) and 21 (FIG. 4B) and the average hair length from 30 hairs per mouse was calculated.

As shown in FIG. 4C, there was a significant increase observed in the hair length on day 16 in 485 ASO-001 (0.3 mpk and 0.6 mpk) treated mice groups compared to the PBS-treated control group.

The above results demonstrate the dose-dependent efficacy of the miR-485 inhibitor (485 ASO-001) in increasing the hair length.

### Example 6: Expression of miR-485-3p During Skin Development and Hair Follicle Cycling

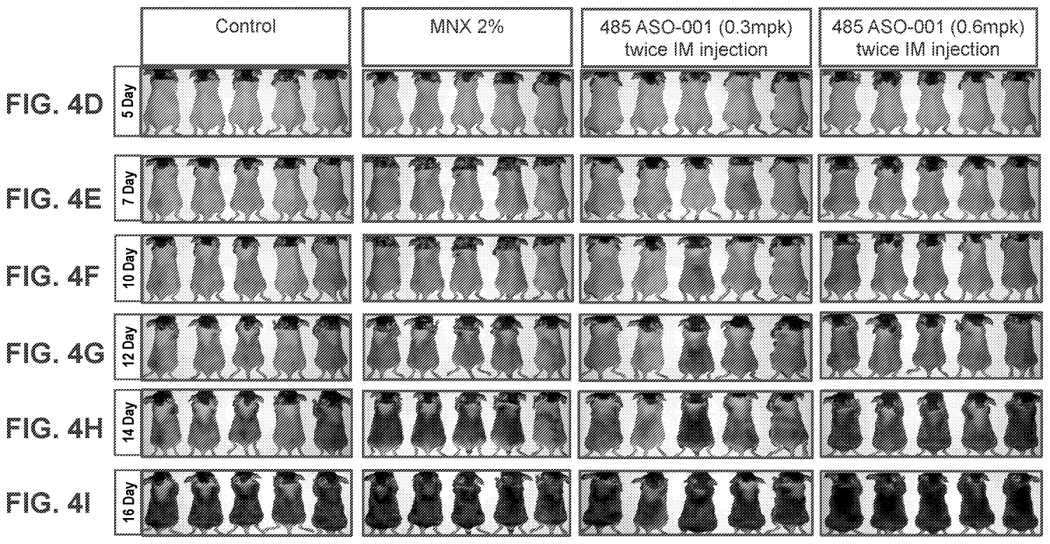

To test the effect of miR-485 inhibitor (485 ASO-001) on the miR-485-3p (FW7_mimic) expression level, tissue samples were obtained pdl 0 (FIG. 5A) and pd16 (FIG. 5B) of the murine hair cycle from an early anagen to the late anagen. miRNA expressions were decreased during the late anagen growth phase of the adult hair cycle, as compared with early anagen. There was a significant decreased of miRNA expressions observed miR-485 inhibitor (485 ASO-001) (0.6 mpk-twice) treated mice group on pd10 (p=0.01) and pd16 (p=0.03) as compared to (485 ASO-001) (0.3 mpk-twice) and PBS-treated control groups. The above results demonstrate the dose-dependent down regulation of expression of miR-485-3p by the miR-485 inhibitor (485 ASO-001).

### Example 7: Assessment of Dose Dependent Effect of miR-485 Inhibitor on Skin and Hair Follicle Density

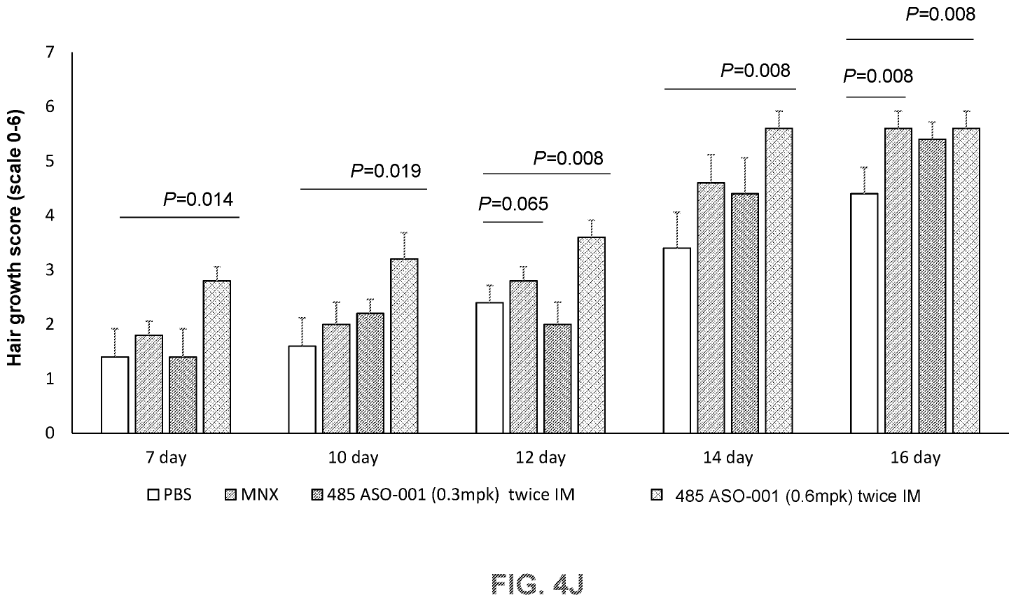

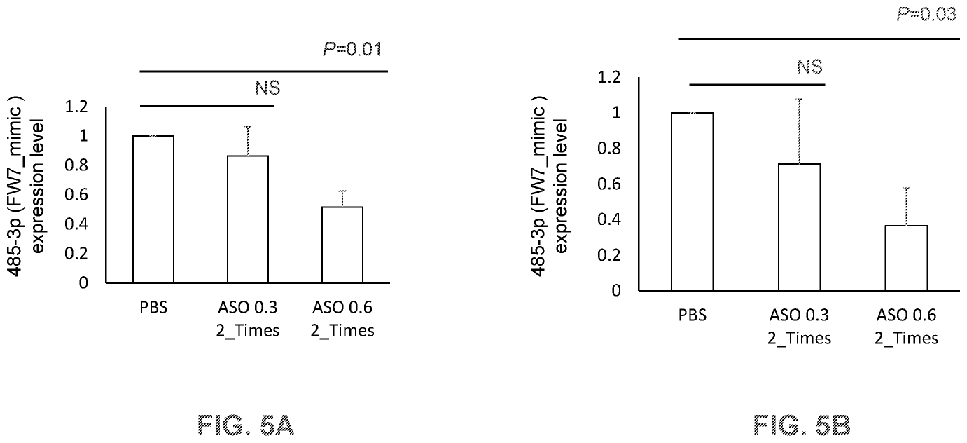

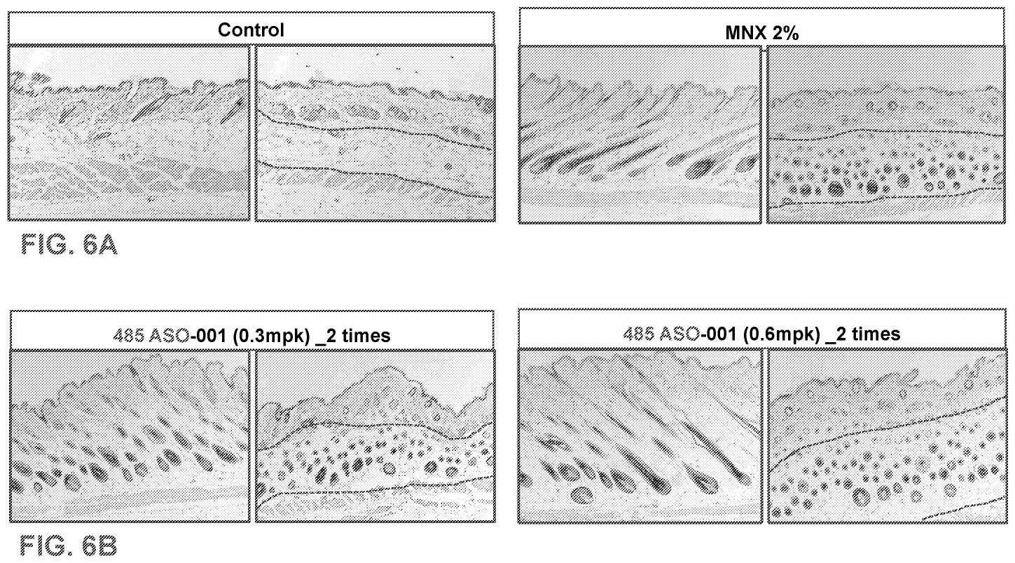

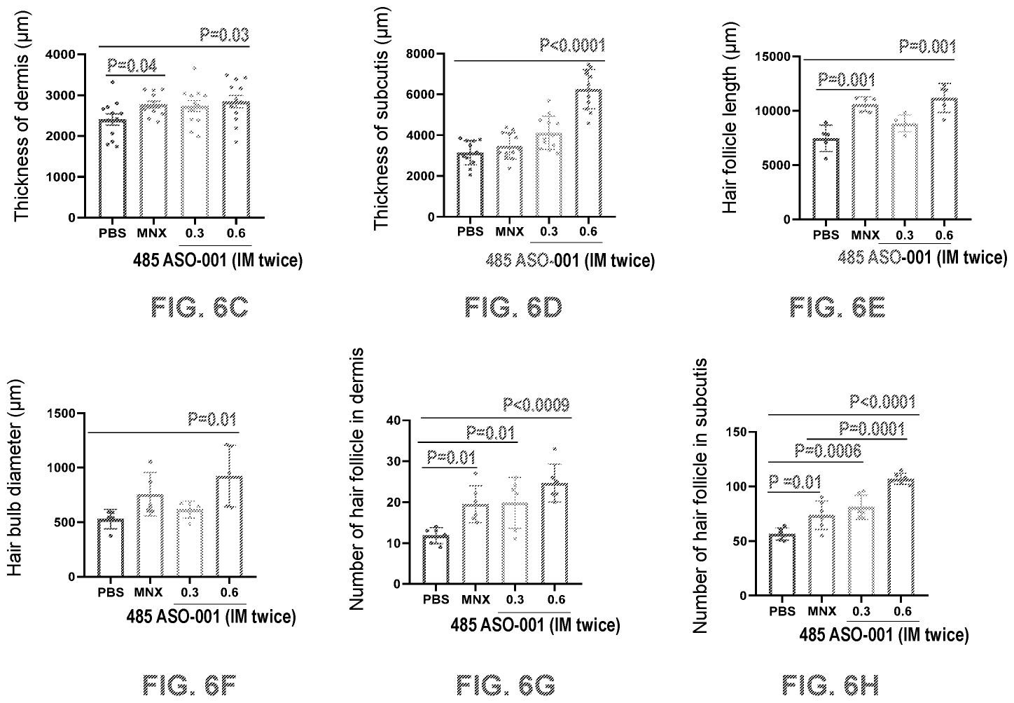

The effect of miR-485 inhibitor (485 ASO-001) (0.3 mpk and 0.6 mpk) on hair growth was further assessed by H&E staining (FIG. 6A, FIG. 6B, FIG. 7A, FIG. 7B). There was a significant increase observed in the dermis (p=0.03) (FIG. 6C), subcutis (p=0.0001) (FIG. 6D), hair follicle diameter (p=0.001) (FIG. 6E), hair bulb diameter (p=0.01) (FIG. 6F), hair follicle density in dermis (p<0.0009) (FIG. 6G), and hair follicle density in subcutis (p<0.0009) (FIG. 6H) on day 10 pd. There was a significant increase observed in the subcutis (p=0.0003) (FIG. 7C), hair follicle diameter (p=0.008) (FIG. 7F), hair follicle density in dermis (p<0.0001) (FIG. 7G), and hair follicle density in subcutis (p<0.0001) (FIG. 7H) in 485 ASO-001 (0.6 mpk-twice) treated mice groups compared to the PBS-treated control treated group. 485 ASO-001 (0.6 mpk) treated mice group showed increased number of hair follicle in subcutis (p<0.001) (FIG. 6H) on day 10 pd and on day 16 pd increased number of hair follicle in dermis (p=0.004) (FIG. 7G) compared to minoxidil-treated mice groups. Treatment with miR-485 inhibitor (485 ASO-001) (0.3 mpk and 0.6 mpk) (p<0.001) increased the hair follicle number in a dose-dependent manner compared to control (Figure FIG. 6G, FIG. 6H, FIG. 7G, FIG. 7H). Even though, minoxidil treatment also significantly (p<0.01) increased the hair follicle number as compared to the negative control, this increase was lower than that with miR-485 inhibitor (485 ASO-001) (0.6 mpk) treatment (Figure FIG. 6G, FIG. 6H). miR-485 inhibitor (485 ASO-001) (0.6 mpk) and minoxidil treatments significantly increased thickness of dermis (p<0.03 and p<0.04) as compared to the control group, but the minoxidil treatment increase in dermis and subcutis thickness was lower than that with miR-485 inhibitor (485 ASO-001) (0.6 mpk) (p<0.001) treatment (Figure FIG. 6C, FIG. 6D). miR-485 inhibitor (485 ASO-001) (0.6 mpk) treatment significantly increased diameter of hair bulb (p<0.01) compared to the control and minoxidil treatments group on day 10 pd. On day 16 pd, the treatment with miR-485 inhibitor (485 ASO-001) (0.3 mpk and 0.6 mpk) (p<0.001 and 0.0003) increased the thickness of subcutis in a dose-dependent manner compared to control.

### Example 8: miR-485 Inhibitor Increased the Wnt and β-Catenin Protein Expression

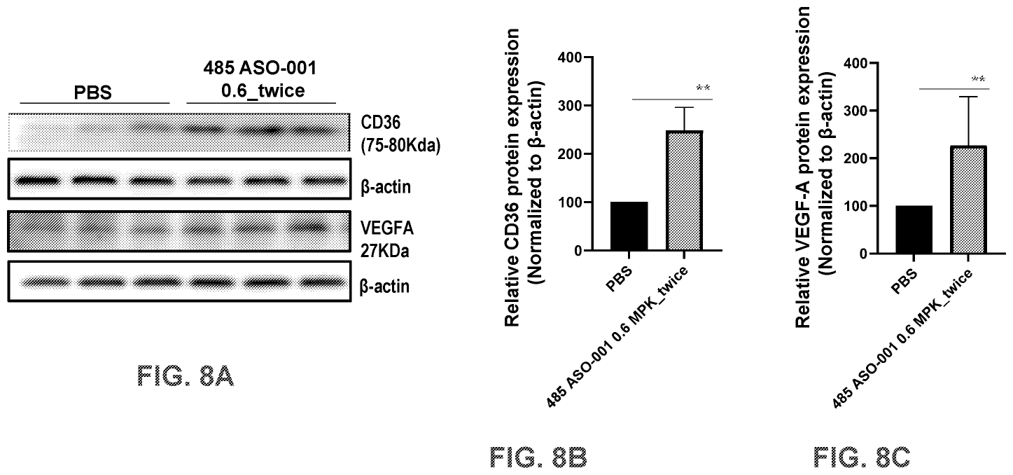

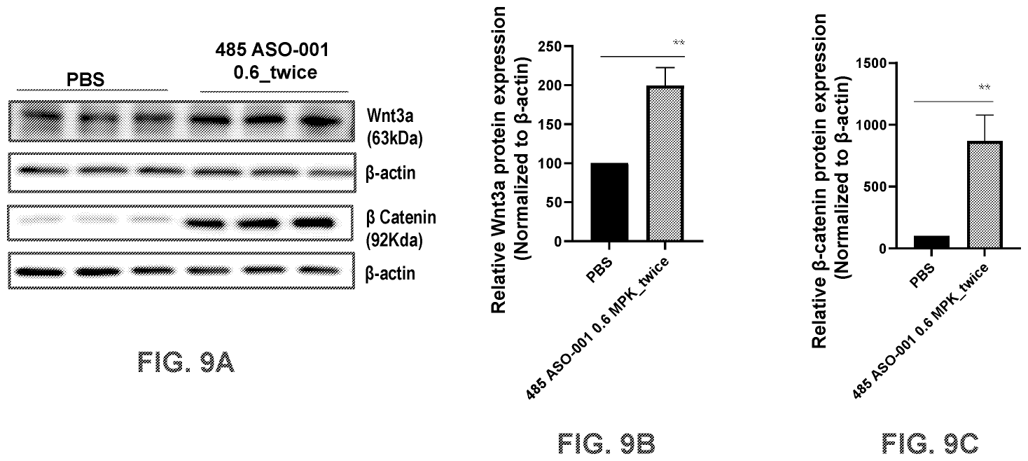

The effect of miR-485 inhibitor (485 ASO-001) on Wnt3a and β-catenin protein expression was assessed by Western blotting. The Wnt/β-catenin signaling is specifically involved in hair follicle morphogenesis, regeneration, and growth. Wnt3a induces hair growth due to the ability to activate the Wnt/β-catenin signaling pathway in dermal papilla (DP) cells. β-catenin is expressed in the dermal papilla and promotes anagen induction and duration, as well as keratinocyte regulation and differentiation. miR-485 inhibitor (485 ASO-001) (0.6 mpk) up regulated the expression of Wnt3a (FIG. 9B) and β-catenin (FIG. 9C). β-Catenin, which has been implicated in skin and hair follicle development, is an essential molecule in the Wnt signaling pathway. The above results demonstrate that miR-485 inhibitor (485 ASO-001) (0.6 mpk) significantly promotes the elongation of the hair shafts and the differentiation.

### Example 9: miR-485 Inhibitor Increased CD36 and VEGF-A Protein Expression

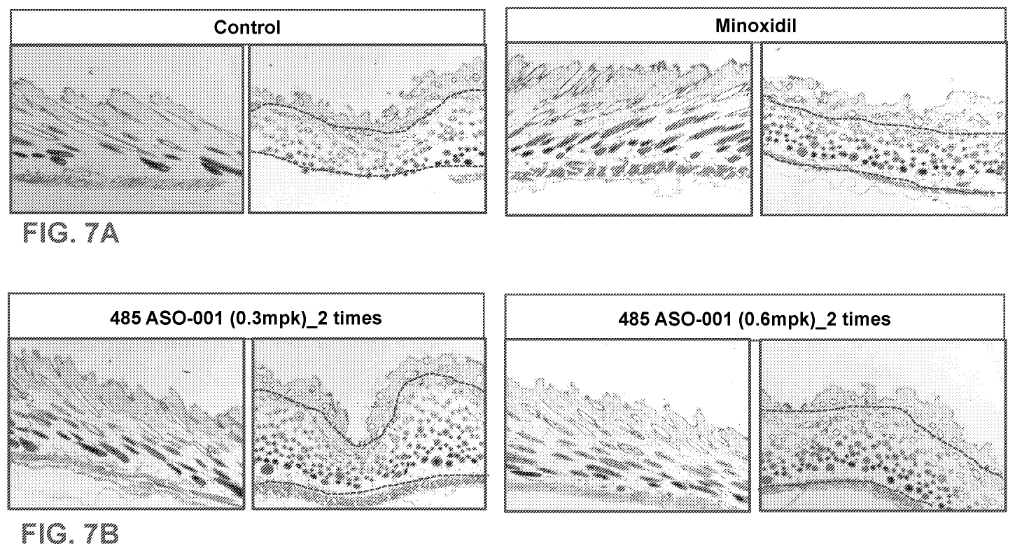

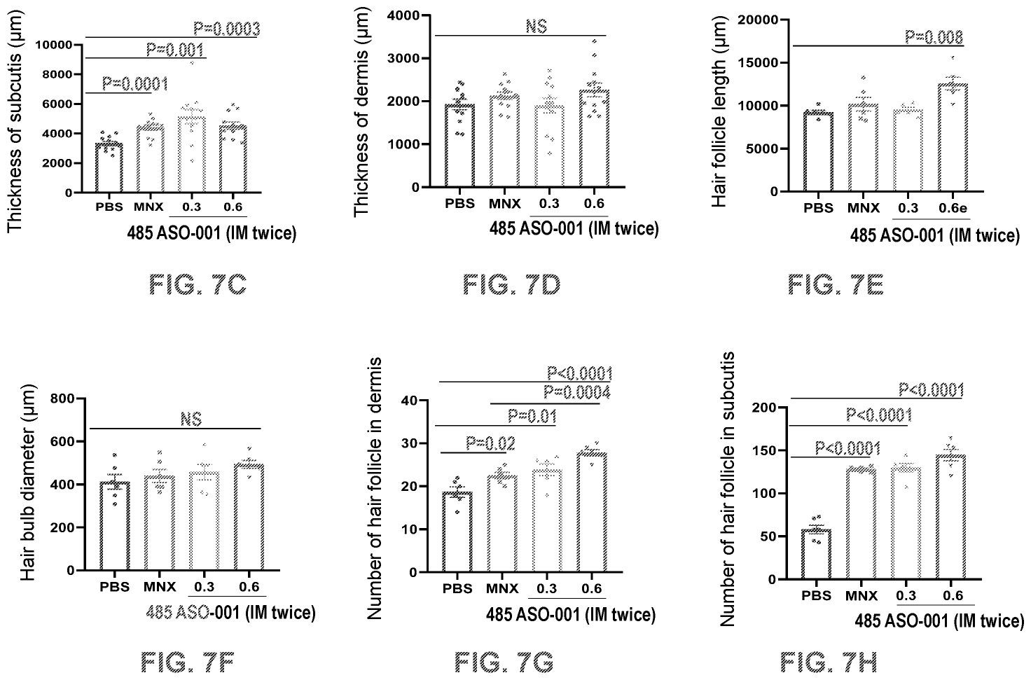

The effect of miR-485 inhibitor-485 ASO-001 on CD36 and vascular endothelial growth factor-A (VEGF-A) protein expression was assessed by Western blotting. Treatment with miR-485 inhibitor-485 ASO-001 (0.6 mpk) up regulated the protein expression level of CD36 (FIG. 8B) and VEGF-A (FIG. 8C) in compared to the PBS control group. VEGF-A is the most potent and specific vascular growth factor and a key regulator in physiological and pathological angiogenesis (blood capillary formation). VEGF-A levels are regulated through transcriptional control and mRNA stability.

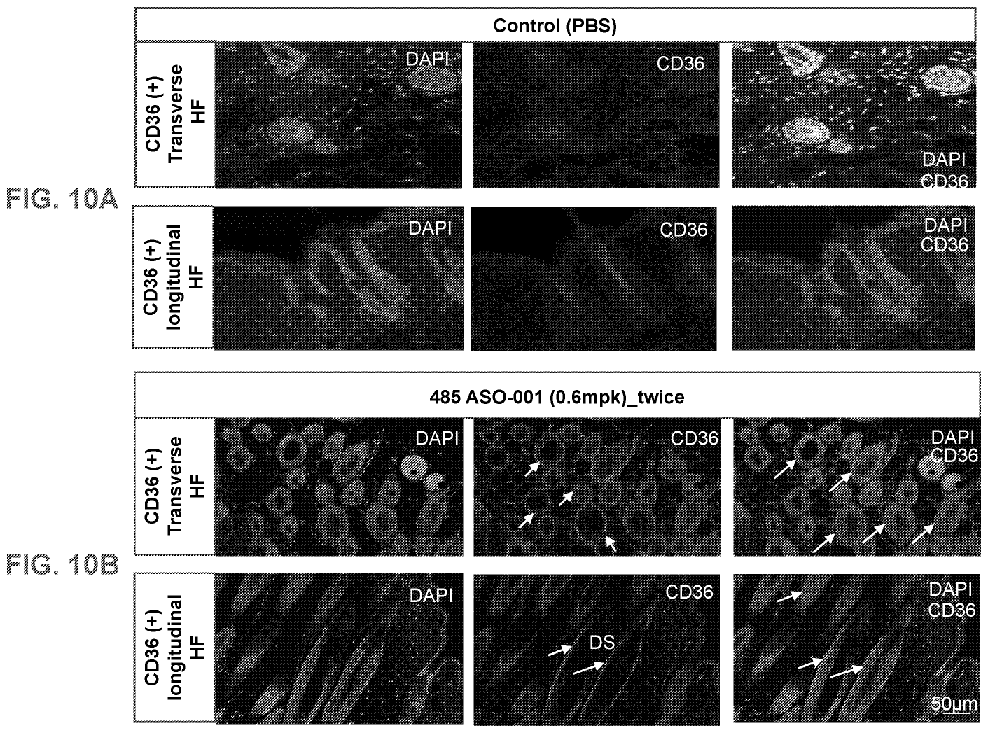

Mesenchyme of the murine pelage follicle is comprised of a follicle-lining smooth muscle known as the dermal sheath (DS). DS modulates blood capillaries in hair follicles in association with hair cycling. An immunofluorescence experiment was conducted to assess the effect of miR-485 inhibitor (485 ASO-001) (0.6 mpk) on the expression of CD36 in DS affected angiogenesis. Immunofluorescence results (FIG. 10B) showed that CD36 expressing cells increase in DS in miR-485 inhibitor (485 ASO-001) treated hair follicles. This can be associated with angiogenesis, particularly at anagen hair follicles.

It is to be appreciated that the Detailed Description section, and not the Summary and Abstract sections, is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

The contents of all cited references (including literature references, patents, patent applications, and websites) that can be cited throughout this application are hereby expressly incorporated by reference in their entirety for any purpose, as are the references cited therein.

What is claimed is:

1. A method of inducing hair growth in a subject in need thereof comprising administering to the subject a compound that inhibits miR-485 (“miRNA inhibitor”).

2. A method of increasing the hair density in a subject in need thereof comprising administering to the subject a compound that inhibits miR-485 (“miRNA inhibitor”).

3. A method of increasing the follicular density in a subject in need thereof comprising administering to the subject a compound that inhibits miR-485 (“miRNA inhibitor”).

4. A method of increasing the hair shaft thickness in a subject in need thereof comprising administering to the subject a compound that inhibits miR-485 (“miRNA inhibitor”).

5. A method of increasing the hair length in a subject in need thereof comprising administering to the subject a compound that inhibits miR-485 (“miRNA inhibitor”).

6. A method for preventing hair loss in a subject at risk of hair loss comprising administering to the subject a compound that inhibits miR-485 (“miRNA inhibitor”).

7. A method for reducing hair loss in a subject in need thereof comprising administering to the subject a compound that inhibits miR-485 (“miRNA inhibitor”).

8. A method of upregulating CD36 protein in a dermal sheath of hair follicle in a subject in need thereof comprising administering to the subject a compound that inhibits miR-485 (“miRNA inhibitor”).

9. The method of any one of claims 1 to 8, wherein the subject has one or more disorders selected from the group consisting of alopecia greata, androgenic alopecia, alopecia areata, alopecia universalis, involutional alopecia, trichotillomania, telogen effluvium, anagen effluvium, cicatricial, alopecia, scarring alopecia, scalp thinning, hair shaft abnormalities, infectious hair disorders, genetic disorders, and hair loss due to chemotherapy, hormonal imbalance, fungal infection, medication intake, chemical hair treatment, or aging.

10. The method any one of claims 1 to 8, wherein the subject is a human.

11. The method of any one of claims 1 to 10, wherein the miRNA inhibitor induces autophagy and/or treats or prevents inflammation.

12. The method of any one of claims 1 to 11, wherein the miRNA inhibitor induces phagocytosis.

13. The method of any one of claims 1 to 12, wherein the miRNA inhibitor inhibits miR485-3p.

14. The method of claim 13, wherein the miR485-3p comprises 5′-gucauacacggcucuccucucu-3′ (SEQ ID NO: 1).

15. The method of any one of claims 1 to 14, wherein the miRNA inhibitor comprises a nucleotide sequence comprising 5′- UGUAUGA-3′ (SEQ ID NO: 2) and wherein the miRNA inhibitor comprises about 6 to about 30 nucleotides in length.

16. The method of any one of claims 1 to 15, wherein the miRNA inhibitor comprises at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides at the 5′ of the nucleotide sequence.

17. The method of any one of claims 1 to 16, wherein the miRNA inhibitor comprises at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides at the 3′ of the nucleotide sequence.

18. The method of any one of claims 1 to 13 and 16 to 17, wherein the miRNA inhibitor has a sequence selected from the group consisting of: 5′-UGUAUGA-3′ (SEQ ID NO: 2), 5′-GUGUAUGA-3′ (SEQ ID NO: 3), 5′-CGUGUAUGA-3′ (SEQ ID NO: 4), 5′-CCGUGUAUGA-3′ (SEQ ID NO: 5), 5′-GCCGUGUAUGA-3′ (SEQ ID NO: 6), 5′-AGCCGUGUAUGA-3′ (SEQ ID NO: 7), 5′-GAGCCGUGUAUGA-3′ (SEQ ID NO: 8), 5′-AGAGCCGUGUAUGA-3′ (SEQ ID NO: 9), 5′-GAGAGCCGUGUAUGA-3′ (SEQ ID NO: 10), 5′-GGAGAGCCGUGUAUGA-3′ (SEQ ID NO: 11), 5′-AGGAGAGCCGUGUAUGA-3′ (SEQ ID NO: 12), 5′-GAGGAGAGCCGUGUAUGA-3′ (SEQ ID NO: 13), 5′-AGAGGAGAGCCGUGUAUGA-3′ (SEQ ID NO: 14), 5′-GAGAGGAGAGCCGUGUAUGA-3′ (SEQ ID NO: 15); 5′-UGUAUGAC-3′ (SEQ ID NO: 16), 5′-GUGUAUGAC-3′ (SEQ ID NO: 17), 5′-CGUGUAUGAC-3′ (SEQ ID NO: 18), 5′-CCGUGUAUGAC-3′ (SEQ ID NO: 19), 5′-GCCGUGUAUGAC-3′ (SEQ ID NO: 20), 5′-AGCCGUGUAUGAC-3′ (SEQ ID NO: 21), 5′-GAGCCGUGUAUGAC-3′ (SEQ ID NO: 22), 5′-AGAGCCGUGUAUGAC-3′ (SEQ ID NO: 23), 5′-GAGAGCCGUGUAUGAC-3′ (SEQ ID NO: 24), 5′-GGAGAGCCGUGUAUGAC-3′ (SEQ ID NO: 25), 5′-AGGAGAGCCGUGUAUGAC-3′ (SEQ ID NO: 26), 5′-GAGGAGAGCCGUGUAUGAC-3′ (SEQ ID NO: 27), 5′-AGAGGAGAGCCGUGUAUGAC-3′ (SEQ ID NO: 28), or 5′-GAGAGGAGAGCCGUGUAUGAC-3′ (SEQ ID NO: 29).

19. The method of any one of claims 1 to 13 and 16 to 17, wherein the miRNA inhibitor has a sequence selected from the group consisting of: 5′-TGTATGA-3′ (SEQ ID NO: 30), 5′-GTGTATGA-3′ (SEQ ID NO: 51), 5′-CGTGTATGA-3′ (SEQ ID NO: 52), 5′-CCGTGTATGA-3′ (SEQ ID NO: 53), 5′-GCCGTGTATGA-3′ (SEQ ID NO: 54), 5′-AGCCGTGTATGA-3′ (SEQ ID NO: 55), 5′-GAGCCGTGTATGA-3′ (SEQ ID NO: 35), 5′-AGAGCCGTGTATGA-3′ (SEQ ID NO: 56), 5′-GAGAGCCGTGTATGA-3′ (SEQ ID NO: 57), 5′-GGAGAGCCGTGTATGA-3′ (SEQ ID NO: 58), 5′-AGGAGAGCCGTGTATGA-3′ (SEQ ID NO: 59), 5′-GAGGAGAGCCGTGTATGA-3′ (SEQ ID NO: 60), 5′-AGAGGAGAGCCGTGTATGA-3′ (SEQ ID NO: 61), 5′-GAGAGGAGAGCCGTGTATGA-3′ (SEQ ID NO: 62); 5′-TGTATGAC-3′ (SEQ ID NO: 63), 5′-GTGTATGAC-3′ (SEQ ID NO: 64), 5′-CGTGTATGAC-3′ (SEQ ID NO: 65), 5′-CCGTGTATGAC-3′ (SEQ ID NO: 66), 5′-GCCGTGTATGAC-3′ (SEQ ID NO: 67), 5′-AGCCGTGTATGAC-3′ (SEQ ID NO: 68), 5′-GAGCCGTGTATGAC-3′ (SEQ ID NO: 69), 5′-AGAGCCGTGTATGAC-3′ (SEQ ID NO: 70), 5′-GAGAGCCGTGTATGAC-3′ (SEQ ID NO: 71), 5′-GGAGAGCCGTGTATGAC-3′ (SEQ ID NO: 72), 5′-AGGAGAGCCGTGTATGAC-3′ (SEQ ID NO: 73), 5′-GAGGAGAGCCGTGTATGAC-3′ (SEQ ID NO: 74), 5′-AGAGGAGAGCCGTGTATGAC-3′ (SEQ ID NO: 75), and 5′-GAGAGGAGAGCCGTGTATGAC-3′ (SEQ ID NO: 76).

20. The method of any one of claims 1 to 18, wherein the sequence of the miRNA inhibitor is at least about 50%, at least about 55%, at least about 60%, at least about 65%, at least about 70%, at least about 75%, at least about 80%, at least about 85%, at least about 90%, or at least about 95% sequence identity to 5′-AGAGGAGAGCCGUGUAUGAC -3′ (SEQ ID NO: 28) or 5′-AGAGGAGAGCCGTGTATGAC-3′ (SEQ ID NO: 77).

21. The method of claim 19, wherein the miRNA inhibitor has a sequence that has at least 90% similarity to 5′-AGAGGAGAGCCGUGUAUGAC-3′ (SEQ ID NO: 28) or 5′-AGAGGAGAGCCGTGTATGAC-3′ (SEQ ID NO: 77).

22. The method of any one of claims 1 to 20, wherein the miRNA inhibitor comprises the nucleotide sequence 5′- AGAGGAGAGCCGUGUAUGAC-3′ (SEQ ID NO: 28) or 5′-AGAGGAGAGCCGTGTATGAC-3′ (SEQ ID NO: 77) with one substitution or two substitutions.

23. The method of any one of claims 1 to 21, wherein the miRNA inhibitor comprises the nucleotide sequence 5′-AGAGGAGAGCCGUGUAUGAC -3′ (SEQ ID NO: 28) or 5′-AGAGGAGAGCCGTGTATGAC-3′ (SEQ ID NO: 77).

24. The method of claim 23, wherein the miRNA inhibitor comprises the nucleotide sequence 5′-AGAGGAGAGCCGUGUAUGAC-3′ (SEQ ID NO: 28).

25. The method of any one of claims 1 to 22, wherein the miRNA inhibitor comprises at least one modified nucleotide.

26. The method of claim 25, wherein the at least one modified nucleotide is a locked nucleic acid (LNA), an unlocked nucleic acid (UNA), an arabino nucleic acid (ABA), a bridged nucleic acid (BNA), and/or a peptide nucleic acid (PNA).

27. The method of any one of claims 1 to 26, wherein the miRNA inhibitor comprises a backbone modification.

28. The method of claim 27, wherein the backbone modification is a phosphorodiamidate morpholino oligomer (PMO) and/or phosphorothioate (PS) modification.

29. The method of any one of claims 1 to 28, wherein the miRNA inhibitor is delivered in a delivery agent.

30. The method of claim 29, wherein the delivery agent is a micelle, an exosome, a lipid nanoparticle, an extracellular vesicle, or a synthetic vesicle.

31. The method of any one of claims 1 to 30, wherein the miRNA inhibitor is delivered by a viral vector.

32. The method of claim 31, wherein the viral vector is an AAV, an adenovirus, a retrovirus, or a lentivirus.

33. The method of claim 32, wherein the viral vector is an AAV that has a serotype of AAV2, AAV3, AAV4, AAV5, AAV6, AAV7, AAV8, AAV9, AAV10, or any combination thereof.

34. The method of any one claims 1 to 33, wherein the miRNA inhibitor is delivered with a delivery agent.

35. The method of claim 34, wherein the delivery agent comprises a micelle, an exosome, a lipidoid, a liposome, a lipoplex, a lipid nanoparticle, an extracellular vesicle, a synthetic vesicle, a polymeric compound, a peptide, a protein, a cell, a nanoparticle mimic, a nanotube, a conjugate, a viral vector, or combinations thereof.

36. The method of claim 34 or 35, wherein the delivery agent comprises a cationic carrier unit comprising:
[WP]-L1-[CC]-L2-[AM] (formula I)
or
[WP]-L1-[AM]-L2-[CC] (formula II)
wherein
WP is a water-soluble biopolymer moiety;
CC is a positively charged carrier moiety;
AM is an adjuvant moiety; and,
L1 and L2 are independently optional linkers, and
wherein when mixed with a nucleic acid at an ionic ratio of about 1:1, the cationic carrier unit forms a micelle.

37. The method of claim 36, wherein the miRNA inhibitor interacts with the cationic carrier unit via an ionic bond.

38. The method of claim 36 or 37, wherein the water-soluble polymer comprises poly(alkylene glycols), poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyglycerol, polyphosphazene, polyoxazolines (“POZ”) poly(N-acryloylmorpholine), or any combinations thereof.

39. The method of any one of claims 36 to 38, wherein the water-soluble polymer comprises polyethylene glycol (“PEG”), polyglycerol, or poly(propylene glycol) (“PPG”).

40. The method of any one of claims 36 to 39, wherein the water-soluble polymer comprises: 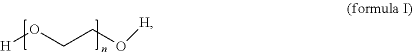
wherein n is 1-1000.

41. The method of claim 40, wherein the n is at least about 110, at least about 111, at least about 112, at least about 113, at least about 114, at least about 115, at least about 116, at least about 117, at least about 118, at least about 119, at least about 120, at least about 121, at least about 122, at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, or at least about 141.

42. The method of claim 40, wherein the n is about 80 to about 90, about 90 to about 100, about 100 to about 110, about 110 to about 120, about 120 to about 130, about 140 to about 150, about 150 to about 160.

43. The method of any one of claims 36 to 42, wherein the water-soluble polymer is linear, branched, or dendritic.

44. The method of any one of claims 36 to 43, wherein the cationic carrier moiety comprises one or more basic amino acids.

45. The method of claim 44, wherein the cationic carrier moiety comprises at least three, at least four, at least five, at least six, at least seven, at least eight, at least nine, at least ten, at least 11, at least 12, at least 13, at least 14, at last 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 basic amino acids.

46. The method of claim 45, wherein the cationic carrier moiety comprises about 30 to about 50 basic amino acids.

47. The method of claim 45 or 46, wherein the basic amino acid comprises arginine, lysine, histidine, or any combination thereof.

48. The method of any one of claims 36 to 47, wherein the cationic carrier moiety comprises about 40 lysine monomers.

49. The method of any one of claims 36 to 48, wherein the adjuvant moiety is capable of modulating an immune response, an inflammatory response, and/or a tissue microenvironment.

50. The method of any one of claims 36 to 49 wherein the adjuvant moiety comprises an imidazole derivative, an amino acid, a vitamin, or any combination thereof.

51. The method of claim 50, wherein the adjuvant moiety comprises: 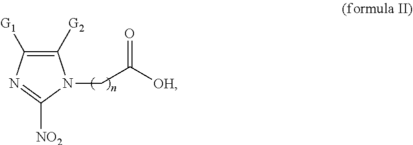
wherein each of G1 and G2 is H, an aromatic ring, or 1-10 alkyl, or G1 and G2 together form an aromatic ring, and wherein n is 1-10.

52. The method of claim 50, wherein the adjuvant moiety comprises nitroimidazole.

53. The method of claim 50, wherein the adjuvant moiety comprises metronidazole, tinidazole, nimorazole, dimetridazole, pretomanid, ornidazole, megazol, azanidazole, benznidazole, or any combination thereof.

54. The method of any one of claims 36 to 50, wherein the adjuvant moiety comprises an amino acid.

55. The method of claim 54, wherein the adjuvant moiety comprises 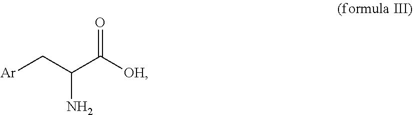
wherein Ar is 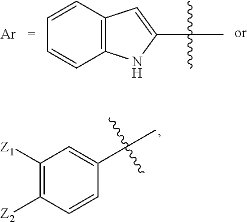
and wherein each of Z1 and Z2 is H or OH.

56. The method of any one of claims 36 to 50, wherein the adjuvant moiety comprises a vitamin.

57. The method of claim 56, wherein the vitamin comprises a cyclic ring or cyclic hetero atom ring and a carboxyl group or hydroxyl group.

58. The method of claim 57, wherein the vitamin comprises: 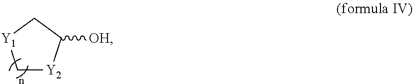
wherein each of Y1 and Y2 is C, N, O, or S, and wherein n is 1 or 2.

59. The method of any one of claims 56 to 58, wherein the vitamin is selected from the group consisting of vitamin A, vitamin B1, vitamin B2, vitamin B3, vitamin B6, vitamin B7, vitamin B9, vitamin B12, vitamin C, vitamin D2, vitamin D3, vitamin E, vitamin M, vitamin H, and any combination thereof.

60. The method of any one of claims 56 to 59, wherein the vitamin is vitamin B3.

61. The method of any one of claims 56 to 60, wherein the adjuvant moiety comprises at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, or at least about 20 vitamin B3.

62. The method of claim 61, wherein the adjuvant moiety comprises about 10 vitamin B3.

63. The method of any one of claims 56 to 62, wherein the delivery agent comprises about a water-soluble biopolymer moiety with about 120 to about 130 PEG units, a cationic carrier moiety comprising a poly-lysine with about 30 to about 40 lysines, and an adjuvant moiety with about 5 to about 10 vitamin B3.

64. The method of any one of claims 56 to 63, wherein the delivery agent is associated with the miRNA inhibitor, thereby forming a micelle.

65. The method of claim 64, wherein the association is a covalent bond, a non-covalent bond, or an ionic bond.

66. The method of claim 64 or 65, wherein the cationic carrier unit and the miRNA inhibitor in the micelle is mixed in a solution so that the ionic ratio of the positive charges of the cationic carrier unit and the negative charges of the miRNA inhibitor is about 1:1.

67. The method of any one of claims 64 to 66, wherein the cationic carrier unit is capable of protecting the miRNA inhibitor from enzymatic degradation.

68. The method of any one of claims 1 to 67, wherein the miRNA inhibitor is administered parenthetically, intramuscularly, subcutaneously, ophthalmic, intravenously, intraperitoneally, intradermally, intraorbitally, intracerebrally, intracranially, intracerebroventricularly, intraspinally, intraventricular, intrathecally, intracistemally, intracapsularly, intratumorally, topically, or any combination thereof.

69. The method of any one of claims 1 to 68, wherein the miRNA inhibitor is administered to a skin area where promoting hair growth is needed by spread, spray, steam, or injection.

70. The method of any one of claims 1 to 68, wherein the miRNA inhibitor is administered topically to a skin area where promoting hair growth is needed.

71. The method any one of claims 1 to 68, wherein the miRNA inhibitor is formulated in a form selected from the group consisting of an ointment, a shampoo, a conditioner, a lotion, a tonic, a gel, and a mousse.

72. The method of any one of claims 1 to 68, wherein the administering step is performed by soaking or bathing the subject in the miRNA inhibitor formulated in a form selected from the group consisting of an ointment, a shampoo, a conditioner, a lotion, a tonic, a gel, and a mousse.
